(19) United States
(12) Patent Application Publication
(10) Pub. No.: US 2018/0154170 A1
(43) Pub. Date: Jun. 7, 2018

(54) METHODS AND COMPOSITIONS FOR TREATING MENTAL DISORDERS AND CONDITIONS

(71) Applicant: Massachusetts Institute of Technology, Cambridge, MA (US)

(72) Inventors: Susumu Tonegawa, Newton, MA (US); Steve Ramirez Moreno, Cambridge, MA (US); Joshua Kim, Boston, MA (US); Xu Liu, Somerville, MA (US)

(73) Assignee: Massachusetts Institute of Technology, Cambridge, MA (US)

(21) Appl. No.: 15/737,156

(22) PCT Filed: Jun. 16, 2016
(86) PCT No.: PCT/US16/37877

§ 371 Date: Dec. 15, 2017

Related U.S. Application Data
(60) Provisional application No. 62/180,350, filed on Jun. 16, 2015.

(51) Int. Cl. A61N 5/06

(52) U.S. Cl. CPC A61N 5/0601; A61N 5/0622; A61N 2005/063; A61N 2005/0612; A61B 2503/40

(57) ABSTRACT

The invention, in some aspects, relates to methods to alter activity in cells and the use of such method to treat disorders and conditions. The methods involve, in part, expressing stimulus-activated opsin polypeptides in neurons involved in memory and behavior and activating the opsin polypeptides to modulate activity of cells in which they are expressed and/or cells to which the cells that express the opsin polypeptides project.

### RELATED APPLICATIONS

This application claims benefit under 35 U.S.C. § 119(e) of U.S. Provisional application Ser. No. 62/180,350 filed Jun. 16, 2015, the disclosure of which is incorporated by reference herein in its entirety.

### FIELD OF THE INVENTION

The invention, in some aspects, relates to methods and compositions for treating mental disorders and conditions.

### BACKGROUND OF THE INVENTION

Stress is considered a potent environmental risk factor for many behavioral abnormalities, including anxiety and mood disorders (1, 2). Animal models can exhibit limited but quantifiable behavioral impairments resulting from chronic stress, including deficits in motivation, abnormal responses to behavioral challenges, and anhedonia (3-5). The hippocampus is thought to negatively regulate the stress response and to mediate various cognitive and mnemonic aspects of stress-induced impairments (2,3,5), although the neuronal underpinnings sufficient to support behavioral improvements are largely unknown.

Another brain region that is believed to be involved in behavior and emotions is the basolateral complex of the amygdala, which consists of two intimately juxtaposed nucleithe lateral nucleus (LA) and basolateral nucleus (BLA)(38,39). The BLA is a cortical-like brain structure consisting of non-laminarly organized excitatory pyramidal neurons intermingled with populations of genetically defined interneurons (40-43). The BLA is activated by negative and positive emotional stimuli, and is necessary and sufficient for emotional behaviors and associations (44-51). Despite the important role of the BLA in the expression and regulation of emotional behaviors, it has not been determined whether the BLA pyramidal neurons that contribute to negative and positive behaviors (negative neurons and positive neurons) are structurally distinct, let alone, genetically distinguishable (52). Furthermore, a neural circuit sub-serving the antagonistic nature of emotional behaviors has yet to be identified.

### SUMMARY OF THE INVENTION

According to an aspect of the invention, methods of aiding in a treatment of a mental disease or a condition in a subject, the methods including: expressing in a first cell in a subject in need of such treatment, a stimulus-activated opsin polypeptide in an amount effective to treat a mental disease or condition in the subject; wherein activating the first cell reactivates a positive memory in the subject; contacting the expressed stimulus-activated opsin polypeptide with a stimulus suitable to activate the stimulus-activated opsin polypeptide; and modulating the contact of the stimulus with the stimulus-activated opsin polypeptide to reactivate the positive memory engram in the subject, wherein the reactivation of the positive memory aids in the treatment of the mental disease or the condition in the subject. In certain embodiments, the first cell is a hippocampal neuron of the subject, optionally is a dorsal hippocampal neuron. In some embodiments, first cell is in the dentate gyrus of the hippocampus. In some embodiments, the first cell projects to at least one second cell in the basal lateral amygdala (BLA) of the subject. In certain embodiments, the second cell is a parvocellular pyramidal neuron in the BLA of the subject. In certain embodiments, the second cell is a Ppplrlb⁺-expressing cell. In some embodiments, the suitable stimulus comprises illumination. In certain embodiments, a characteristic of the illumination includes one or more of: a wavelength of the illumination, a time period of the illumination, a frequency of two or more periods of illumination, an interval between two or more illumination periods, an intensity of the illumination. In some embodiments, re-activation of positive memory comprises reactivation of a positive memory engram. In some embodiments, the stimulation is chronic stimulation. In some embodiments, the stimulation is acute stimulation. In certain embodiments, the mental disorder or condition is depression or post-traumatic stress disorder (PTSD). In some embodiments, the stimulus-activated opsin polypeptide comprises a light-activated opsin polypeptide. In some embodiments, wherein the method also includes: altering one or more additional treatments administered to the subject to treat or assist in treating the mental disease or condition. In certain embodiments, altering an additional treatment includes: starting or increasing administration of a therapeutic agent to the subject, reducing or stopping administration of a therapeutic agent to the subject; starting or increasing administration of a behavioral therapy to the subject, reducing or stopping administration of a behavioral therapy to the subject, starting or increasing administration of a deep brain stimulation therapy to the subject, reducing or stopping administration of a deep brain stimulation therapy to the subject, administering a surgical therapy to the subject, starting or increasing administering a cognitive therapy to the subject, reducing or stopping administering a cognitive therapy to the subject, starting or increasing administering a counseling therapy to the subject, or reducing or stopping administering a counseling therapy to the subject. In some embodiments, the method also includes: exposing the subject to a positive experience sufficient to activate one or more of the first cells in the hippocampus of the subject. In some embodiments, exposing the subject occurs at one or more of: prior to (a) expressing in a first cell in a subject in need of such treatment the stimulus-activated opsin polypeptide and (b) contacting the expressed stimulus-activated opsin with a stimulus suitable to activate the opsin polypeptide. In some embodiments, re-activating the positive memory induces neurogenesis in the dentate gyrus of the subject. In certain embodiments, the first cell is in the basal lateral amygdala of the subject. In some embodiments, the first cell is a parvocellular pyramidal neuron in the BLA of the subject. In some embodiments, the first cell is a Ppplrlb⁺-expressing cell. In certain embodiments, the method also includes inhibiting a Rspo2⁺-expressing cell in the subject. In some embodiments, the inhibiting is at a time that is one or more of: prior to, concurrent with, or subsequent to the reactivation of the positive memory in the subject.

According to another aspect of the invention, methods of conditioning a positive behavior in a subject to a neutral environmental context are provided, the methods comprising: expressing in one or more cells in a subject a stimulus-activated opsin polypeptide, wherein the cell in which the stimulus-activated opsin polypeptide expressed is a Ppplrlb⁺-expressing cell or is a cell that when activated, activates a Ppplrlb⁺-expressing cell in the subject; activating the expressed stimulus-activated opsin polypeptide; and exposing the subject to a neutral environmental context at a time simultaneous with the activation of the expressed stimulus-activated opsin polypeptide; wherein the simultaneous activation and exposure conditions a positive behavior in the subject to the neutral environmental context. In some embodiments, the Ppplrlb⁺-expressing cell is a basal lateral amygdala (BLA) cell. In some embodiments, the method also includes activating the one or more Ppplrlb⁺-expressing cells in the positively conditioned subject. In certain embodiments, activating the one or more Ppplrlb⁺-expressing cells comprises activating a hippocampal cell that projects to the one or more Ppplrlb⁺-expressing cells. In some embodiments, the hippocampal cell is a dentate gyrus cell. In some embodiments, activating the one or more Ppplrlb⁺-expressing cells in the positively conditioned subject, assists in the treatment of a mental disease or condition in the subject. In certain embodiments, the stimulus-activated opsin polypeptide is an excitatory opsin polypeptide. In some embodiments, the stimulus-activated opsin polypeptide is a light-activated opsin polypeptide.

According to another aspect of the invention, methods of conditioning a negative behavior in a subject to a neutral environmental context are provided, the methods comprising: expressing in one or more cells in a subject a stimulus-activated opsin polypeptide, wherein the stimulus-activated opsin polypeptide is expressed in an Rspo2⁺-expressing cell or is expressed in a cell that when activated, activates an Rspo2⁺-expressing cell in the subject: activating the expressed stimulus-activated opsin polypeptide; and exposing the subject to a neutral environmental context at a time simultaneous with the activation of the expressed stimulus-activated opsin polypeptide; wherein the simultaneous activation and exposure conditions a negative behavior in the subject to the neutral environmental context. In some embodiments, the Rspo2⁺-expressing cell is a basal lateral amygdala (BLA) cell. In some embodiments, the method also includes activating one or more Rspo2⁺-expressing cells in the negatively conditioned subject. In certain embodiments, activating the one or more Rspo2⁺-expressing cells comprises activating a hippocampal cell that projects to the one or more Rspo2⁺-expressing cells. In some embodiments, the hippocampal cell is a dentate gyrus cell. In some embodiments, activating the one or more Rspo2⁺-expressing cells in the negatively conditioned subject, assists in the treatment of a mental disorder or condition in the subject. In some embodiments, the stimulus-activated opsin polypeptide is an inhibitory opsin polypeptide. In certain embodiments, the stimulus-activated opsin polypeptide is an excitatory opsin polypeptide. In some embodiments, the stimulus-activated opsin polypeptide is a light-activated opsin polypeptide.

According to another aspect of the invention, pharmaceutical compositions for inhibiting Rspo2⁺-expressing cell activity in a subject are provided, the pharmaceutical compositions comprising: a stimulus-activated opsin compound in an amount effective to inhibit activity of an Rspo2⁺-expressing cell in a subject, wherein expression of the stimulus-activated ion opsin in a cell in the subject and exposure of the expressed stimulus-activated opsin to a suitable stimulus, inhibits the Rspo2⁺-expressing cell activity in the subject. In some embodiments, the Rspo2⁺-expressing cell is in the basal lateral amygdala (BLA) of the subject. In some embodiments, the Rspo2⁺-expressing cell is a magnocellular pyramidal cell in the BLA. In some embodiments, the stimulus-activated opsin polypeptide is a light-activated opsin polypeptide. In certain embodiments, the stimulus-activated opsin is expressed in a cell in the subject that is upstream from the Rspo2⁺-expressing cell. In some embodiments, the upstream cell is a hippocampal cell. In some embodiments, the hippocampal cell is a dentate gyrus cell. In certain embodiments, the pharmaceutical composition also includes a pharmaceutically acceptable carrier. In some embodiments, the pharmaceutical composition also includes one or more of a: trafficking agent, targeting agent, and detectable label.

According to another aspect of the invention, pharmaceutical compositions for exciting/activating Rspo2⁺-expressing cell activity in a subject are provided, the pharmaceutical compositions include a stimulus-activated opsin compound in an amount effective to excite/activate activity of an Rspo2⁺-expressing cell in a subject, wherein expression of the stimulus-activated ion opsin in a cell in the subject and exposure of the expressed stimulus-activated opsin to a suitable stimulus, activates the Rspo2⁺-expressing cell activity in the subject.

According to another aspect of the invention, pharmaceutical compositions for activating a Ppplrlb⁺-expressing cell in a subject are provided. The pharmaceutical compositions comprise: a stimulus-activated opsin compound in an amount effective to stimulate activity of a Ppplrlb⁺-expressing cell in the subject, wherein expression of the stimulus-activated opsin in a cell in the subject and contact of the expressed stimulus-activated opsin with a suitable light, stimulates the Ppplrlb⁺-expressing cell in a subject. In some embodiments, the Ppplrlb⁺-expressing cell is in the basal lateral amygdala (BLA) of the subject. In some embodiments, the Ppplrlb⁺-expressing cell is a parvocellular pyramidal cell in the BLA. In some embodiments, the stimulus-activated opsin polypeptide is a light-activated opsin polypeptide. In certain embodiments, the stimulus-activated opsin is expressed in a cell in the subject that is upstream from the Ppplrlb⁺-expressing cell. In some embodiments, the upstream cell is a hippocampal cell. In some embodiments, the hippocampal cell is a dentate gyrus cell. In some embodiments, the pharmaceutical composition also includes a pharmaceutically acceptable carrier. In some embodiments, the pharmaceutical composition also includes one or more of a: trafficking agent, targeting agent, and detectable label.

According to another aspect of the invention, pharmaceutical compositions for inhibiting a Ppplrlb⁺-expressing cell in a subject are provided. The pharmaceutical compositions comprise: a stimulus-activated opsin compound in an amount effective to inhibit activity of a Ppplrlb⁺-expressing cell in the subject, wherein expression of the stimulus-activated opsin in a cell in the subject and contact of the expressed stimulus-activated opsin with a suitable light, inhibits the Ppplrlb⁺-expressing cell in a subject.

According to another aspect of the invention, methods of activating a Ppplrlb⁺-expressing cell in a subject are provided. The methods comprise: expressing in one or more cells in a subject a stimulus-activated opsin polypeptide, wherein the cell in which the stimulus-activated opsin polypeptide is expressed in a Ppplrlb⁺-expressing cell or in a cell that when activated, activates a Ppplrlb⁺-expressing cell in the subject: and activating the expressed stimulus-activated opsin polypeptide; wherein the activation stimulus-activated opsin polypeptide activates the Ppplrlb⁺-expressing cell in the subject. In certain embodiments, the Ppplrlb⁺-expressing cell is a basal lateral amygdala (BLA) cell. In some embodiments, the Ppplrlb⁺-expressing is a parvocellular pyramidal cell in the BLA. In some embodiments, activating the one or more Ppplrlb⁺-expressing ells comprises activating a hippocampal cell that projects to the one or more Ppplrlb⁺-expressing cells. In some embodiments, the hippocampal cell is a dentate gyrus cell. In certain embodiments, inhibiting the one or more Ppplrlb⁺-expressing cells in the subject, assists in the treatment of a mental disorder or condition in the subject. In some embodiments, the stimulus-activated opsin polypeptide is an excitatory opsin polypeptide. In some embodiments, the stimulus-activated opsin polypeptide is a light-activated opsin polypeptide.

According to yet another aspect of the invention, methods of inhibiting a Rspo2⁺-expressing cell in a subject are provided, the method comprising: expressing in one or more cells in a subject a stimulus-activated opsin polypeptide, wherein the cell in which the stimulus-activated opsin polypeptide is expressed in an Rspo2⁺-expressing cell or in a cell that when inhibited, inhibits an Rspo2⁺-expressing cell in the subject: and activating the expressed stimulus-activated opsin polypeptide; wherein the activation stimulus-activated opsin polypeptide inhibits the Rspo2⁺-expressing cell in the subject. In some embodiments, the Rspo2⁺-expressing cell is a basal lateral amygdala (BLA) cell. In some embodiments, the Rspo2⁺-expressing cell is a magnocellular pyramidal cell in the BLA. In certain embodiments, inhibiting the one or more Rspo2⁺-expressing cells comprises inhibiting a hippocampal cell that projects to the one or more Rspo2⁺-expressing cells. In some embodiments, the hippocampal cell is a dentate gyrus cell. In some embodiments, inhibiting the one or more Rspo2⁺-expressing cells in the subject, assists in the treatment of a mental disorder or condition in the subject. In some embodiments, the stimulus-activated opsin polypeptide is an inhibitory opsin polypeptide. In some embodiments, the stimulus-activated opsin polypeptide is a light-activated opsin polypeptide.

The present invention is not intended to be limited to a system or method that must satisfy one or more of any stated objects or features of the invention. It is also important to note that the present invention is not limited to the exemplary or primary embodiments described herein. Modifications and substitutions by one of ordinary skill in the art are considered to be within the scope of the present invention.

### BRIEF DESCRIPTION OF THE DRAWINGS

FIG. 1A-E illustrates that activating positive memory engrams rescues depression-related behavior. FIG. 1A shows the behavior schedule and groups used. In FIG. 1A, the abbreviation “Dox” is used for “doxycycline,” the female symbols represent exposure to a female conspecific, hexagons represent neutral contexts, and mice in the ‘stress’ condition are depicted undergoing an immobilization protocol. FIG. 1B-1E provide charts showing that optical reactivation of dentate gyrus cells that were previously active during a positive experience significantly increases time struggling in the tail suspension test (shown in FIG. 1B) and preference for sucrose (shown in FIG. 1C) but does not have a significant effect in anxiety-like behavior in the open field test (shown in FIG. 1D) or elevated plus maze test (shown in FIG. 1E). A two-way analysis of variance (ANOVA) with repeated measures revealed a group-by-light epoch interaction in the TST (F_{5,294}=21.20, P<0.001) or SPT (F_{5,196}=6.20, P<0.001) followed by Bonferroni post hoc tests, which revealed significant increases in struggling or preference for sucrose in the positive memory plus stress group. #P<0.01. # used to denote significant differences between the four stressed groups (n=18 per group) versus the two non-stressed groups (n=16 per group); *P<0.05, **P<0.01 (asterisks used to denote significant differences between the stress plus positive memory group versus the other three stressed groups). Data are means±s.e.m.

FIG. 2A-D illustrates that positive memory reactivation increases in c-Fos expression in the nucleus accumbens shell and the amygdala. FIG. 2A shows a brain diagram illustrating target areas analyzed. FIGS. 2B-D show that activation of a positive memory, but not a neutral memory or mCherry only, in the dentate gyrus during the TST elicits robust c-Fos expression in the nucleus accumbens shell (shown in FIG. 2B), basolateral amygdala, and central amygdala (shown in FIG. 2C), but not in the medial prefrontal cortex (shown in FIG. 2D). For histological data, a one-way ANOVA followed by a Bonferroni post hoc test revealed a significant increase of c-Fos expression in the positive memory plus stress group relative to controls in the NAcc and amygdala, but not the mPFC (NAcc shell, F_{2,30}=15.2, P<0.01; BLA, F_{2,30}=11.71, P<0.01; central amygdala, F_{2,30}=11.45, P<0.05; mPFC, F_{2,30}=1.33, P=0.294. n=6 animals per group, 3-5 slices per animal). NS, not significant; *P<0.05, **P<0.01. Data are means s.e.m. Scale bars correspond to 100 μm. HPC, hippocampus; LH, lateral habenula; LS, lateral septum; Hyp., hypothalamus.

FIG. 3A-H illustrates that the antidepressant effects of an optically activated positive memory require real-time terminal activity from the BLA to the NAcc. FIG. 3A shows a brain diagram illustrating target areas manipulated. FIG. 3B illustrates representative coronal slices showing TRE-ArchT-eGFP-positive cells in the BLA or mPFC, as well as their corresponding terminals in the NAcc. Scale bars: BLA and mPFC, 500 mm; NAcc, 200 mm. FIG. 3C shows experimental test results of animals that were taken off Dox and initially exposed to a positive experience, which caused labelling of corresponding BLA (˜18%), mPFC (˜12%), or NAcc (˜9%) cells with eGFP derived from AAV9-TRE-ArchT-eGFP (halo-like expression). Light-activation of a positive memory engram in the dentate gyrus (DG) preferentially reactivated the BLA and NAcc shell cells, as measured by endogenous c-Fos expression (nucleus-localized), that were originally labelled by the same positive experience, while groups with no light stimulation showed levels of overlap not significantly different from chance. Arrowheads indicate double-stained cells. Scale bar, 5 mm. d-g, ArchT-mediated inhibition of BLA, but not mPFC, terminals in the NAcc prevents the dentate-gyms-mediated light-induced increases in struggling (shown in FIGS. 3D and E) or preference for sucrose (shown in FIGS. 3F and G), while inhibition of BLA terminals in the NAcc without dentate gyrus stimulation does not affect behavior (insets). FIG. 3H illustrates that the ArchT-mediated inhibition of BLA, but not mPFC, terminals prevents the dentate-gyms-mediated light-induced increase of c-Fos expression in the NAcc. For behavioral data, a two-way ANOVA with repeated measures followed by a Bonferroni post hoc test revealed a group-by light epoch interaction and significant ArchT-mediated attenuation of struggling in the TST (in FIG. 3D: F_{2,99}=7.30, P<0.001; in FIG. 3E, F_{2,99}=6.61, P<0.01) or preference for sucrose water in the SPT (in FIG. 3F: F_{2,66}=10.66, P<0.01). n=12 per behavioral group. *P<0.05, **P<0.01, ***P<0.001; asterisks used to denote significant differences between the stress plus positive memory group versus all other groups. For histological data, one-sample t-tests against chance overlap were performed (n=4 per group, 3-5 slices per animal). NS, not significant. HPC, hippocampus; LH, lateral habenula; LS, lateral septum; Hyp., hypothalamus. Data are means±s.e.m.

FIG. 4A-D shows that chronic activation of a positive memory elicits a long-lasting rescue of depression-related behavior. FIG. 4A shows a behavioral schedule and groups used. As shown in FIG. 4A, “NoStim” means no stimulation was used, female symbols represent exposure to a female conspecific, hexagons represent neutral contexts, and mice in the ‘stress’ condition are depicted undergoing an immobilization protocol. FIG. 4B illustrates that animals in which a positive memory was reactivated twice a day for 5 days showed increased struggling in a 6-min tail suspension test (F_{5,78}=3.34, P<0.05) and FIG. 4C shows illustrates increased preference for sucrose measured over 24 h (F_{5,84}=6.25, P<0.01). FIG. 4D shows that the 5-day positive memory stimulation group showed a significant increase of adult newborn cells in the dentate gyrus as measured by PSA-NCAM⁺ cells (F_{5,72}=4.65, P<0.01; see FIG. 12 for doublecortin data and PSA-NCAM images). For these data (FIG. 4B-D), a one-way ANOVA revealed a significant interaction of the experimental-group factor and stimulation-condition factor and was followed by a Bonferroni post hoc test. n=14 per TST behavioral group, n=15 per SPT behavioral group, n=5 slices per animal for data appearing in FIG. 4D. *P<0.05. Data are means±s.e.m.

FIG. 5 illustrates that male mice spend more time around an object associated with females. The top of FIG. 5 shows time spent in the target zone where the object associated with females is introduced in the ON phases. Female-object paired mice (experimental group) spend more time in the target zone during the ON phases than the neutral-object paired mice (control group; two-way ANOVA with multiple comparisons, ON 1t₈₈=2.41; P<0.05, ON2 t₈₈=2.08; P<0.05). The bottom of FIG. 5 shows the difference score (average of ON phases−baseline (Bsl)) also shows the increased preference for the target zone in the female-object group compared to neutral-object group (t₂₂=2.37; *P<0.05). n=12 per group. See the Example 1 methods for detailed methods.

FIG. 6A-F shows that positive, neutral, or negative experiences label a similar proportion of dentate gyrus cells with ChR2; stress prevents weight gain over 10 days. FIG. 6A shows c-Fos mice that were bilaterally injected with AAV₉-TRE-ChR2-mCherry and implanted with optical fibres targeting the dentate gyrus. FIG. 6B-E show histological quantifications that reveal that, while off Dox, a similar proportion of dentate gyrus cells are labelled by ChR2mCherry in response to a positive (shown in FIG. 6C), neutral (shown in FIG. 6D), or negative (shown in FIG. 6E) experience. All animals were sacrificed a day after completing the CIS protocol. One-way ANOVA followed by Bonferroni post hoc test, P>0.05, n.s., not significant. In FIG. 6F, animals were chronically immobilized for 10 days, during which they lost a significant amount of weight compared to an unstressed group (one-way ANOVA followed by Bonferroni post hoc test, *P<0.05, n=9 per group). Data are means±s.e.m.

FIG. 7A-B illustrates that reactivation of a positive memory decreases latency to feed in a novelty suppressed feeding paradigm. For the results shown in FIG. 7A, all groups were food deprived for 24 h and then underwent a novelty-suppressed feeding protocol. While chronic immobilization increased the latency to feed, light-reactivation of a positive memory significantly decreased the latency to feed at levels that matched the unstressed groups. For the results shown in FIG. 7B, upon completion of the novelty suppressed feeding test, all groups were returned to their home cage and food intake was measured after 5 min (one-way ANOVA followed by Bonferroni post hoc test, **P<0.01, n=16 per group). Data are means±s.e.m.

FIG. 8A-C shows that the activation of a positive memory elicits BLA spiking activity, requires NAcc glutamatergic activity in the tail suspension test, but does not alter locomotor activity in the open field test. FIG. 8A provides Raster plots and peri-stimulus time histograms (PSTH) illustrating a transient excitatory response from a single BLA neuron out of the nine neurons responsive to dentate gyrus positive memory activation during 10 s of blue light stimulation, but not in response to 10 s of red light as a control. The bar plots on the left illustrate maximum BLA neural firing rate before (Pre) and after (Post) blue light stimulation in the dentate gyrus (paired t-test, t₇=6.91, *P=0.023). The bar plots on the right show the maximum neural activity for the same neurons after red light stimulation in the dentate gyrus that serves as a control (paired t-test, t₇=1.62, P=0.15). FIG. 8B provides a brain diagram illustrating target areas manipulated. Within-subjects experiments revealed that glutamatergic antagonists (Glux), but not saline, in the accumbens shell blocked the light-induced effects of a positive memory in stressed subjects. For behavioral data, a two-way ANOVA with repeated measures followed by a Bonferroni post hoc test revealed a group-by-light epoch interaction on day 1 (F_{1,90}=28.39, P<0.001; n=16 per group) and day 2 of testing (F_{1,90}=8.28, P<0.01). Data are means±s.e.m. As shown in FIG. 8C, all groups failed to show significant changes in locomotor activity within a session of open field exploration during either light off or light on epochs, though any trends towards decreases in locomotion are consistent with stress-induced behavioral impairments. *P<0.05, **P<0.01, ***P<0.001.

FIG. 9A-E illustrates that activating a positive memory in the dentate gyrus produces an increase in c-Fos expression in the lateral septum and hypothalamus, but not the lateral habenula, ventral hippocampus, or VTA. FIG. 9A shows the regions analyzed. FIG. 9B shows that c-Fos expression significantly increased in the lateral septum and subregions of the hypothalamus including the dorsomedial (DM), ventromedial (VM), and lateral hypothalamus. FIG. 9C-E show that c-Fos expression did not significantly increase in the lateral habenula (illustrated in FIG. 9C), various ventral hippocampus subregions (illustrated in FIG. 9D), or VTA, identified by tyrosine hydroxylase staining in the images expanded on the right (illustrated in FIG. 9E) (one-way ANOVA followed by Bonferroni post hoc test *P<0.05, **P<0.01, n=5 animals per group, 3-5 slices per animal). TS, tail suspension. Data are means±s.e.m.

FIG. 10A-B illustrates that activating a positive memory through the dentate gyrus of unstressed animals increases c-Fos expression in various downstream regions. FIG. 10A provides a diagram of regions analyzed. FIG. 10B shows that in the positive compared to the neutral memory group, c-Fos expression is significantly increased in the lateral septum, NAcc shell, BLA, dorsomedial, ventromedial and lateral hypothalamus, but not in the mPFC, NAcc core, habenula, or ventral hippocampus. Trends were observed in the central amygdala (CeA) and VTA. Each brain region was analyzed using an unpaired student's t test, n=5 animals per group, 3-5 slices per animal; #P=0.17 for central amygdala and P=0.09 for VTA; *P<0.05, **P<0.01, ***P<0.001, n.s., not significant. Data are means±s.e.m.

FIGS. 11A and B shows that dopamine receptor antagonists block the light-induced effects of positive memory activation; a single session of activating a positive memory in the dentate gyrus does not produce long-lasting antidepressant-like effects. The experimental results shown in FIG. 11A indicate that the administration of a cocktail of dopamine receptor antagonists (DAx) prevented the light-induced increases in struggling during the tail suspension test. When animals were tested again on day 2 and infused with saline, the behavioral effects of optically reactivating a positive memory were observed (two-way ANOVA with repeated measures followed by Bonferroni post hoc test, *P<0.05, n=9 per group). The experimental results shown in FIG. 11B indicate that animals in which a positive memory was optically activated during the tail suspension test or sucrose preference test showed acute increases in time struggling or preference for sucrose; this change in behavior did not persist when tested again on day 2 (within subjects ANOVA followed by Bonferroni post hoc test), n=9. n.s., not significant. Data are means±s.e.m.

FIG. 12A-M shows that chronic activation of a positive memory prevents stress-induced decreases in neurogenesis. As shown in FIG. 12A, the 5-day positive memory stimulation group showed a significant increase of adult newborn cells in the dentate gyrus as measured by doublecortin (DCX)-positive cells (one-way ANOVA followed by Bonferroni post hoc test, F_{5,72}=7.634, P<0.01) relative to control groups. FIG. 12B-G show representative images of DCX-positive cells in the dentate gyrus for the 5-day (shown in FIG. 12B), 1-day (shown in FIG. 12C), neutral (shown in FIG. 12D), no stimulation (shown in FIG. 12E), natural (shown in FIG. 12F), and no stress (shown in FIG. 12G) groups. FIG. 12H-M provides representative PSA-NCAM images corresponding to data appearing in FIG. 4D. n=5 slices per animal, 13 animals per group for data appearing in FIG. 12A. *P<0.05, n.s., not significant. Data are means±s.e.m.

FIGS. 13A and B illustrates behavioral and neuronal correlations. FIG. 13A shows that performance levels in the SPT and the number of adult-born neurons as measured by PSA-NCAM are positively correlated on an animal-by-animal basis. FIG. 13B shows that performance levels between the TST and SPT show strong positive correlation trends on an animal-by-animal basis. n=14 per TST behavioral group, n=15 per SPT behavioral group.

FIG. 14A-L shows activity-dependent transcriptional profiling of BLA neurons. FIG. 14A, viral-based genetic scheme for activity-dependent transcriptional profiling. c-Fos promoter activity drives the expression of tTA, which in turn, binds TRE and drives the expression of PABP-FLAG in the absence of doxycycline (Dox). FIG. 14B, PABP-FLAG expression in the BLA in mice kept on a Dox diet (On Dox), taken off a Dox diet and exposed to home cage (Off Dox), Shock, Female, Seizure, (one-way ANOVA, P<0.0001, n=6 per group). Significance for multiple comparisons, **P<0.01, ****P<0.0001, not significant (N.S.). FIG. 14C, PABP-FLAG expression in soma and varicosities of a BLA neuron. FLAG expression in the BLA of On Dox (Dig. 14D), Off Dox (FIG. 14E), Seizure (FIG. 14F), Shock (FIGS. 14G,H), and Female (FIGS. 14I,J) group. FLAG expression and nuclear marker, DAPI, in Shock (FIG. 14H), and Female (FIG. 14J) group. Scale bar 25 μm (FIG. 14C), 250 μm (FIGS. 14D,E,F,G,I), 80 μm (FIGS. 14H,J). FIG. 14K, RMA normalized RNA expression values from microarray from RNA purified from Shock (n=3) and Female (n=3) groups. The points represent enriched genes (>1.25 fold, ANOVA p≤0.05, log 2 scale). FIG. 14L, Quantification of in situ hybridization of BLA expression of candidate genetic markers enriched in shock group (no shading) and female group (diagonal line shading) (n=3 mice per group). Positive control genes (black). Results show mean±s.e.m (FIGS. 14B,L).

FIG. 15A-K shows Rspo2⁺ and Ppplrlb⁺ BLA neurons define spatially segregated populations of BLA pyramidal neurons. FIG. 14A, Quantification of smFISH of Rspo2 and Ppplrlb expression across the AP axis (coronal distance from bregma −0.8 mm to −2.8 mm) of the BLA (n=3). FIG. 15B, Two sagittal views (ML distance from midline, 3.2 mm, 3.4 mm) of double smFISH of Rspo2 and Ppplrlb with nuclear marker, DAPI, in the BLA FIG. 15C, Coronal view of double smFISH of Rspo2 and Ppplrlb across the AP axis of the BLA. Double smFISH of Camk2a and Rspo2 (FIG. 15D), Camk2a and Ppplrlb (FIG. 15E), Gad1 and Rspo2 (FIG. 15F) Gad1 and Ppplrlb (FIG. 15G), in the BLA (Larger micrograph in FIG. 22). Scale bar 500 μm (FIG. 15B), 200 μm (FIG. 15C), 25 μm (FIG. 15D-G). FIG. 15H, Biocytin filled magnocellular (top) and parvocellular (bottom) BLA neuron, scale bar 50 μm. FIG. 15I, Single-cell qPCR traces of Rpso2 and Ppplrlb, of magnocellular (top) and parvocellular (bottom) BLA neurons. FIG. 15J, Electrophysiological response to current steps in a Rspo2⁺ (top) and Ppplrlb⁺ (bottom) BLA neuron. FIG. 15K, Comparison of mean soma diameter, membrane resistance (Rm), and membrane capacitance (Cm) of qPCR-confirmed Rpso2⁺ (n=11) and Ppplrlb⁺ (n=12) neurons. Significance for unpaired t-test, **P<0.01, ***P<0.001, ****P<0.0001. Results show mean±s.e.m (FIGS. 15A,K).

FIG. 16A-R illustrates that Rspo2⁺ and Ppplrlb⁺ BLA neurons are activated by valence-specific stimuli. c-FOS expression across the AP axis (coronal distance from bregma −0.8 mm to −2.8 mm) of the BLA in response to shock (n=8), context (n=8), female (n=6) (FIG. 16A); TMT (n=6), BA (n=7), peanut oil (n=6) (FIG. 16B); quinine water (n=8), no water (n=8), water (n=6), sucrose water (n=8) (FIG. 16C). The total number of c-FOS+ cells is represented for each coronal section of a unilateral BLA (a-c), micrographs found in FIG. 23. FIG. 16D, Relative c-FOS expression in the aBLA and pBLA in response to shock, context, female (one-way ANOVA, P<0.0001). FIG. 16E, Relative c-FOS expression in response to TMT, BA, peanut oil (one-way ANOVA, P=0.0001,). FIG. 16F, Relative c-FOS expression in response to quinine water, no water, water, sucrose water (one-way ANOVA, P<0.0001). Significance for multiple comparisons (FIG. 16D-F), *P<0.05, **P<0.01, ****P<0.0001, not significant (N.S.). Double-label smFISH (n=5 in each group) of c-Fos/Rspo2⁺ (FIGS. 16G,K,M) or c-Fos/Ppplr1b⁺ (FIGS. 16H,L,N) in response to shock (S) or context (C). Double-label smFISH of c-Fos−/Rspo2⁺ (FIGS. 16I,O,Q) or c-Fos/Ppplrlb+(FIGS. 16J,P,R) in response to water (W) or no water (NW). Significance for unpaired t-test (FIG. 16G-J), **P<0.01, not significant (N.S). Scale bar 125 μm (FIG. 16K-R). Results show mean±s.e.m (FIG. 16A-J).

FIG. 17A-S illustrates that Rspo2⁺ and Ppplrlb⁺ BLA neurons participate in valence-specific behaviors. FIG. 17A, Optogenetically targeting Rspo2⁺ and Ppplrlb⁺ BLA neurons. Scheme and results for Rspo2-Arch and Ppplrlb-Arch mice in a fear (FIG. 17B,C) and reward (FIGS. 17D,E) conditioning. FIG. 17C, Rspo2-Arch mice (n=9) displayed lower freezing on Day 1 and 2 compared to eYFP controls (n=8), no difference between Ppplrlb-Arch (n=8) and Ppplrlb-eYFP (n=6) mice. FIG. 17E, Ppplrlb-Arch mice (n=10) displayed lower total nose pokes and cue-reward association in nose port (z-score) compared to EYFP controls (n=11), no difference between Rspo2-Arch (n=9) and Rspo2-eYFP (n=8). Scheme and results for Rspo2-ChR2 and Ppplrlb-ChR2 mice in an optogenetic freezing test (FIGS. 17F,G), optogenetic self-stimulation test (FIGS. 17H,I), and optogenetic place preference test (FIGS. 17J,K). g, Rspo2-ChR2 mice (n=7) displayed greater freezing levels on Day 1 and 2 compared to EYFP controls (n=6), no difference between Ppplrlb-ChR2 (n=5) and Ppplrlb-eYFP (n=5) mice. FIG. 17I, Ppplrlb-ChR2 mice (n=6) displayed greater levels of nose pokes on Day 1 and 2 compared to EYFP controls (n=6), no difference between Rspo2-ChR2 (n=8) and Rspo2− eYFP (n=6) mice. FIG. 17K, Rspo2-ChR2 mice (n=11) displayed greater preference to light stimulation compared to eYFP controls (n=8), while Ppplrlb-ChR2 (n=7) mice displayed greater preference to light stimulation compared to eYFP controls (n=7). Scheme and results for activating BLA neurons in Rspo2-ChR2 and Ppplrlb-ChR2 mice during shocks (FIGS. 17L,M), or water consumption (FIGS. 17N,O). FIG. 17M, Ppplrlb-ChR2 (n=8) displayed lower freezing levels compared to EYFP controls (n=8), no difference between Rspo2-ChR2 (n=6) and Rspo2-eYFP (n=6) mice. FIG. 17O, Rspo2-ChR2 mice (n=6) displayed lower total nose pokes and cue-reward association compared to EYFP controls (n=5), no difference between Ppplrlb-ChR2 (n=9) and Ppplrlb614 eYFP (n=7) mice. Significance for unpaired t-test between experimental groups compared to corresponding EYFP controls, *P<0.05, **P<0.01, ***P<0.001, ****P<0.0001, not significant (N.S), results show mean±s.e.m (FIGS. 17C,E,G,I,K,M,O). Expression of eArch-EYFP in Rspo2-Arch mice (FIG. 17P) and Ppplrlb-Arch mice (FIG. 17Q). Expression of ChR2-EYFP across the AP axis of the BLA in Rspo2-ChR2 mice (FIG. 17R) and Ppplrlb-ChR2 mice (FIG. 17S). Strong Ppplrlb⁺ fibers are found in the central amygdala (FIGS. 17Q,S). Scale bar, 300 μm (FIGS. 17P,Q,R,S).

FIG. 18A-P illustrates how Rspo2⁺ and Ppplrlb⁺ BLA neurons project to distinct amygdaloid nuclei and prefrontal areas. Quantification of CTB⁺ neurons across the AP axis (coronal distance from bregma −0.8 mm to −2.8 mm) of the BLA from CTB targeted to the amygdala and extended amygdala areas (FIG. 18A)—CeC (FIGS. 18C,D), CeL/CeM (FIGS. 18E,F), NAc (FIGS. 18G,H), or dual CTB targeted to prefrontal cortex (FIG. 18B)—PL and IL (FIGS. 18I,J) (n=3 per group). Injections site of CTB (FIGS. 18C,E,G,I) and CTB⁺ BLA neurons (FIGS. 18D,F,H,J). Co-labelling of Rspo2 mRNA in the BLA with CTB targeted to the CeC (FIG. 18K) and NAc (FIG. 18M). Co-labelling of Ppplrlb mRNA in the BLA with CTB injected into the CeL/CeM (FIG. 18L) and NAc (FIG. 18N), quantification in Data Table 1, micrographs in FIG. 26. Rspo2− ChR2+ fibers are found in the CeC, NAc, and PL (FIG. 18B). Ppplrlb-ChR2+ fibers are found in the CeL, CeM, NAc, and IL (FIG. 18P). Scale bar 250 μm (FIG. 18C-J,O,P), 25 μm (FIG. 18K-N). Results show mean±s.e.m.

FIG. 19A-N illustrates that Rspo2⁺ and Ppplrlb⁺ BLA neurons establish reciprocal inhibitory connections. FIGS. 19A,B, Scheme for the experimental setup for recording in magnocellular (Rspo2⁺) (FIG. 19A) and parvocellular (Ppplrlb⁺ (FIG. 19B) neurons, while stimulating Ppplrlb⁺ (Ppplrlb-ChR2 mice) and Rpso2⁺ (Rspo2-ChR2 mice) neurons, respectively. FIGS. 19C,D Sagittal view of biocytin-filled magnocellular BLA neurons in Ppplrlb-ChR2 mice (FIG. 19C) and parvocellular BLA neurons in Rspo2-ChR2 mice (FIG. 19D). Scale bar 200 μm, inset: 50 μm (FIGS. 19C,D). Asterisks denote the electrophysiological traces in FIG. 19E and FIG. 19F. Inhibitory postsynaptic potentials (IPSPs) recorded in magnocellular (FIG. 19E) and parvocellular (FIG. 19F) BLA neurons by 10 Hz optogenetic stimulation of Ppplrlb-ChR2 (FIG. 19E) and Rspo2-ChR2 (FIG. 19F) fibers. Traces shown in FIGS. 19E and F represent average trace of 20 sweeps recorded during periods without spikes. Inhibitory postsynaptic currents (IPSCs) recorded in magnocellular (FIG. 19G) and parvocellular (FIG. 19H) BLA neurons (clamped at 0 mV) in response to optogenetic stimulation (10 Hz train) of Ppplrlb-ChR2⁺ (FIG. 19G) and Rspo2-ChR2⁺ (FIG. 19H) fibers. Currents are blocked by bath application of gabazine (GBZ, 10 μM), insets: IPSCs amplitude before (GBZ⁻) and after GBZ (GBZ⁺) application for both magnocellular (n=6) (FIG. 19G) and parvocellular (n=6) (FIG. 19H), Wilcoxon signed-rank test, *P<0.05. Probability of connection, parvocellular to magnocellular connection (FIG. 19I) and magnocellular to parvocellular connection (FIG. 19J). The two groups interact predominately by mutual inhibition rather than excitation, Fisher exact test, ***P<0.001 (FIGS. 19I,J). IPSC onset in magnocellular (left) and parvocellular (right) neurons were similar (FIG. 19K). IPSC amplitude was greater in parvocellular (right) than in magnocellular (left) neurons (FIG. 19L), unpaired two-tailed paired t-test *P<0.05. Recorded magnocellular and parvocellular neurons were confirmed using soma diameter and anatomical position (FIG. 19M); membrane resistance (Rm) and membrane capacitance (Cm) (FIG. 19N). Magnocellular and parvocellular cells were statistically distinct in all four parameters and consistent with values characterized in FIG. 15, significance for unpaired two-tailed paired t-test *P<0.05, **P<0.01, ****P<0.0001 (n, m). Results show mean±s.e.m (FIGS. 19G,H,K,L).

FIG. 20A-B provides traces illustrating RNA analysis of activity-dependent transcriptional profiles from BLA neurons. FIG. 20A, Example bioanalyzer traces of RNA samples collected from footshock (middle trace) (n=3), female (top trace) (n=3), on dox (bottom trace) group (n=1). Bioanalyzer traces was used to test the quality of RNA sample for RNA microarray, the graph shows the fluorescence levels, which corresponds to RNA levels, of different RNA species of different size (nt). Bioanalyzer traces showed that footshock and female samples yielded RNA samples with RNA quality number (RQN)>6 (n=6), while the on dox RNA sample RQN<4 (n=1). Peaks at 0.02 kb, 1.9 kb and 4.7 kb correspond to the marker, 18S rRNAs, and 28S rRNAs, respectively. FIG. 20B, Analysis of MAS5 normalized data of arrays from the footshock (n=3) and female (n=3) group.

FIG. 21A-V provides photomicrographic images showing In situ hybridization of candidate genetic markers of BLA neurons. Gene expression of candidate genetic markers in the BLA using in situ hybridization. FIG. 21A-G, Genes that were enriched in the array of the footshock group. FIG. 21H0-P, Genes that were enriched in the array of the female group. FIG. 21Q-T, Positive control for interneurons. FIGS. 21U,V Positive control for excitatory neurons (yellow). Micrographs represent FISH with the exception of Ppplrlb (smFISH). FIG. 21A-V, nuclear marker, DAPI. Scale bar 100 μm.

FIG. 22A-F provides photomicrographic images showing Rspo2⁺ and Ppplrlb⁺ BLA neurons collectively constitute all BLA pyramidal neurons. smFISH of Rpso2/Camk2a (FIG. 22A), Rspo2/Gad1 (FIG. 22B), Ppplrlb/Camk2a (FIG. 22C), Ppplrlb/Gad1 (FIG. 22D), coronal BLA, scale bar 200 μm. FIG. 22E, smFISH of Rpso2+Ppplrlb/Camk2a, sagittal BLA, scale bar 250 μm. FIG. 22F, higher magnification expression of Rpso2+ Ppplrlb/Camk2a, scale bar 50 μm.

FIG. 23A-C shows immunostained tissues demonstrating spatial distribution of C-FOS expression in the BLA in response to valence-specific stimuli. C-FOS protein was visualized using IHC by an Alexa Fluor 555 secondary antibody. For improved graphical representation, images were inverted and saturation removed. FIG. 23A, C-FOS expression across the AP-axis of the BLA in response to shock, context, female. FIG. 23B, C-FOS expression across the AP-axis of the BLA in response to olfactory stimuli. FIG. 23C, C-FOS expression across the AP-axis of the BLA in response to gustatory stimuli. Scale bar 250 μm.

FIG. 24A-D provides graphs and photomicrographic images showing validation of Cre-driver mouse lines for targeting Rspo2⁺ and Ppplrlb⁺ BLA neurons. Rpso2-Cre and Cartpt-Cre mice were injected with a Cre-dependent eYFP virus into the BLA and smFISH was performed against Rspo2 and Ppplrlb, respectively. FIG. 24A, Quantification of the percentage of Rpso2⁺ BLA neurons that express eYFP (eYFP/Rspo2) and the percentage of eYFP⁺ BLA neurons that express Rspo2 (Rpso2/eYFP) (n=4). FIG. 24B, eYFP and Rspo2 expression in the BLA of virus injected Rspo2-Cre mice. FIG. 24C, Quantification of the percentage of Ppplrlb⁺ BLA neurons that express eYFP (eYFP/Ppplrlb) and the percentage of eYFP⁺ BLA neurons that express Ppplrlb (Ppplrlb/eYFP) (n=4). FIG. 24D, eYFP and Ppplrlb expression in the BLA of virus injected Cartpt-Cre mice. Although Ppplrlb is endogenously expressed not only in pBLA, but also in some cells outside of the BLA, such as in the intercalated cell mass, choroid plexus, and striatum (FIG. 2C), Credependent virus targeted in the Cartpt-Cre mice permitted largely pBLA-restricted expression of Pppllr1b. Scale bar 250 μm.

FIG. 25A-B provides photomicrographic images showing fiber placement for targeting Rspo2⁺ and Ppplrlb⁺ BLA neurons. Example of optic fiber placement in Rspo2-Arch (FIG. 25B) and Ppplrlb-Arch (FIG. 25B) mice. Scale bar, 500 μm.

FIG. 26A-F provides photomicrographs of retrograde tracing from putative projection targets of Rspo2⁺ and Ppplrlb⁺ BLA neurons. smFISH of Rspo2 and Ppplrlb in CTB injected brains. Rspo2 (FIG. 26A) and Ppplrlb (FIG. 26B) expression in the BLA of CeC-CTB mice. Rspo2 (FIG. 26C) and Ppplrlb (FIG. 26D) expression in the BLA of CeL/M-CTB mice. Rspo2 (FIG. 26E) and Ppplrlb (FIG. 26F) expression in the BLA of NAc-CTB mice. Scale bar 250 μm.

FIG. 27A-D provides graphs and images showing activation of NAc fibers of Rspo2⁺ BLA neurons elicits negative behaviors. Optic fiber was unilaterally implanted above the NAc of Rspo-ChR2 mice (NAc Rpso2− ChR2). NAc Rspo2-ChR2 underwent behavioral assays. FIG. 27A, Optogenetic freezing test (n=9). FIG. 27B, Optogenetic self-stimulation test (n=11). FIG. 27C, Optogenetic place preference test (n=9). Behavioral performance was compared against Rspo2-ChR2 (FIG. 4) using an unpaired t-test. No significant difference was observed across all assays. FIG. 27D, Optic fiber placement in the NAc of Rspo2-ChR2 mice. Scale bar 500 μm.

FIG. 28A-C provides schematic diagrams showing a circuit model of the BLA. FIG. 28A, Anatomical connections of genetically identifiable population of amygdala neurons. Projections identified, but cell-type unknown*, hypothetical**. FIG. 28B, The negative circuit of the amygdala. CeC and PL projections are key distinguishing features of Rspo2⁺ BLA neurons from Ppplrlb⁺ BLA neurons. Rspo2⁺ BLA neurons project the CeC, but the genetic identity of the neurons that are innervated has yet to be identified; one possibility is CeL Calcrl⁺ neurons. Nevertheless, if Rspo2⁺ BLA neurons ultimately activate the effector neurons of freezing in the CeM, then an indirect route must be taken through the CeC and/or possibility the intercalated cell (not depicted). FIG. 28C, The positive circuit of the amygdala. CeL, CeM, and IL projections are distinguishing features from Ppplrlb⁺ BLA neurons to Rspo2⁺ BLA neurons. Ppplrlb⁺ BLA neurons send dense fibers to the CeL and CeM. Therefore, a population in the CeL and/or CeM may mediate appetitive behaviors; possibly, CeM Tact− neurons.

### DETAILED DESCRIPTION

The invention relates, in part, to methods to rescue stress-induced depression-related behaviors by optogenetically reactivating dentate gyrus cells that were previously active during a positive experience. A brain-wide histological investigation, coupled with pharmacological and projection-specific optogenetic blockade experiments, identified glutamatergic activity in the hippocampusamygdalanucleus-accumbens pathway as a candidate circuit supporting the acute rescue. Chronically reactivating hippocampal cells associated with a positive memory has now been show to result in the rescue of stress-induced behavioral impairments and to result neurogenesis at time points beyond the light stimulation. It has now been demonstrated that activating positive memories artificially is sufficient to suppress depression-like behaviors, depression, post-traumatic stress disorder (PTSD), etc. and indicate dentate gyrus engram cells as therapeutic nodes for intervening with maladaptive behavioral states.

Negative and positive emotional stimuli elicit opposing behaviors. The basolateral amygdala (BLA) is a site of convergence of negative and positive stimuli, and is involved in emotional behaviors and associations. A neural substrate for negative and positive behaviors and neural circuit that underlies the antagonistic nature of negative and positive behaviors in the basolateral amygdala has now been identified and can be used in methods of the invention to condition a positive or negative behavior in a subject to a neutral environmental context.

Two genetically distinct, spatially segregated populations of excitatory neurons in BLA have now been identified that participate in valence-specific behaviors and memories. R-spondin 2-expressing (Rspo2+) neurons, which define the anterior BLA, are necessary and sufficient for fear-related behaviors and memories. Protein phosphatase 1 regulatory subunit 1B-expressing (Ppplrlb⁺) neurons, which define the posterior BLA, are necessary and sufficient for reward-related behaviors and memories. Activation of Rspo2⁺ neurons during a rewarding stimulus reduces appetitive behaviors, while activation of Ppplrlb⁺ neurons during a threatening stimulus reduces defensive behaviors and memories. Rspo2⁺ and Ppplrlb⁺ neurons interact through reciprocal inhibitory connections. Studies have now identified neurons and a neural circuit for control of antagonistic emotional behaviors, and methods of the invention, in part, include use of optogenetics to modulate (increase or decrease) activation of one or more of newly identified types of neuron populations to alter behavior and in treatments for various mental disorders and other conditions.

The invention, in part, relates to methods to treat a mental disorder or other mental condition in a subject. In addition, compositions that include compounds useful to treat a mental disorder or a condition are also provided in aspects of the invention. Compounds of the invention include polypeptide and/or polynucleotide molecules. For example, a compound may comprise a polynucleotide that encodes a polypeptide compound. Methods of the invention, in some embodiments include expression of fusion proteins that comprise a stimulus-activated opsin polypeptide such as a stimulus-activated ion-channel polypeptide or a stimulus-activated ion pump polypeptide. In some embodiments of the invention, a fusion protein comprises a stimulus-activated opsin polypeptide and one or more of a detectable label polypeptide, a trafficking polypeptide, a targeting polypeptide, or other polypeptide that is of interest to express in the cell in which the fusion protein is expressed.

It has now been determined that the reactivation of a positive memory engram in a subject can assist in the treatment of a mental disorder or condition in a subject. Certain embodiments of methods of the invention include creating a positive memory engram that can be re-actived using methods of the invention to treat a mental disease or condition. The term “engram” is a term used in the art in reference to a means by which memories are stored. Formation of engrams may include activation of neurons during the process of acquiring a memory, and resulting lasting physical or chemical changes. An engram may include encoding in neural tissue that provides a physical basis for the persistence of memory. Other aspects of the invention include re-activion of an existing positive memory engram in a treatment method for a mental disorder or condition. In addition, specific cell types have now been identified that can be modulated to specifically alter behavioral responses.

Different types of stimulus-activated opsin molecules (polypeptides and/or encoding polynucleotides) are known in the art and may be suitable for use in embodiments of the invention. Examples of stimulus-activated opsins that may be include in a composition of the invention and may be expressed in a subject as part of a treatment method of the invention, are channelrhodopsin, halorhodopsin, Archaerhodopsin, and Leptosphaeria rhodopsin polypeptides and their encoding polynucleotides. Many stimulus-activated opsin molecules are known in the art and have been used to alter membrane potential in electrically excitable cells. Stimulus-activated opsin molecules are routinely expressed in fusion proteins and used in optogenetic methods and compositions. Expression of such an opsin in a cell permits modulation of the cell's membrane potential when the cell is contacted with a suitable light, or other stimulatory means. Methods to prepare and express a light-activated opsin in a cell, and in a subject, are well known in the art, as are methods to select and apply a suitable wavelength of light to the cell in which the opsin is expressed in order to activate the expressed opsin ion channel or ion pump in the cell. Methods of adjusting illumination variables and conditions for activation are well-known in the art and representative methods can be found in publications such as: Lin, J., et al., Biophys. J. 2009 Mar. 4; 96(5):1803-14; Wang, H., et al., 2007 Proc Natl Acad Sci USA. 2007 May 8; 104(19):8143-8. Epub 2007 May 1; the content of each of which is incorporated herein by reference in its entirety. It will be understood that an opsin polypeptide that is activated or inhibited by light or that is activated or inhibited by another stimulation means can be used in aspects of compositions and methods of the invention.

In certain implementations, the invention comprises methods for preparing and using genes encoding stimulus-activated opsins such as light-activated ion channel polypeptides and light-activated ion pumps in vectors that may also include additional polynucleotide molecules that encode trafficking polypeptides, detectable labels, or other molecules of interest to be expressed in a cell with the opsin polypeptide. Some embodiments of the invention include expression in cells, tissues, and subjects of one or more opsin polypeptides.

As used herein, the terms “opsin polypeptide” and “opsin amino acid sequence” when used in reference to an opsin molecule that is included in a composition or method of the invention, means an opsin polypeptide or a functional variant thereof, and an amino acid sequence of an opsin, or functional variant thereof. Similarly, the terms “opsin polynucleotide” and “opsin nucleic acid sequence” when used in reference to an opsin molecule that is included in a composition or method of the invention, means an opsin polynucleotide or a functional variant thereof, and a nucleic acid sequence encoding an opsin or a functional variant thereof. Certain embodiments of compositions, compounds, and methods of the invention may additionally include a vector or construct that comprises such polynucleotides or nucleic acid sequences.

### Sequences and Functional Variants

The term “variant” as used herein in the context of polypeptide molecules and/or polynucleotide molecules, describes a molecule with one or more of the following characteristics: (1) the variant differs in sequence from the molecule of which it is a variant, (2) the variant is a fragment of the molecule of which it is a variant and is identical in sequence to the fragment of which it is a variant, and/or (3) the variant is a fragment and differs in sequence from the fragment of the molecule of which it is a variant. As used herein, the term “parent” in reference to a sequence means a sequence from which a variant originates. For example, though not intended to be limiting: a ChR2 sequence is the parent sequence for ChR2 functional variant.

An opsin molecule that is a functional variant of a wild-type or other opsin molecule may have all or part of the sequence of its parent molecule, but with a change or modification of 1, 2, 3, 4, 5, 6, 7, 8, 9, 10, 11, 12, 13, 14, 15, 16, 17, 18, 18, 20, 21, 22, 23, 24, 25, or more amino acids or nucleic acids, compared to its parent amino acid or nucleic acid sequence, respectively. As used herein, a sequence change or modification may be one or more of a substitution, deletion, insertion or a combination thereof. Opsin molecule and variants thereof are known in the art and may be included in compositions, compounds of the invention, and may be used in methods of the invention. Standard art-known methods can be used to identify, select, and/or use an opsin polypeptide or a functional variant thereof and its encoding nucleic acid sequence.

As used herein an amino acid sequence of an opsin polypeptide variant may have 85% 90%, 95%, 96%, 97%, 98% 99% sequence identity to its parent amino acid sequence. As used herein a nucleic acid sequence encoding an opsin polypeptide variant may have 85% 90%, 95%, 96%, 97%, 98% 99% sequence identity to its parent nucleic acid sequence. Routine sequence alignment methods and techniques can be used to align two or more similar light-activated opsin polypeptide sequences, including but not limited to wild-type and previously identified opsin polypeptide sequences, thus providing a means by which a corresponding location of a modification made in one opsin polypeptide can be identified in opsin polypeptide sequence. Such sequence alignment means can also be performed to align and identify variants from parent polynucleotide sequences.

It is understood in the art that the codon systems in different organisms can be slightly different, and that therefore where the expression of a given protein from a given organism is desired, the nucleic acid sequence can be modified for expression within that organism. Thus, in some embodiments, an opsin and/or fusion protein of the invention is encoded by a mammalian-codon-optimized nucleic acid sequence, which may in some embodiments be a human-codon optimized nucleic acid sequence. In certain aspects of the invention, a nucleic acid sequence used in a compound, composition, or method of the invention is a sequence that is optimized for expression in a human cell.

As used herein, the terms “protein” and “polypeptide” are used interchangeably and thus the term polypeptide may be used to refer to a full-length protein and may also be used to refer to a fragment of a full-length protein, and/or functional variants thereof. As used herein, the terms “polynucleotide” and “nucleic acid sequence” may be used interchangeably and may comprise genetic material including, but not limited to: RNA, DNA, mRNA, cDNA, etc., which may include full length sequences, functional variants, and/or fragments thereof.

### Fusion Protein Components

Certain embodiments of a pharmaceutical composition of the invention include a fusion protein or a molecule encoding a fusion protein. Molecules that can be expressed in a fusion protein and used in an embodiment of a treatment method of the invention may include one or more: opsin polypeptides, detectable label polypeptides, targeting polypeptides, and trafficking polypeptides, etc. Non-limiting examples of detectable label polypeptides include: green fluorescent protein (GFP); enhanced green fluorescent protein (EGFP), red fluorescent protein (RFP); yellow fluorescent protein (YFP), dtTomato, mCherry, DsRed, cyan fluorescent protein (CFP); far red fluorescent proteins, etc. Numerous fluorescent proteins and their encoding nucleic acid sequences are known in the art and routine methods can be used to include such sequences in fusion proteins and vectors, respectively, of the invention.

Additional sequences that may be included in a fusion protein of the invention are trafficking, also referred to as “export” sequences, including, but not limited to: Kir2.1 sequences and functional variants thereof, KGC sequences, ER2 sequences, etc. Additional trafficking polypeptides and their encoding nucleic acid sequences are known in the art and routine methods can be used to include and use such sequences in fusion proteins and vectors, respectively, of the invention.

Compositions and compounds for use in certain embodiments of treatment methods of the invention also include one or more opsin molecules. As used herein, the term “opsin” includes stimulus-activated opsin molecules that when expressed in a cell and contacted with a suitable stimulus, function as a membrane channel, an ion pump, or other identified structure, based on its sequence. A stimulus-activated opsin may be an “excitatory” or “activating” when activated or may be “inhibitory” when activated. A non-limiting example of an excitatory stimulus-activated opsin is an ion-channel opsin that when activated results in depolarization of the cell in which it is expressed. A non-limiting example of an inhibitor stimulus-activated opsin is an opsin that when activated results in hyperpolarization of the cell in which it is expressed.

A non-limiting example of an opsin useful in certain compositions and methods of the invention is a light-activated opsin. As used herein the term “opsin” may include any opsin having a sequence that is one or more of: a wild type opsin sequence, a modified opsin sequence, a mutated opsin sequence, a chimeric opsin sequence, a synthetic opsin sequence, a functional fragment of an opsin sequence that may include one or more additions, deletions, substitutions, or other modifications to the sequence of the parent opsin sequence from which the fragment sequence originates, and a functional variant of an opsin sequence that may include one or more additions, deletions, substitutions, or other modifications to the sequence of the parent opsin sequence from which the variant sequence originates. As used herein the term “functional” when used in reference to a fragment or variant means that the fragment or variant retains at least a portion of a function of the parent molecule. For example, a functional variant of a light-activated ion channel polypeptide differs from its parent sequence and retains at least some of the light-activated ion channel activity of its parent.

Methods of preparing and using opsin molecules and functional variants thereof are well known in the art and such opsins may be used in aspects of the invention. Examples of categories of opsin molecules, whose members may be included in compositions of the invention and used in methods of the invention include, but are not limited to light-activated microbial opsins such as halorhodopsins, channelrhodopsins, Archaerhodopsins, and Leptosphaeria rhodopsins, members of each of which are well known in the art. Non-limiting examples of opsins that may be included embodiments of compositions, vectors, and used in methods of the invention are: CoChR, ChR2, ChR88, ChR90, ChR64, ChR86, ChR87, ChR90, Chrimson, ChrimsonR, Chronos, CsChrimson, ReaChR, GtACR, SwiChRca, iChloC, ChloC, ChIEF, V1C1, ChR2-2A-Halo, VChR1, Halo57, Jaws, Halo (also known as: NpHR), eNpH; R, eNpHR 3.0, Arch, eArch 3.0, ArchT, ArchT 3.0, Mac, Mac 3.0, and functional mutants (also referred to as “functional variants” thereof [see Klapoetke et al. (2014) Nature Methods 11(3), 338-346; for review see: Yizhar, O. et al. (2011) Neuron Vol. 71:9-34; the content of each of which is incorporated by reference herein in its entirety.] Additional opsin polypeptides and their encoding nucleic acid sequences are known in the art and routine methods can be used to include and use such sequences and functional variants thereof in fusion proteins and vectors, respectively, of the invention.

### Delivery of Polypeptides

Delivery of an opsin molecule to a cell and/or expression of an opsin polypeptide in a cell can be done using art-known delivery means. In some embodiments of the invention a trafficking polypeptide and an opsin polypeptide are included in a fusion protein. It is well known in the art how to prepare and utilize fusion proteins that comprise one or more polypeptide sequences. In certain embodiments of the invention, a fusion protein can be used to deliver an opsin polypeptide, such as a stimulus-activated opsin polypeptide or functional variant thereof of the invention to a cell as part of a treatment method of the invention. A fusion protein for use in methods of the invention can be expressed in a specific cell type, tissue type, organ type, and/or region in a subject, or in vitro, for example in culture, in a slice preparation, etc. Preparation, delivery, and use of a fusion protein and its encoding nucleic acid sequences are well known in the art. Routine methods can be used in conjunction with teaching herein to express a fusion protein comprising an opsin polypeptide in a desired cell, tissue, or region in vitro or in a subject. Methods suitable to deliver fusion proteins into cells are presented herein and various methods are described in the art, [see Klapoetke et al. (2014) Nature Methods 11(3), 338-346; for review see: Yizhar, O. et al. (2011) Neuron Vol. 71:9-34].

It is an aspect of the invention to provide a light-activated opsin polypeptide of the invention that is non-toxic, or substantially non-toxic in cells in which it is expressed. In the absence of light, a light-activated opsin polypeptide of the invention does not significantly alter cell health or ongoing electrical activity in the cell in which it is expressed. In some embodiments of the invention, a light-activated opsin polypeptide of the invention is genetically introduced into a cellular membrane, and reagents and methods are provided herein for genetically targeted expression of light-activated opsin polypeptides. Genetic targeting can be used to deliver a light-activated opsin polypeptide to specific cell types, to specific cell subtypes, to specific spatial regions within an organism, and to sub-cellular regions within a cell, including, cell types such as hippocampal cells, amygdala cells, etc. Routine genetic procedures can also be used to control parameters of expression, such as but not limited to: the amount of a light-activated opsin polypeptide expressed, the timing of the expression, etc.

In some embodiments of the invention a composition for genetically targeted expression of a light-activated opsin polypeptide comprises a vector comprising a gene or functional variat thereof that encodes an opsin polypeptide. As used herein, the term “vector” refers to a nucleic acid molecule capable of transporting between different genetic environments another nucleic acid to which it has been operatively linked. The term “vector” also refers to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors”. Other useful vectors, include, but are not limited to viruses such as lentiviruses, retroviruses, adenoviruses, and phages. Vectors useful in some methods of the invention can genetically insert an opsin polypeptide into dividing and non-dividing cells and can insert an opsin polypeptide into a cell that is an in vivo, in vitro, or ex vivo cell.

Vectors useful in methods of the invention may include additional sequences including, but not limited to, one or more signal sequences and/or promoter sequences, or a combination thereof. In certain embodiments of the invention, a vector may be a lentivirus, adenovirus, adeno-associated virus, or other vector that comprises a gene encoding an opsin polypeptide. An adeno-associated virus (AAV) such as AAV8, AAV1, AAV2, AAV4, AAV5, AAV9, is a non-limiting example of a vector that may be used to express a fusion protein of the invention in a cell and/or subject. Expression vectors and methods of their preparation and use are well known in the art. Non-limiting examples of suitable expression vectors and methods for their use are provided herein.

Promoters that may be used in methods and vectors of the invention include, but are not limited to, cell-specific promoters or general promoters. A non-limiting examples promoters that can be used in vectors of the invention are: ubiquitous promoters, such as, but not limited to: CMV, CAG, CBA, and EF1a promoters; and tissue-specific promoters, such as but not limited to: Synapsin, CamKIIa, GFAP, RPE, ALB, TBG, MBP, MCK, TNT, and aMHC promoters. Methods to select and use ubiquitous promoters and tissue-specific promoters are well known in the art. A non-limiting example of a tissue-specific promoter that can be used to express a light-activated opsin polypeptide in a cell such as a neuron is a synapsin promoter, which can be used to express an opsin polypeptide in embodiments of methods of the invention. Additional tissue-specific promoters and general promoters are well known in the art and, in addition to those provided herein, may be suitable for use in compositions and methods of the invention.

### Stimulation

Certain embodiments of treatment methods of the invention include stimulating an stimulus-activated opsin that has been expressed in a cell in a subject. Stimulation of a targeted opsin with a suitable stimulation means to activate the opsin in the cell. Methods of stimulating opsin polypeptides are well known in the art and may include contacting a cell that expresses an opsin with a light under suitable conditions to activate the opsin.

Methods and apparatus for contacting an expressed opsin with a suitable wavelength of light to activate an opsin ion channel polypeptide or ion pump polypeptide are known in the art. It will be understood that a light of appropriate wavelength for activation will have a power and intensity appropriate for activation of that opsin. It is known in the art that light pulse duration, intensity, and power are some of the parameters that can be altered when activating a light-activated ion channel or ion pump with light. Thus, one skilled in the art will be able to adjust power, intensity, timing, interval of stimulation, etc. appropriately when using a wavelength suitable to activate a selected opsin expressed in a method of the invention. Illumination variables can be altered or “tuned” to optimize activity of a stimulus-activated opsin polypeptide when expressed in a subject and used in a treatment method of the invention. Altering illumination variables such as, but not limited to: wavelength, intensity, pulse width, pulse duration, pulse intervals, overall illumination duration, etc. can be used in conjunction with methods of the invention to optimize treatment for a particular subject, for example to increase activity or decrease activity of the expressed opsin polypeptide. Methods of adjusting illumination variables are well-known in the art and representative methods can be found in publications such as: Lin, J., et al., Biophys. J. 2009 Mar. 4; 96(5):1803-14; Wang, H., et al., 2007 Proc Natl Acad Sci USA. 2007 May 8; 104(19):8143-8. Epub 2007 May 1, each of which is incorporated herein by reference.

It is possible to utilize a narrow range of one or more illumination characteristics to activate a light-activated opsin polypeptide expressed in a subject in a treatment method of the invention. This may be useful to illuminate a light-activated ion channel or ion pump polypeptide that is co-expressed with one or more other light-activated opsins (e.g., channels, pumps, etc.) that can be illuminated with a different set of illumination parameters (for example, though not intended to be limiting, different wavelengths) for their activation, thus permitting controlled activation of a mixed population of light-activated channels and/or pumps. In certain aspects of the invention, methods of treatment include expression of one type of opsin in a subject, and other aspects of the invention include expression of two or more different opsins in a subject. As a non-limiting example, an embodiment of a method of the invention may include expressing in a subject a light-activated ion channel polypeptide that is activated strongly by contact with only blue light and also expressing in the subject a light-activated ion channel or light-activated ion pump that is activated using a different wavelength of light, and that may, in certain embodiments not be activated by blue light. The expression of more than one light-activated opsin in a subject may be used in embodiments of treatment methods of the invention that include expressing one light-activated ion channel (or pump) polypeptide in a certain population of cells in a subject and expressing another light-activated ion channel (or pump) in a separate population of cells, and performing one or more of: contacting cells with different wavelengths of light to activate, activating the different opsins at different time, activating the different opsins for different lengths of time, etc., the opsins and either excite or inhibit activity of the two populations of cells.

Examples of types of cells in which a fusion protein comprising a stimulus-activated opsin polypeptide can be delivered in embodiments of methods of the invention include but are not limited to: cells in a tissue, cell in a subject, cells in an organ, cell in a neural network, cells in a neural pathway, a cell in a brain, etc.

### Methods of Using Opsin Compositions

Opsin polypeptides are well suited for modulating activity of one or more cells in neural pathways in methods of the invention for altering behaviors and/or for treating a metal disorder or a condition. In some embodiments of the invention, a fusion protein comprising a light-activated opsin polypeptide can be expressed in a cell-specific, localized manner. For example, in certain aspects of the invention, an opsin polypeptide is expressed in a hippocampal cell, a dentate gyrus cell, a basal lateral amygdala cell, a parvocellular pyramidal neuron, a magnocellular pyramidal neuron, a Ppplrlb⁺-expressing cell, Rspo2⁺-expressing cell, etc.

Certain embodiments of methods of the invention include expressing a light-activated ion channel polypeptide in a first cell, contacting the expressed light-activated ion channel polypeptide with a light suitable to activate the first cell, wherein the resulting activation of the first cell either directly or indirectly activates at least one additional cell. A non-limiting example of direct activation of one cell by another is a dentate gyrus (DC) cell that projects to a basal lateral amygdala (BLA) cell and activation of the DC cell using a method of the invention results in activation of the BLA cell. Thus, certain embodiments of methods of the invention can comprise activating one cell wherein that activation directly activates a second cell. In certain of such embodiments, a downstream cell does not express the opsin expressed in the upstream cell. In some aspects of the invention a downstream cell does express the opsin expressed in the upstream cell. In some aspects of the invention a downstream cell may express one or more of: the same opsin and a different opsin than the opsin expressed in the upstream cell. A non-limiting example of indirect activation of one cell by another is a DC cell that projects to a BLA cell, which projects to a nucleus accumbens (NAcc) cell, wherein activation of the DC cell using a method of the invention results in activation of the BLA cell, and also activation of the NAcc cell. Thus, certain embodiments of methods of the invention can comprise activating one cell wherein that activation indirectly activates a second cell.

It has now been identified that stimulating a cell in the DG of a subject can result in stimulation of a second cell that is located in the BLA. As used herein, the DG cell is referred to as being “upstream” in relation to the BLA cell, meaning that stimulation of the DG cell results in activation of the BLA cell. A first cell may be directly or indirectly upstream in relation to a second cell. For example, a DG cell can be considered to be directly upstream of the BLA cell and indirectly upstream of the NAcc cell.

It has now been identified that reactivation of positive memory engrams by stimulating in the DC activates the hippocampus→amygdala→nucleus accumbens (NAcc) projection. Thus, a method of the invention may comprise expressing a light-activated ion channel polypeptide in one or more cells in the hippocampus (for example DG cells) contacting the cells with a suitable light to activate the opsin, thereby activating a cell in the BLA to which the activated hippocampal cell (which in some aspects of the invention may be a DG cell) projects. In addition, methods of the invention, in some aspects, include activation of specific cells in the amygdala, for example, activating a Ppplrlb⁺-expressing cell and/or activating an Rspo2⁺-expressing cell.

Methods of the invention can be used to express one or more light-activated opsins in a specific cell type permitting modulation of electrical activity of that cell, which permits control of activity of that cell, and in some embodiments of the invention, modulating an activity of a downstream cell, using suitable illumination. It will be understood that the type and level of modulation of electrical activity and ion flux in a cell will depend, in part, on the light-activated opsin that is expressed in the cell as part of the fusion protein used in methods of the invention. Art-known methods can be used to select suitable stimulation parameters such as type of stimulation, illumination wavelength, intensity, pulse rate, etc. for use with compositions and methods of the invention expressed in cells and membranes. See for example: U.S. Pat. No. 8,957,028; U.S. Pat. No. 9,309,296; U.S. Pat. No. 9,284,353; U.S. Pat. No. 9,249,234; U.S. Pat. No. 9,101,690; PCT Pub. No. WO2013/07123; US Pat Pub No. 20120214188; US Pat Pub. No. 20160039902; US Pat Pub No. 20140223679; Packer, A. M. et al., 2012 Nature Methods December 9(12):1202-1205; and Oron, D. et al., Progress in Brain Research, Chapter 7, Volume 196, 2012, Pages 119-143: the content of each of which is incorporated by references in its entirety herein.

Certain aspects of the invention include methods for modulating one or more characteristics of a cell, such as, but not limited to: electrical activity in a cell and ion flux across a cell membrane. Compositions and methods of the invention can be used in a cell and/or a subject as a means with which to: modulate ion flux across a membrane of a cell, treat a disorders and/or conditions in the cell or subject, identify a candidate agent that when contacted with a cell expressing a fusion protein of the invention modulates electrical activity in the cell, identify a candidate agent that when contacted with a cell expressing a fusion protein of the invention modulates ion flux across a membrane of the cell, etc. In some aspects of the invention, methods and apparatuses that are described herein can be used to image and detect an effect of activating an opsin polypeptide that is expressed in a cell as part of a fusion protein of the invention. Numerous methods for expressing one or more light-activated opsin polypeptides in a host cell and/or a host subject are known in the art and the compositions and methods of the invention may be used in conjunction with such methods to enhance selective activation of a cell in which a fusion protein of the invention is expressed.

Methods and compositions of the invention permit selective expression of a light-activated ion channel polypeptide or a light-activated ion pump polypeptide in a predetermined cell type in a subject, followed by activation or inhibition of that cell using illumination. Methods and compositions of the invention provide an efficient and selective means to localize light-activated opsin polypeptides in specific cell types and then to activate the expressed opsin polypeptide to modulate and control activity of the cell in which the opsin is expressed and also activity in downstream cells.

Working operation of a prototype of this invention has been demonstrated in vivo, by genetically expressing a fusion protein comprising an opsin polypeptides in specific cells in subjects, illuminating the cells with suitable wavelengths of light to activate the opsin, and demonstrating behavioral changes in the subject.

### Cells and Subjects

A cell used in methods and with compositions of embodiments of the invention may be an excitable cell. In certain aspects of the invention, a light-activated opsin polypeptide is expressed in one or more cells in a subject. As used herein, the term “plurality” of cells means two or more cells. A non-limiting example of a cell in which a fusion protein comprising a light-activated opsin polypeptide may be expressed in a treatment method of the invention is a vertebrate cell, which in some embodiments of the invention may be a mammalian cell. Examples of cells in which a fusion protein comprising an opsin polypeptide can be expressed, and/or cells that can be activated by a cell in which an opsin polypeptide is expressed are excitable cells, which include cells able to produce and respond to electrical signals. Examples of excitable cell types include, but are not limited to neurons. A cell in which a fusion protein of the invention is expressed may be a single cell, an isolated cell, a cell that is one of a plurality of cells, a cell that is one in projection or circuit of two or more directly or indirectly connected cells, a cell that is one of two or more cells that are in physical contact with each other, etc.

Non-limiting examples of cells that may be used in methods of the invention, or to which methods of the invention may be applied, include cells that are one or more of: nervous system cells, neurons, hippocampal cells, amygdala cells, basal lateral amygdala cells, dentate gyrus cells, Ppplrlb⁺-expressing cells, and Rspo2⁺-expressing cells. In some embodiments, a cell used in conjunction with the invention may be a cell that is in a subject not known to have, or suspected of having a mental disorder or abnormal condition. In some embodiments, a cell used in conjunction with methods and compositions of the invention may be a cell in a subject diagnosed as having a mental disorder or condition to be treated. Non-limiting examples of cells to which treatment methods of the invention may be applied are: a DG cell in a subject with a mental disorder, a BLA cell in a subject with a mental disorder, a hippocampal cell in a subject with a mental disorder, etc. In some embodiments of the invention, a cell may be a control cell. In some aspects of the invention a cell can be a model cell for a disorder, disease or condition.

In certain embodiments of treatment methods of the invention, a fusion protein comprising a light-activated opsin polypeptide may be expressed in one or more cells in a subject (in vivo cells). Light-activated opsin polypeptides expressed in fusion proteins may be delivered to and expressed in and activated in living subjects, etc. As used herein, the term “subject” may refer to a: human, non-human primate, cow, horse, pig, sheep, goat, dog, cat, rodent, or other host organism. As used herein the term “host” means the subject or cell in which a fusion protein is expressed as part of an embodiment of a treatment method of the invention. In some aspects of the invention a host is a vertebrate subject. In certain embodiments of the invention, a host is a mammal. In certain aspects of the invention a host is a human.

### Controls and Candidate Compound Testing

Using certain embodiments of compositions and methods of the invention, one or more light-activated opsin polypeptides can be expressed in a localized region of subject, for example, in the DG or BLA, and methods to stimulate and determine a response in the cell or in the subject to activation of the light-activated opsin polypeptide can be utilized to assess changes in cells, tissues, and subjects in which they are expressed. Some embodiments of the invention include directed delivery of light-activated opsins to a cell in a subject to identify effects of one or more candidate compounds on the cell, tissue, and/or subject in which the light-activated opsin is expressed. Results of testing one or more activities of a light-activated opsin polypeptide of the invention can be advantageously compared to a control.

As used herein a control may be a predetermined value, which can take a variety of forms. It can be a single cut-off value, such as a median or mean. It can be established based upon comparative groups, such as cells or tissues that include the light-activated opsin polypeptide of the invention and are contacted with light, but are not contacted with the candidate compound and the same type of cells or tissues that under the same testing condition are contacted with the candidate compound. Another example of comparative groups may include cells or tissues that have a disorder or condition and groups without the disorder or condition. Another comparative group may be cells from a group with a family history of a disease or condition and cells from a group without such a family history. A predetermined value can be arranged, for example, where a tested population is divided equally (or unequally) into groups based on results of testing. Those skilled in the art are able to select appropriate control groups and values for use in comparative methods of the invention.

### Methods of Treating

Some aspects of the invention include methods of treating a disorder or condition in a subject by expressing a fusion protein comprising an opsin in a cell of a subject, and activing the expressed opsin under suitable parameters to treat the disorder or condition. Treatment methods of the invention may include administering to a subject in need of such treatment, a therapeutically effective amount of a vector encoding a fusion protein comprising a light-activated opsin polypeptide to treat the disorder. In certain aspects of the invention, a therapeutically effective amount of a cell that comprises a fusion protein may be administered to a subject in a treatment method of the invention. It will be understood that a treatment may be a prophylactic treatment or may be a treatment administered following the diagnosis of a disorder or condition. A treatment of the invention may reduce or eliminate a symptom or characteristic of a disorder or condition or may eliminate the disorder or condition. It will be understood that a treatment of the invention may reduce or eliminate progression of a disorder or condition and may in some instances result in the regression of the disorder or condition. A treatment need not entirely eliminate the disorder or condition to be effective.

In certain aspects of the invention, a means of expressing in a cell of a subject, a fusion protein comprising an opsin polypeptide may comprise: administering to a cell in a subject a vector that encodes a fusion protein comprising the opsin polypeptide; administering to a subject a cell in which a fusion protein comprising the opsin polypeptide is present; or administering a fusion protein comprising the opsin polypeptide to a subject. Delivery or administration of a fusion protein for use in a method of the invention may include administration of a pharmaceutical composition that comprises a cell, wherein the cell expresses the opsin polypeptide. Administration of an opsin polypeptide, may, in some aspects of the invention include administration of a pharmaceutical composition comprising a vector, wherein the vector comprises a nucleic acid sequence encoding an opsin polypeptide, wherein the administration of the vector results in expression of a fusion protein comprising the opsin polypeptide in one or more cells in the subject. In some aspects of the invention, targeted expression of an opsin polypeptide in a particular cell type in a subject may be part of a treatment method. It will be understood that in some aspects of the invention, the starting level of expression of a particular opsin in a cell in a subject may be zero and a treatment method of the invention may be used to increase that level above zero. In certain aspects of the invention, for example in a subsequent delivery of a fusion protein comprising the opsin polypeptide to a subject, a level of expression of the opsin may be greater than zero, with one or a plurality of the opsin polypeptides present the subject, and a treatment method of the invention may be used to increase the expression level of the opsin polypeptide in the subject. As used herein, the terms: “administer” and “deliver” in the context of a treatment method of the invention include any means suitable to result in expression of a stimulus-activated opsin in a cell in a subject. Delivery or administration may include means such as, but not limited to: vector delivery to the subject, fusion protein delivery to the subject, cell delivery to the subject, etc.

An effective amount of a stimulus-activated opsin polypeptide in a treatment method of the invention is an amount that results in expression of the opsin in subject at a level or amount that is beneficial for the subject. An effective amount may also be determined by assessing physiological effects of administration on a subject such as a decrease in symptoms of a disorder or condition to be treated, following administration. Other behavioral and functional assessments will be known to a skilled artisan and can be employed for measuring a level of a response to a treatment of the invention. The amount of a treatment may be varied for example by increasing or decreasing the amount of the opsin polypeptide administered, by changing the therapeutic composition in which the opsin polypeptide is administered, by changing the route of administration, by changing the dosage timing, by changing expression conditions of a fusion protein, by changing the stimulation parameters (wavelength, frequency, interval of stimulation, length of stimulation, etc.) of the expressed opsin polypeptide, and so on.

An effective amount will vary with the particular condition being treated, the age and physical condition of the subject being treated; the severity of the condition, the duration of the treatment, the nature of the concurrent therapy (if any), the specific route of administration, and the like factors within the knowledge and expertise of a health practitioner. For example, an effective amount may depend upon the location and number of cells in the subject in which the opsin polypeptide is to be expressed. An effective amount may also depend on the location of the tissue to be treated. Factors useful to determine an effective amount of a therapeutic compound or composition are well known to those of ordinary skill in the art and can be addressed with no more than routine experimentation. It is generally preferred that a maximum dose of a composition to express and stimulate an opsin polypeptide, and/or to alter the length or timing of stimulation of the expressed opsin polypeptide in a subject (alone or in combination with other therapeutic agents) be used, that is, the highest safe dose or amount according to sound medical judgment. It will be understood by those of ordinary skill in the art, however, that a patient, also referred to herein as a subject, may insist upon a lower dose or tolerable dose for medical reasons, psychological reasons or for virtually any other reasons.

An opsin polypeptide for use in methods of the invention may be administered using art-known methods. The manner and dosage administered may be adjusted by the individual physician, healthcare practitioner, or veterinarian, particularly in the event of any complication. The absolute amount administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutical compositions that include a fusion protein comprising an opsin polypeptide (or an encoding polynucleotide) for treatment methods of the invention may be administered to a subject: singly (alone), in combination with each other, and/or in combination with other drug therapies or other treatment regimens that are administered to the subject. A pharmaceutical composition used in the foregoing methods may contain an effective amount of a therapeutic compound (a stimulus-activated opsin polypeptide) that will increase the level of the opsin polypeptide to a level that when contacted with a suitable stimulus (e.g. illumination) parameters that will produces the desired response in a unit of weight or volume suitable for administration to a subject. In some embodiments of the invention, a pharmaceutical composition of the invention may include a pharmaceutically acceptable carrier.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. In certain embodiments of the invention, such preparations may contain salt, buffering agents, preservatives, compatible carriers, aqueous solutions, water, etc. When used in medicine, the salts may be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

One or more of an opsin polypeptide or encoding polynucleotide thereof, or a cell or vector comprising a nucleic acid sequence encoding an opsin polypeptide, may be administered, for example in a pharmaceutical composition, directly to a cell or tissue in a subject. Direct tissue administration may be achieved by direct injection, and such administration may be done once, or alternatively a plurality of times. If administered two or more times, the polypeptides, polynucleotides, cells, and/or vectors may be administered via different routes. As a non-limiting example, a first (or the first few) administrations may be made directly into an affected tissue while later administrations may be into a different tissue.

A dose of a pharmaceutical composition of the invention that is administered to a subject to increase the level of a desired stimulus-activated opsin polypeptide in one or more cells of the subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The amount and timing of activation (also referred to herein as “stimulation”) of an opsin polypeptide delivered in a method of the invention (e.g., light wavelength, pulse length, length of light contact, duration of activation, intensity of light, etc.) that has been administered to a subject can also be adjusted based on efficacy of the treatment in a particular subject. Parameters for illumination and activation of an opsin delivered to a subject using a method of the invention, can be determined using teaching herein in conjunction with art-known methods, without requiring undue experimentation.

In some aspects of the invention, a treatment is an “acute” treatment and in certain aspects of the invention a treatment is a “chronic” treatment. A chronic treatment may comprise treatment over a longer time versus that of a single acute treatment. In some aspects of the invention, a chronic treatment may be a single treatment and a chronic treatment an ongoing treatment that may include two or more periods of illumination, etc. Parameters of a chronic treatment may, in some aspects of the invention, include one or a combination of: one or more administrations of a light-activated opsin polypeptide to a subject, one or more contacts with a suitable activating light, an extended period of time of contact with a suitable activating light, or other sustained treatment of the subject.

Various modes of administration known to the skilled artisan can be used to effectively deliver a pharmaceutical composition to increase the level of an opsin polypeptide in a desired cell in a tissue or body region of a subject. Methods for administering such a composition or pharmaceutical composition of the invention may be intravenous, intracavity, intrathecal, intrasynovial, intravitreal, trans-tissue, or other suitable means of administration. The invention is not limited by the particular modes of administration disclosed herein. Standard references in the art (e.g., Remington, The Science and Practice of Pharmacy, 2012, Editor: Allen, Loyd V., Jr, 22^{nd} Edition) provide modes of administration and formulations for delivery of various pharmaceutical preparations and formulations in pharmaceutical carriers. Numerous means for administrating of opsins to subjects and suitable parameters and methods for stimulating such opsins, are known and available in the art.

Other protocols which are useful for the administration of a therapeutic compound of the invention will be known to a skilled artisan, in which the dose amount, schedule of administration, sites of administration, mode of administration (e.g., intra-organ) and the like vary from those presented herein. Methods of delivering light-activated opsin molecules in vectors, and methods of expressing fusion proteins that include light-activated opsin molecules that are suitable for use in methods of the invention, include those described herein and other methods known in the art.

Administration of a cell or vector to increase expression of an opsin polypeptide in one or more cells in a mammal other than a human; and methods to treat disorders or conditions, e.g. for testing purposes or veterinary therapeutic purposes, may be carried out under substantially the same conditions as described above. It will be understood that embodiments of the invention are applicable to both human and animals. Thus this invention is intended to be used in husbandry and veterinary medicine as well as in human therapeutics.

### Disorders, Diseases and Conditions

Methods of the invention may be used to express light-activated opsin polypeptides to cells in subjects, and to activate the expressed opsins in a manner that alters voltage-associated cell activities. Such methods may be used to treat disorders such as psychiatric disorders including, but not limited to: depression, post-traumatic stress disorder (PTSD) in a subject. Behavioral or psychiatric characteristics that may be treated using methods of the invention include, but are not limited to: mood disorders, anxiety, hyper-vigilance, social anxiety, and other aspects associated with depression and/or PTSD.

In some aspects of the invention, compositions and methods of the invention, the term “treat” encompasses “augmenting” a condition that may not be considered to be a pathological condition. For example, a treatment method of the invention may be used in a subject who does not have a disorder such as PTSD or depression but rather with a desired goal of treatment of enhancing a behavior in the subject that is not considered to be indicative of a disorder. For example, methods of the invention may be used to elevate mood, elevate activity level, increase social interaction, increase an attentive state, increase an arousal state, or alter other behavioral characteristics from a level considered to near, at, or above a level considered “normal” and non-pathological. Thus, certain aspects of methods of the invention can be used to enhance and improve certain behavioral and activity characteristics in subjects diagnosed with or suspected of having a mental or psychiatric disorder. A level that is considered “normal”, or above normal, and are not considered to be pathological, and/or to be associated with a pathology.

In some embodiments of the invention, a fusion protein comprising a light-activated opsin polypeptide is administered to a subject who has, or is suspected of having depression and the opsin polypeptide is activated in a suitable manner to reactivate a positive memory engram in the subject as a treatment of the depression. In some embodiments of the invention, a fusion protein comprising a light-activated opsin polypeptide is administered to a subject who has, or is suspected of having PTSD and the opsin polypeptide is activated in a suitable manner to reactivate a positive memory engram in the subject as a treatment of the PTSD.

Additional methods of the invention include methods of conditioning behaviors (positive or negative) in a subject with a neutral environmental context. In some embodiments of methods of the invention, a positive behavior is conditioned in a subject to a neutral environmental context. In this method, a subject is exposed to a neutral environmental stimulus, while a Ppplrlb⁺-expressing cell in the BLA of the subject is simultaneously activated. Embodiments of these methods may include: expressing in one or more cells in a subject a stimulus-activated opsin polypeptide, wherein the cell in which the stimulus-activated opsin polypeptide expressed is a Ppplrlb⁺-expressing cell or is a cell that when activated, activates a Ppplrlb⁺-expressing cell in the subject. Thus, in certain embodiments of the invention, a Ppplrlb⁺-expressing cell in the subject can be activated by expressing in the cell a stimulus-activated opsin polypeptide and contacting the expressed stimulus-activated opsin polypeptide with a suitable light to activate the Ppplrlb⁺-expressing cell. The invention, in some aspects, includes methods of conditioning a negative behavior in a subject to a neutral environmental context by expressing in one or more cells in a subject a stimulus-activated opsin polypeptide, wherein the stimulus-activated opsin polypeptide is expressed in an Rspo2⁺-expressing cell or is expressed in a cell that when activated, activates an Rspo2⁺-expressing cell in the subject. The expressed stimulus-activated opsin polypeptide is then activated and the subject exposed to a neutral environmental context at a time simultaneous with the activation of the expressed stimulus-activated opsin polypeptide; wherein the simultaneous activation and exposure conditions a negative behavior in the subject to the neutral environmental context.

In some embodiments of the invention, a fusion protein comprising a light-activated opsin polypeptide is administered to a subject and while contacting the expressed opsin polypeptide with suitable light to activate the opsin, exposing the subject who has, or is suspected of having depression, PTSD, another mental disorder, or other condition, and the opsin polypeptide is activated in a suitable manner to reactivate a positive memory engram in the subject as a treatment of the depression. Overall, methods, compounds, and compositions have now been identified that are useful for actions such as, but not limited to: stimulation or inhibition of specific neurons in the BLA, reactivation of memory engrams, and modulation of activity in specific cells of the hippocampus→amygdala→NAcc pathway. Such methods and others encompassed by the invention can be used to treat mental disorders and conditions in subjects.

### Testing Methods

As a non-limiting example, a treatment method of the invention can be used at a time that is one or more of: before, during, or after administration of a candidate therapeutic agent or compound that is tested to see if it augments the treatment of the invention, inhibits efficacy of the treatment of the invention, or is synergistic with a treatment method of the invention. In such methods of the invention, additional and combination treatments for diseases, disorders, or conditions can be assessed and efficacy determined. In one embodiment of the invention, in a subject, a test cell in which a fusion protein comprising a light-activated opsin polypeptide is expressed according to a method of the invention is contacted with a light that depolarizes the cell or otherwise alters ion flux across the cell membrane and the subject is also administered a candidate compound. The cell and/or subject that include the cell can be monitored for the presence or absence of a change that occurs in test conditions versus a control condition. For example, in a cell, an activity modulation in the test cell may be a change in the depolarization/hyperpolarization of the test cell, a change in the subject's mood, activity level, anxiety level, behavior, or other characteristic of a disease, disorder, or condition being treated with the method of the invention. Art-known methods can be used to assess electrical activity and ion flux activity and changes in mood, affect, activity levels, etc. with or without additional contact with a candidate compound.

The present invention in some aspects includes preparing nucleic acid sequences and polynucleotide sequences; expressing in cells and membranes polypeptides encoded by the prepared nucleic acid and polynucleotide sequences; illuminating the cells and/or membranes with suitable light, and which results in modulation of electrical activity and or ion flux in the cells and across membranes. The ability to controllably alter one or more of: voltage across membranes; ion flux across members, cell depolarization, cell hyperpolarization using contact of the expressed opsin polypeptide with light has been demonstrated for numerous opsins that can be included in compositions and methods of the invention. The present invention enables targeted expression and localization of opsins in cells that are involved in memory engrams, and cells that can be modulated in treatments for mental disorders such as depression, PTSD, and conditions such as fear, anxiety, and social inhibition. Compositions and treatment methods of the invention and their use have broad-ranging applications for treatment of mental disorders, augmenting non-pathogenic behaviors, and research applications, some of which are describe herein.

### Kits

The invention, in part also includes kits that can be used in treatment methods of the invention. Such kits may comprise one or more of: a polynucleotide that encodes stimulus-activated opsin polypeptide, a composition of the invention, a compound of the invention, vectors, components to include in vectors, cell, etc. A kit may also include instructions for delivering a treatment method of the invention to a subject.

### EXAMPLES

### Example 1

### Materials/Methods

### Subjects.

The c-fos-tTA mice were generated by crossing TetTag (31) mice with C57BL/6J mice and selecting for those carrying the c-fos-tTA transgene. Littermates were housed together before surgery and received food and water ad libitum. All mice were raised on a diet containing 40 mg kg⁻¹ doxycycline (Dox) for a minimum of 1 week before receiving surgery at age 12-16 weeks. Post-operation, mice were individually housed in a quiet home cage with a reverse 12 h lightdark cycle, given food and water ad libitum, and allowed to recover for a minimum of 2-3 weeks before experimentation. All animals were taken off Dox for an undisturbed 42 h to open a time window of activity-dependent labelling. In this system, the promoter of c-Fosan immediately early gene often used as a marker of recent neural activity—was engineered to drive the expression of the tetracycline transactivator (tTA), which in its protein form binds to the tetracycline response element (TRE). Subsequently, the activated TRE drives the light responsive channelrhodopsin-2 (ChR2). Importantly, the expression of ChR2 only occurs in the absence of doxycycline (Dox) from the animal's diet, thus permitting inducible expression of ChR2 in correspondingly active cells.

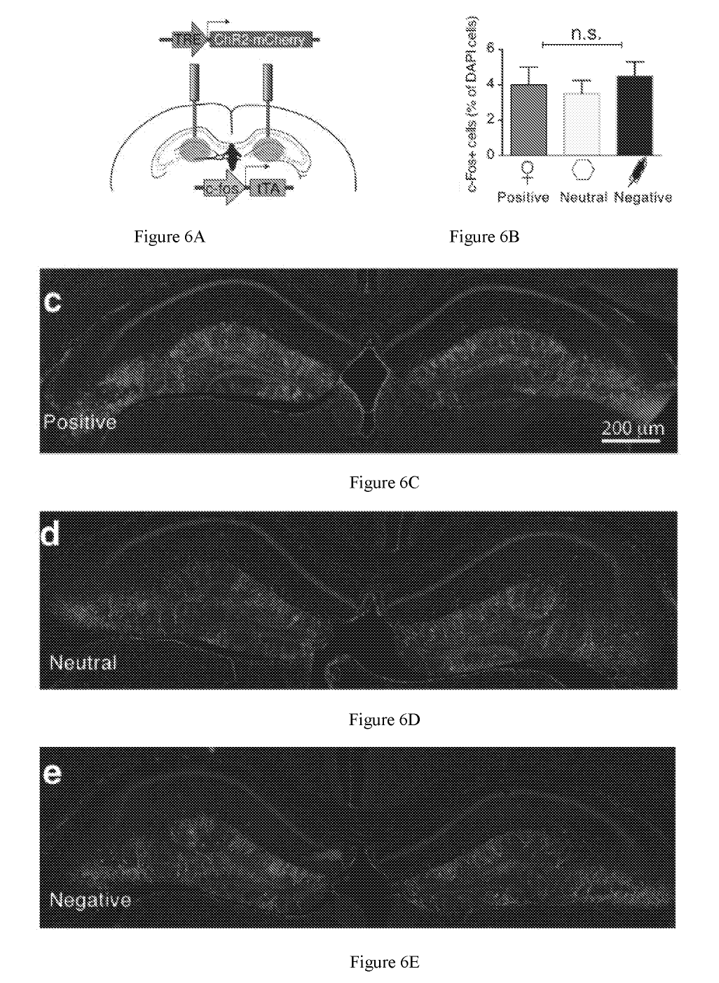

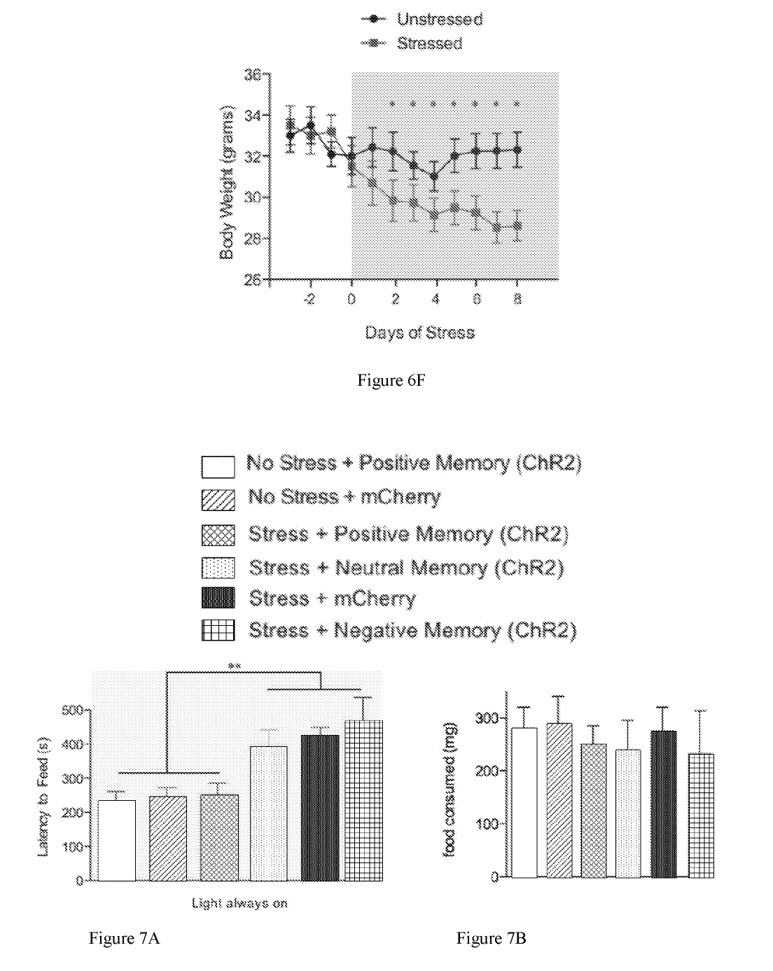

Each group of male mice was exposed to all three subsequent treatments for 2 hours and randomly assigned which experience would occur while off Dox; a negative experience (that is, a single bout of immobilization stress, see below), a naturally rewarding experience [that is, exposure to a female conspecific while in a modified home cage, as previously reported (32)], and a neutral experience (that is, exposure to a conditioning chamber). For female exposure, single-caged male mice were moved to a behavior room distinct from the housing room and with dim lighting conditions. Next, the cage tops were removed and a 4-sided (31×25×30 cm) white box was placed over the home cage, after which a female mouse was introduced to the home cage. Importantly, this modification to the home cage during female exposure ensured similar levels of dentate gyrus labelling as the neutral and negative memory exposure groups (FIG. 6). Each group was taken off Dox only during one of the aforementioned treatments and placed back on Dox immediately afterwards. The subjects were age-matched and split into two groups: a stressed group and a non-stressed group. Non-stressed animals remained in their home cages before experimentation. Stressed animals underwent 2-3 h of chronic immobilization stress (CIS) each day for ten consecutive days before behavioral testing using Mouse DecapiCone disposable restrainers. All procedures relating to mouse care and treatment conformed to the institutional and National Institutes of Health guidelines for the Care and Use of Laboratory Animals. Sample sizes were chosen on the basis of previous studies (32-34); variance was similar between groups for all metrics measured. No statistical methods were used to predetermine sample size.

### Virus Constructs and Packaging.

The pAAV₉-TRE-ChR2-mCherry and pAAV₉-TRE-mCherry plasmids were constructed as previously reported (33). The pAAV₉-TRE-ArchT-eGFP was constructed by replacing the ChR2-eYFP fusion gene in the pAAV₉-TRE-ChR2-eYFP plasmid from Liu et al. (34) with a fusion gene of ArchT-eGFP from Han et al. (35). These plasmids were used to generate AAV₉ viruses by the Gene Therapy Center and Vector Core at the University of Massachusetts Medical School. Viral titrations were 8×10¹² genome copy per ml for AAV₉-TRE-ChR2-mCherry, 1.4×10¹³ genome copy per ml for AAV₉-TRE-mCherry, and 0.75 to 1.5×10¹³ genome copy per ml for AAV9-TRE-ArchT-eGFP.

### Stereotactic Injection, Cannulation, and Fibre Optic Implants.

All surgeries were performed under stereotaxic guidance and subsequent coordinates are given relative to bregma. Animals were anaesthetized using 500 mg kg⁻¹ Avertin before receiving bilateral craniotomies using a 0.5 mm diameter drill bit at 2.2 mm anterioposterior (AP), ±1.3 mm mediolateral (ML) for dentate gyrus injections. All mice were injected with 0.15 μl of AAV9 virus at a controlled rate of 0.6 μl min⁻¹ using a mineral oil-filled glass micropipette joined by a microelectrode holder (MPH6S; WPI) to a 10 μl Hamilton microsyringe (701LT; Hamilton) in a microsyringe pump (UMP3; WPI). The needle was slowly lowered to the target site at 2.0 mm dorsoventral (DV). The micropipette remained at the target site for another 5 minutes post-injection before being slowly withdrawn. A bilateral optical fibre implant (200 μm core diameter; Doric Lenses) was lowered above the injection site (˜1.6 mm DV for dentate gyrus) and three jewellery screws were secured into the skull at the anterior and posterior edges of the surgical site to anchor the implant. For mice used in pharmacological manipulations, bilateral guide cannula (PlasticsOne) were implanted above the NAcc (+1.2 mm AP; ±0.5 mm ML; 3.25 mm DV). Mice used in the BLA-to-NAcc or mPFC-to-NAcc experiments received bilateral injections (0.2 μl to 0.3 μl) of TRE-ArchT-eGFP or TRE-eGFP into the BLA (−1.46 mm AP; ±3.20 mm ML; −4.80 mm DV), NAcc (+1.2 mm AP; ±0.50 mm ML; −4.3 mm DV), or the mPFC (+1.70 mm AP; ±0.35 mm ML; −2.70 mm DV). These mice were then injected with TRE-ChR2-mCherry into the dentate gyrus and received bilateral optic fibre implantation as described above (Doric Lenses), as well as bilateral optic fibre implantation over the NAcc (+1.2 mm AP; ±0.50 mm ML; −3.70 mm DV).

Layers of adhesive cement (C&B Metabond) followed by dental cement (Teets cold cure; A-M Systems) were spread over the surgical site and protective cap to secure the optical fiber implant. The protective cap was made from the top portion of a black polypropylene microcentrifuge tube. Mice received intraperitoneal injections of 1.5 mg kg⁻¹ analgesics and were placed on heating pads throughout the procedure until recovery from anaesthesia. Histological studies were used to verify fibre placements and viral injection sites. Only data from mice with opsin or fluorophore expression restricted to the dentate gyrus, BLA or mPFC were used for histological, behavioral and statistical analyses.

### Pharmacological Infusion of Glutamate or Dopamine Receptor Antagonists.

Glutamate antagonists were bilaterally infused into the NAcc as follows: 0.2 μl per hemisphere of NBQX at a concentration of 22.3 mM to antagonize AMPA (α-amino-3-hydroxy-5-methyl-4-isoxazole propionic acid) receptors and 0.2 μl per hemisphere of AP5 at a concentration of 38.04 mM to antagonize NMDA (N-methyl-D-aspartate) receptors. Dopamine receptor antagonists were bilaterally infused into the NAcc as follows: 0.2 μl SCH23390 at a concentration of 6.16 mM to antagonize D1-like receptors and 0.2 μl raclopride at a concentration of 2.89 mM to antagonize D2-like receptors. A 26-gauge stainless steel double internal cannula (PlasticsOne) was used to bilaterally infuse each drug; the internal cannula was connected with a microsyringe pump by a PE20 tube to control the injection rate at 100 nl min⁻¹. The injection cannula was left connected for 5 min before removal to allow for diffusion. Finally, all behavior was performed 20 min following drug infusion.

### Immunohistochemistry.

Mice were overdosed with 750-1000 mg kg⁻¹ Avertin and perfused transcardially with cold PBS, followed by 4% paraformaldehyde (PFA) in PBS. Extracted brains were kept in 4% PFA at 4° C. overnight, then transferred to PBS. A vibratome was used to recover 50-μm coronal slices in cold PBS. Slices were washed with PBS-T (PBS+0.2% Triton X-100), then incubated with PBS-T+5% normal goat serum at 4° C. for 1 h for blocking. For immunostaining, slices were incubated with one or more primary antibodies (1:1000 dilution) at 4° C. for 24 h (600-401-379 Rockland; A10262, Invitrogen; SC-52, Santa Cruz). Three washes of PBS-T for 10 min each were performed on the slices before 1 h incubation with secondary antibody at 1:200 dilution (A11039, Invitrogen; A21429, Invitrogen). Slices were washed three more times in PBS-T for 10 min each, stained with 4′,6-diamidino-2-phenylindole (DAPI; 1:10,000 dilution) to label cell nuclei and mounted with Vectashield H-1200 onto microscope slides.

### Behavioral Assays.

All behavior assays were conducted during the light cycle of the day (7:00-19:00) on animals 12-16 weeks old. Mice were handled for 3-5 days, 2 min per day, before all behavioral experiments.

### Tail Suspension Test.

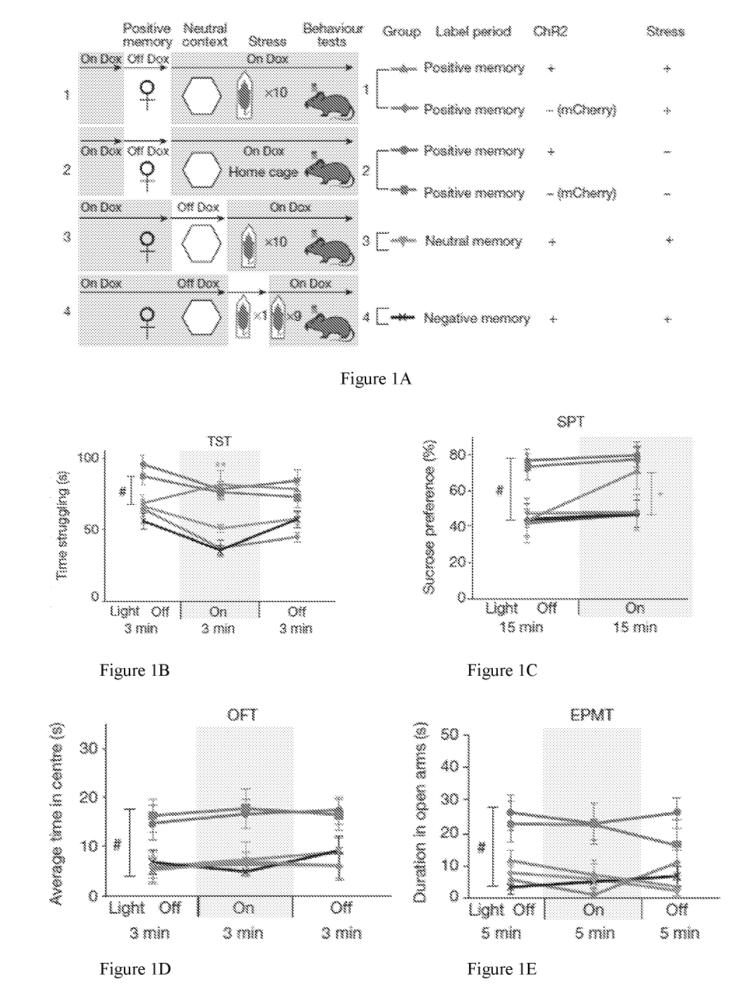

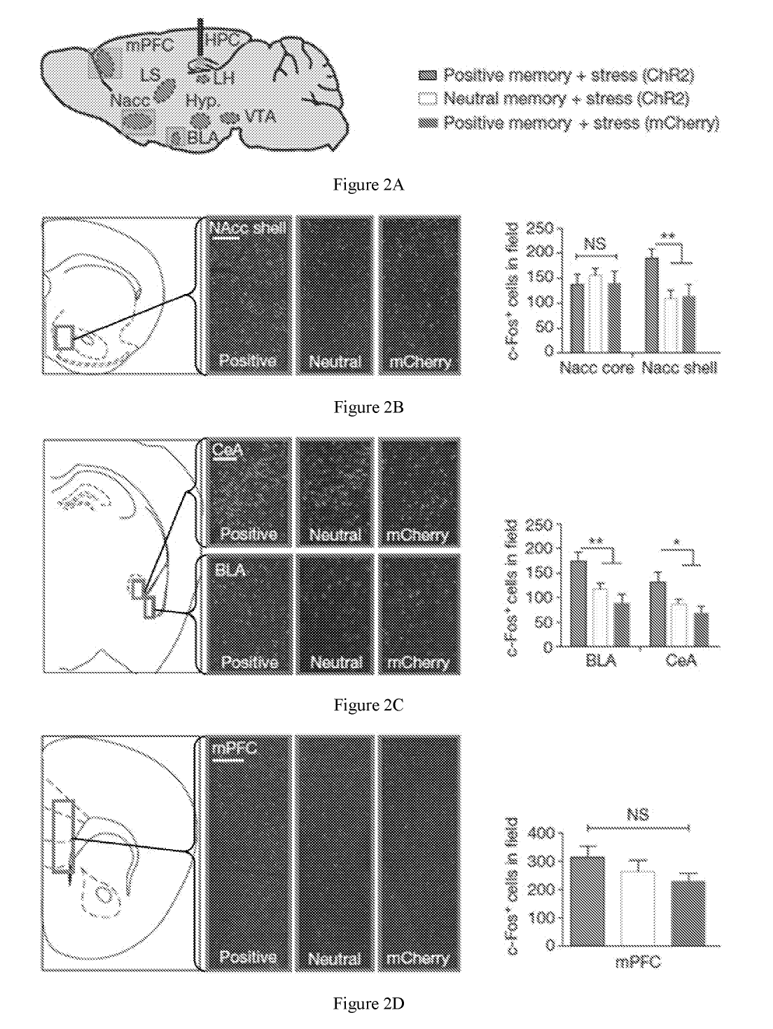

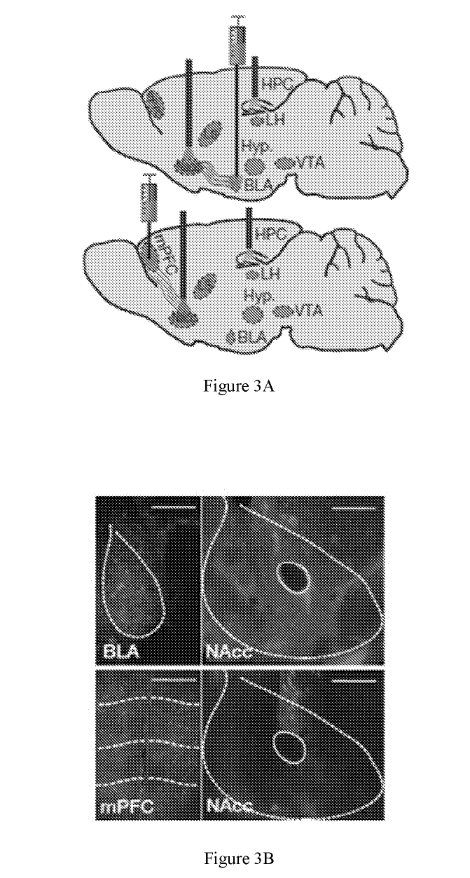

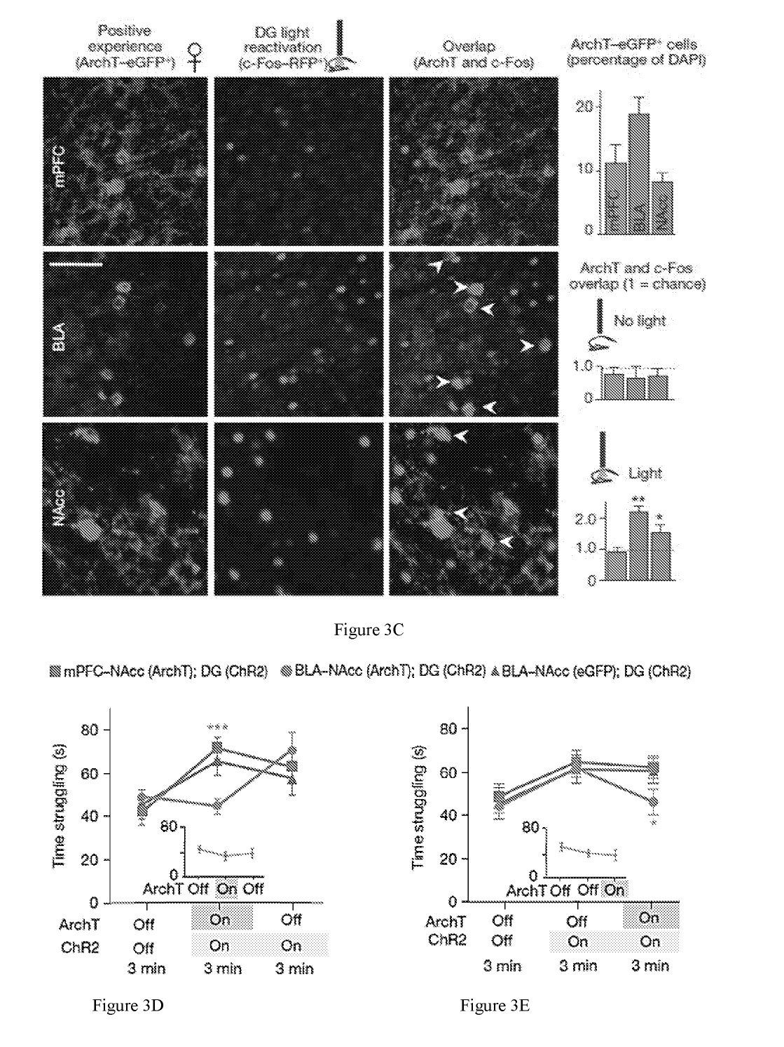

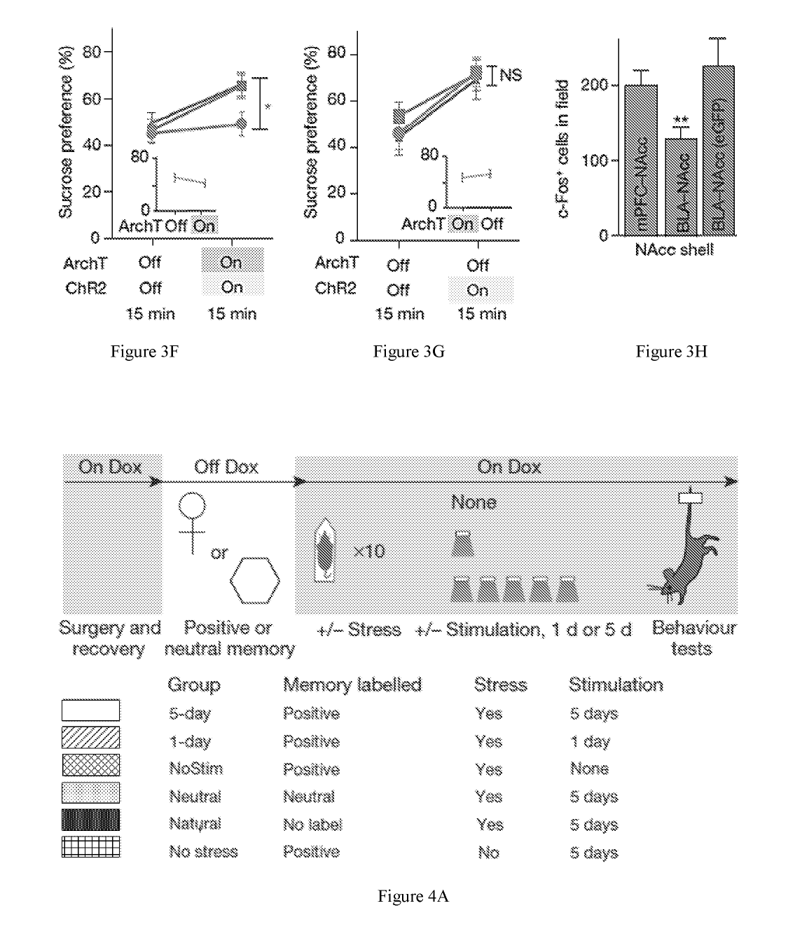

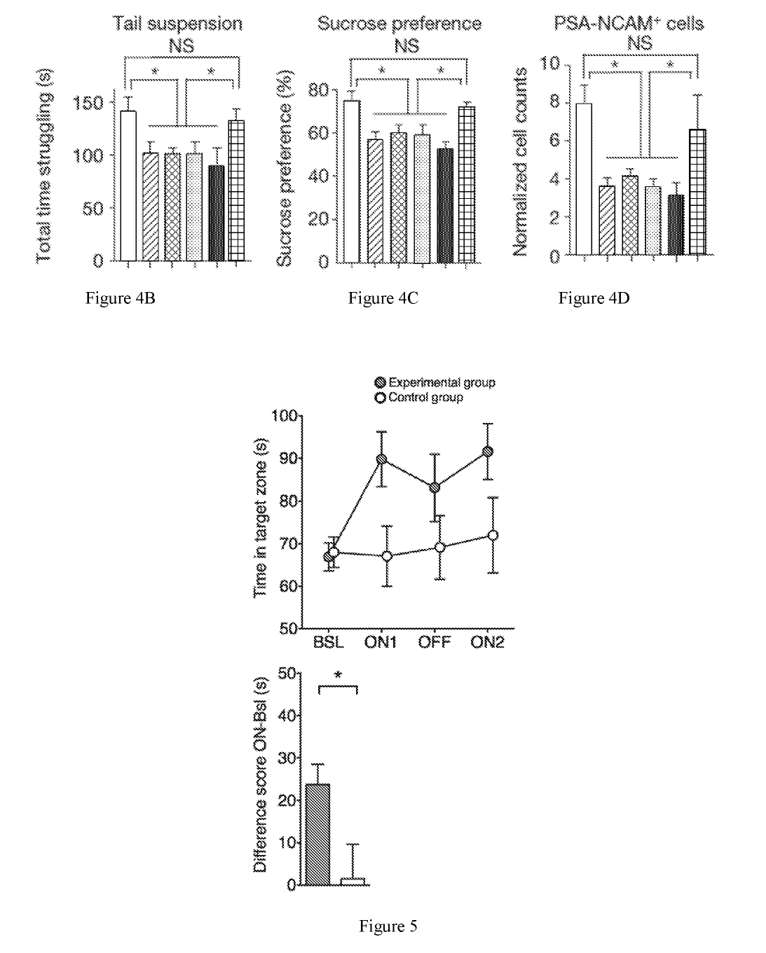

Fibre optic implants on experimental mice were plugged into a patch cord before the tail suspension test. Each subject was hung by its tail from a bar 40 cm from the ground with a single piece of autoclave tape. The animal was positioned such that it had no contact with other objects. Immediately after positioning, video recordings of the animal's movements were taken (Noldus by Ethovision). Blue light stimulation was given at 20 Hz, 15 ms pulse width, ˜15-20 mW. For behavioral data appearing in FIG. 1, all mice were exposed to a 9 min tail suspension test with light stimulation occurring at minutes 3-5, inclusive; for histological data appearing in FIG. 2, all mice were exposed to a 6 min tail suspension test with light stimulation occurring throughout the entire session using the same stimulation parameters described above. For data appearing in FIG. 3, all animals were given a 9 min tail suspension test once a day for 2 days to assess the effects of ArchT inhibition on BLA or mPFC terminals in the NAcc while simultaneously activating ChR2-positive cells in the dentate gyrus. For half of the subjects, on day 1, ArchT-mediated inhibition occurred during minutes 3-5, inclusive, using constant green light at ˜25 mW; dentate gyrus stimulation occurred from minutes 3-8, inclusive. For the other half, ArchT-mediated inhibition occurred during minutes 6-8, inclusive; and dentate gyrus stimulation occurred from minutes 3-8, inclusive. The treatments occurring on days 1 and 2 were counterbalanced within and across groups. A separate cohort of animals were used for the data appearing in the insets of FIG. 3D-G. These groups contained TRE-ChR2-mCherry in the dentate gyrus, as well as bilateral optic fibers over the dentate gyrus, and TRE-ArchT-eGFP in the BLA, as well as optic fibers over the NAcc to inhibit BLA terminals during the appropriate light-on epochs in the TST and SPT. These cohorts, too, were counterbalanced across sessions and only received green light over the NAcc for 3 min during the TST or 15 min during the SPT. For the c-Fos counts appearing in FIG. 3H, all groups underwent a 6 min tail suspension test with blue light delivered to the dentate gyrus and green light delivered to the NAcc throughout the entirety of the session. These groups were sacrificed 1.5 h later for histological analyses. For data appearing in FIG. 4, mice were exposed to a 6 min tail suspension test without light stimulation. An experimenter blind to each mouse condition and light treatment scored all the tail suspension videos by measuring the total time in seconds that each mouse spent struggling throughout the protocol.

### Sucrose Preference Test.

A Med Associates operant chamber—equipped with photolickometers placed on two separate corners of the chamber—was used to count the number of licks made by the mice on lick spouts with direct access to 2% sucrose water solution or water alone. All animals undergoing the sucrose preference protocol were water-restricted for 36 h before each habituation session. These sessions consisted of first plugging the optic fibers on the water-deprived mice to a corresponding patch cord and exposing the mice to the operant chamber, which contained bottles filled only with water. Each exposure occurred on three separate days for 30 min per day. The three habituation sessions occurred interspersed throughout the 10-day chronic immobilization stress protocol (that is, on days 1, 4 and 7 of stress) at least 6 h before or after the stress protocol. In pilot experiments, ˜90% of water-deprived animals failed to sample both photolickometers in the operant chamber even after multiple 30-min habituation sessions (data not shown); to address this issue, a glove box was inserted on its side in the operant chamber such that each subject had a narrow ˜10 cm corridor to explore and find each lick spout. With this modification −90% of animals found both lick spouts during the first and subsequent habituation sessions. Upon completing a habituation session, mice were given water only when 2 h of being placed back into the home cage had elapsed. On the test day (that is, the day on which optical stimulation occurred), the location of each sucrose or water bottle in the chamber was counterbalanced between animal chambers. A 30 min protocol—15 min light off, 15 min light on—was used on all animals. The first 15 min were used to detect the baseline preference; blue light stimulation at 20 Hz, 15 ms pulse width, −15-20 mW, occurred during the second 15 min epoch to detect light-induced changes in preference. For data appearing in FIG. 3, water-deprived animals were exposed to the same 30-min protocol on two separate days. On day 1, after the first 15-min epoch, half of the animals received constant green light stimulation at ˜15 mW [as previously reported (36)] over the NAcc while simultaneously receiving blue light stimulation over the dentate gyrus; the other half received only blue light stimulation over the dentate gyrus. On day 2, the treatments were reversed in a counterbalanced manner. Data was only collected in animals that licked at both spouts in the first 15-min interval; animals that did not discover both lick spouts (as evidenced by licking only one spout during the first 15-min interval) were not given light stimulation, the experiment was terminated early, and the test was repeated the following day. Sucrose preferences were calculated as follows:

$$\frac{total number of licks to sucrose spout}{\begin{matrix}total number of licks to sucrose spout + \\ total number of licks to water spout\end{matrix}} \times 100$$

For the sucrose preference data appearing in FIG. 4, mice were first habituated to two water bottles for 2 days in their home cages. On day 3, two water bottles containing either 2% sucrose or water were placed into the cages in a counterbalanced manner and left undisturbed for 24 h. Sucrose preferences were calculated as follows:

$$\frac{Δ weight of sucrose water}{Δ weight of sucrose water + Δ weight of water} \times 100$$

### Open Field Test.

An open, metal chamber (Accuscan system) with transparent, plastic walls was used for the open field test. Implanted mice were plugged into a patch cord, individually placed into the chamber, and allowed to explore freely for 12 min. An automated video-tracking system (Ethovision by Noldus) was used to track the amount of time spent in the center of the chamber compared to the edges, as well as the total distance travelled across a session. Light stimulation, as described above, was given during minutes 3-5 and 9-11, inclusive.

### Elevated Plus Maze Test.

Implanted animals were plugged into a corresponding patch cord before the beginning of the session and subsequently placed in an elevated plus maze. Two pieces of plastic (30 cm long, 5 cm wide) formed the two arms of the maze that intersected at right angles. One arm was enclosed with plastic black walls, and the other arm was open with no walls. The structure was elevated 60 cm above the floor and mice were placed one at a time at the intersection of the maze facing into an arm with walls to start a trial. Video tracking software (EthoVision by Noldus) was used to track the amount of time the mice spent in the enclosed versus the open arms of the maze throughout a 15-min session. Optical stimulation occurred only during the second 5-min epoch using the same stimulation parameters as noted above.

### Novelty-Suppressed Feeding.

The novelty suppressed feeding paradigm was performed as previously described (37). In brief, food was removed from the subjects' home cages 24 h before testing. The next day, mice were placed for 10 min in an open field apparatus containing bedding with a food pellet at the center on a 1 cm² elevated platform. Light stimulation using the parameters described above occurred throughout the entire session. All behavior was videotaped (Ethovision by Noldus) and latency to feed was scored offline by an experimenter

blind to the experimental conditions for each mouse. Once placed back into their home cages, mice were given a single food pellet, which was weighed before and after a 5-min test to measure for motivation/hunger effects on feeding behavior compared to feeding in a novel environment.

### 5-Day Stimulation Protocol.

For data appearing in FIG. 4, animals were first split into six groups: a group in which dentate gyrus cells previously active during a positive experience were reactivated twice a day for 5 days (5-day group) after the CIS protocol, a group in which such stimulation occurred twice a day for 1 day (1-day group) after the CIS protocol, a group in which no stimulation was delivered (NoStim group) after the CIS protocol, a group in which dentate gyrus cells previously active during a neutral experience were reactivated twice a day for 5 days (Neutral group) after the CIS protocol, a group that did not receive the CIS stress protocol but still had dentate gyrus cells previously active during a positive experience reactivated twice a day for 5 days (NoStress group), and finally, a group that was exposed to a natural social reward (that is, female mouse) twice a day for 5 days (Natural group). Optical stimulation first occurred at 10:00 for 15 min (blue laser, 20 Hz, 15 ms pulse width, ˜15-20 mW) as animals explored an operant chamber, and then again at 15:00 for 15 min using the same conditions. The same behavioral schedule was performed for the Natural group. All groups were exposed for an equal amount of time to each chamber, plugged into a corresponding patch cord, and optical stimulation occurred only in the appropriate groups. Each chamber contained dim lighting, white plastic floors, and no artificial odorants. One day after the final stimulation, all groups were exposed to a 6 min tail suspension or 24 h sucrose preference test as described above.

### Object-Female Association.

Twenty-four wild-type B6 mice were divided in two groups [neutral-object group, that is, control group, and female-object group, that is, experimental group (n=12 per group)]. The learning and testing phases were conducted on the same day, 6 h apart. In the learning phase, all mice spent 30 min in their home cage in the middle of a well-lit room with the lid of the cage and metal grid holding water and food removed and a 30 cm tall white rectangular frame placed around the home cage to prevent mice from escaping. All the boxes contained one target object [counterbalanced objects within and between groups: empty methanol bottle or cryostat liquid bottle (sealed)]. After 3 min exploring the target object, a wild-type female B6 mouse (age 9 to 16 weeks) was introduced in the boxes of the experimental mice and remained there for the next 27 min). The control mice did not experience a female mouse and only experienced the object. After a total of 30 min from the beginning of the learning phase, the object and female mouse were removed and the male mice returned to their holding rooms. In the testing phase, mice were placed in a rectangular arena (70×25×30 cm) with white floors. A video camera resides above the testing chamber where the locations of the subjects were tracked and recorded using Noldus EthoVision XT video tracking software. Two zones (left and right) on either end of the box (30×30 cm) as well as a neutral zone in the center of the box (10 cm) were denoted as part of the arena settings. Mice were introduced in the neutral zone of the empty arena and allowed to explore freely for 3 min. The tracking software monitored which of the two zones each individual mouse preferred. After 3 min, the experimenter introduced two objects [empty methanol bottle or cryostat liquid bottle (sealed)] and placed them in the middle of the left and right zones. For each mouse, one of the objects was the same as the one experienced during training (target object in target zone) and was placed in the least preferred zone. The other object was novel (novel side) and placed in the preferred side. During minutes 6 to 9, objects were absent from the arena. During minutes 9 to 12, the objects were reintroduced in the same positions as minutes 3 to 6.

### Cell Counting.

The number of mCherry or c-Fos immunoreactive neurons in the dentate gyrus and downstream areas were counted to measure the number of active cells during defined behavioural tasks in 3-5 coronal slices (spaced 160 mm from each other) per mouse. Only slices that showed accurate bilateral injections in the dentate gyrus were selected for counting. Fluorescence images were acquired using a microscope with a 320/0.50 NA objective. All animals were sacrificed 90 min post-assay or optical stimulation for immunohistochemical analyses. The number of c-Fos-positive cells in a set region of interest (0.5 mm² per brain area analyzed) were quantified with ImageJ and averaged within each animal. Background autofluorescence was accounted for by applying an equal cutoff threshold to all images by an experimenter blind to experimental conditions. To calculate the percentage of BLA, mPFC, or NAcc cells expressing ArchT-eGFP in FIG. 3c, the number of GFP-positive cells were counted and divided by the total number of DAPI-positive cells in each region. Statistical chance was calculated by multiplying the observed percentage of ArchT-GFP-single-positive cells by the observed percentage of c-Fos-single-positive cells; overlaps over chance were calculated as observed overlap divided by chance overlap:

$$\frac{\frac{( {GFP}^{+} \times c - {Fos}^{+} )}{DAPI}}{chance overlap}$$

A one-way ANOVA followed by Tukey's multiple comparisons or one-sample t-tests were used to analyze data and later graphed using Microsoft Excel with the Statplus plug-in or Prism.

### Neurogenesis.

After all the behavior tests, on the 15th day since the first day of light stimulation, the mice were overdosed with Avertin and perfused transcardially with cold phosphate buffer saline (PBS), followed by 4% paraformaldehyde (PFA) in PBS. Brains were extracted from the skulls and kept in 4% PFA at 4° C. overnight. Coronal slices 50-μm thick were taken using a vibratome and collected in cold PBS. For immunostaining, each slice was placed in PBST (PBS+0.2% Triton X-100) with 5% normal goat serum for 1 h and then incubated with primary antibody at 4° C. for 24 h (1:250 mouse anti-PSA-NCAM, Millipore; 1:500 doublecortin, AB2253, Millipore). Slices then underwent three wash steps for 10 min each in PBST, followed by a 1 h incubation period with secondary antibody (PSA-NCAM: 1:250 AlexaFluor488 anti-mouse, Invitrogen; Doublecortin: 1:300 A21435, Invitrogen). Slices were then incubated for 15 min with 4′,6-diamidino-2-phenylindole (DAPI; 1:10,000) and underwent three more wash steps of 10 min each in PBST, followed by mounting and coverslipping on microscope slides. Images were taken using a Zeiss Axio Imager2 microscope. PSA-NCAM⁺ or doublecortin⁺ cells in the dentate gyrus granule cell layer were counted and normalized to the area of the granule cell layer for each brain slice using ImageJ by a researcher blind to the identities of each animal. After all the data were collected, the identities of each animal were revealed and the data were assigned back into each group for statistical analysis.

### In Vivo Electrophysiology.

As described above, three mice were first bilaterally injected with an AAV₉-TRE-ChR2-mCherry virus into the dentate gyrus followed by lowering a bilateral optic fibre implant into position and cementing it to the skull. Following 10 days for recovery and viral expression, in a separate surgery, mice were chronically implanted with a hyperdrive that housed six independently moveable tetrodes targeting the BLA. To accommodate the optic fibre implant cemented on the skull, the AP coordinate for the hyperdrive was adjusted slightly (centered at AP=−0.85 mm) and implanted at a ˜15° angle. The electrical signal recorded from the tips of the tetrodes was referenced to a common skull screw over the cerebellum and differentially filtered for single unit activity (200 Hz to 8 kHz) and local field potentials (1-200 Hz). The amplified signal from each wire is digitized at 40 kHz and monitored with an Omniplex system (Plexon). Action potentials from single neurons were isolated off-line using time-amplitude window discrimination through Offline Sorter (Plexon). Putative single units were isolated by visualizing combinations of waveform features (square root of the power, peak-valley, valley, peak, principal components, and time-stamps) extracted from wires composing a single tetrode. The average firing rate for isolated neurons was 2.25 Hz±4.14 Hz (mean±s.d.; range 0.01-30.15 Hz). However, the firing rate distribution was highly rightward skewed (median: 0.81 Hz) and more than half of the neurons (62%; 66/106) had firing rates under 1 Hz. After the last recording session, small lesions were made near the tips of each tetrode by passing current (30 μA for ˜10 s) and mice were transcardially perfused and brains extracted for histology using standard procedures.

### Recording and Light Stimulation Protocol.

Each mouse had two recording sessions that occurred on two different days separated by 72 h. Mice were first placed into a small recording chamber. In a single recording session, mice were first bilaterally stimulated in the dentate gyrus with blue light (450 nm; Doric Lenses) for 10 s over 15 such trials in total. As a control, the blue light was replaced with red light (640 nm; Doric Lenses) and the mice were given twelve 10-s trials under this condition. The power output for the blue and red lights emitted from the tips of each patch cord was adjusted to 15-18 mW as measured with a standard photometer (Thor Labs). The blue and red lasers were powered using a laser diode driver (Doric Lenses) triggered by transistor-transistor logic (TTL) pulses emitted from a digital I/O card, and these events were also time-stamped and recorded in the Omniplex system. The recording session lasted ˜20 min and each tetrode was lowered ˜0.25 mm after the first recording session.

### Electrophysiological Data Analysis.

Spiking activity was analyzed using commercial (Neuroexplorer, NEX Technologies) and custom-made software in Matlab (R2014B). To visualize each neuron's trial-averaged activity for the blue and red light stimulation period, a peristimulus time histogram (PSTH) with 100-ms time bins was generated with activity time locked to the onset of the blue or red light, and then smoothed with a Gaussian kernel (θ=127 ms). In order to confirm a response during blue or red light stimulation period, 99% confidence intervals were constructed for the trial-averaged activity using a baseline 2.5 s period of spiking activity before the onset of each light under the assumption of Poisson spiking statistics (for example, Neuroexplorer, NEX Technologies). A neuron was considered to have a response for a particular light stimulation condition if trial averaged activity exceeded the upper (excitatory) or lower (inhibitory) bound of the 99% confidence interval. Neurons were considered activated from dentate gyrus stimulation when a neural response was confirmed for the blue light condition but not the red light condition. For each neuron identified as such, neural activity depicted in the blue and red light PSTH was z-scored, then identified the maximum trial-averaged z-score value from the 2.5 s baseline (Pre) and during blue or red light stimulation (Post). The Pre and Post maximum z-score values for the blue and red light stimulation period was compared using paired t-tests.

### Results/Discussion

Prior studies demonstrate that dentate gyrus cells that express c-Fos during fear or reward conditioning define an active neural population that is sufficient to elicit both aversive and appetitive responses, and that the mnemonic output elicited by these artificially reactivated cells can be updated with new information (6-8). Studies present herein were performed to develop and assess methods to alleviate stress-induced behavioral impairments via a defined set of dentate gyrus cells that are active during a positive experience. Experiments described herein examined how positive episodes interact with psychiatric-disease-related behavioral states, including depression-related impairments.

Experimental methods included labelling and manipulation of memory engram cells (see Methods) (6-8). Animals that were taken off doxycycline were exposed to a naturally rewarding experience (8) (that is, exposure to a female mouse in a modified home cage, hereafter referred to as a ‘positive experience’ and further validated in FIG. 5), a neutral context (hereafter referred to as a ‘neutral experience’), or a single bout of immobilization stress (hereafter referred to as a ‘negative experience’) all elicited comparable levels of ChR2-mCherry expression in the dentate gyrus (FIG. 6A-E).

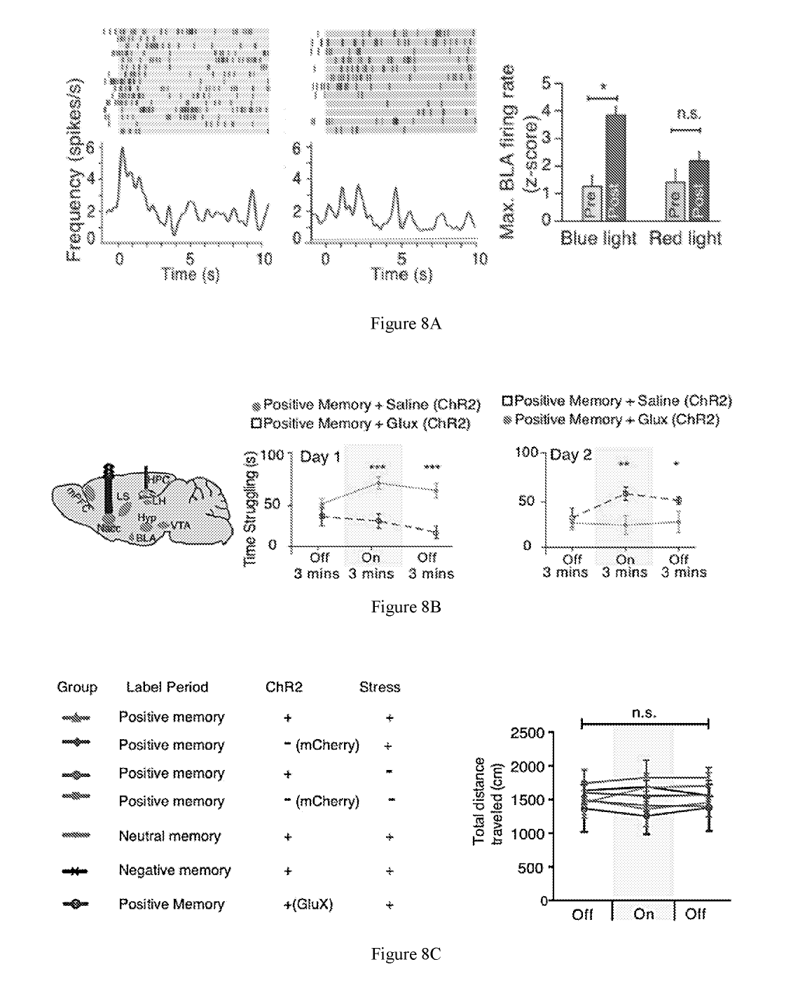

As shown in FIG. 1A, mice were split into six groups (see Methods). After 10 days of chronic immobilization stress (CIS) (FIG. 6F) or in a home cage, all groups were put through the open field test (OFT) and elevated plus maze test (EPMT) as measures of anxiety-like behaviors, as well as the tail suspension test (TST) as a measure of active/passive escape behavior in response to a challenging situation, and the sucrose preference test (SPT) for anhedonia (10-14). In unstressed animals, optogenetic reactivation of cells previously active during a positive experience did not significantly change anxiety-related measures, time spent struggling, or preference for sucrose compared to unstressed mCherry controls (FIG. 1B-E). In the stressed groups, the CIS paradigm elicited a robust decrease in time struggling and preference for sucrose, as well as increased anxiogenic responses, consistent with previous reports (13,14) (FIG. 1B-E). However, optically reactivating dentate gyrus cells that were previously active during a positive experience, but not a neutral or a negative experience, in stressed animals acutely increased time struggling and sucrose preference to levels that matched the unstressed group's behavior (FIGS. 1B, C). Additionally, optical reactivation of dentate gyrus cells associated with a positive experience decreased the latency to feed in a novelty-suppressed feeding test (NSFT) (14) (FIG. 7A) without affecting hunger or satiety (FIG. 7B). Once again, the CIS paradigm had an anxiogenic effect across all groups, and all groups failed to show light-induced behavioral changes in the OFT or EPMT (FIGS. 1D, E). Similarly, total distance travelled was consistent across groups (FIG. 8C). Taken together, these data support a conclusion that reactivating dentate gyrus cells labelled by a positive experience is sufficient to acutely reverse the behavioral effects of stress in the TST, SPT and NSFT.

To identify potential neural loci that mediate the light-induce reversal of the stress-induced behaviors observed in the experiments, all subjects first underwent the CIS protocol and then were exposed to the TST while dentate gyrus cells previously active during a positive experience were optically reactivated. A brain-wide mapping of c-Fos expression was then performed in areas activated by this treatment (FIG. 2A).

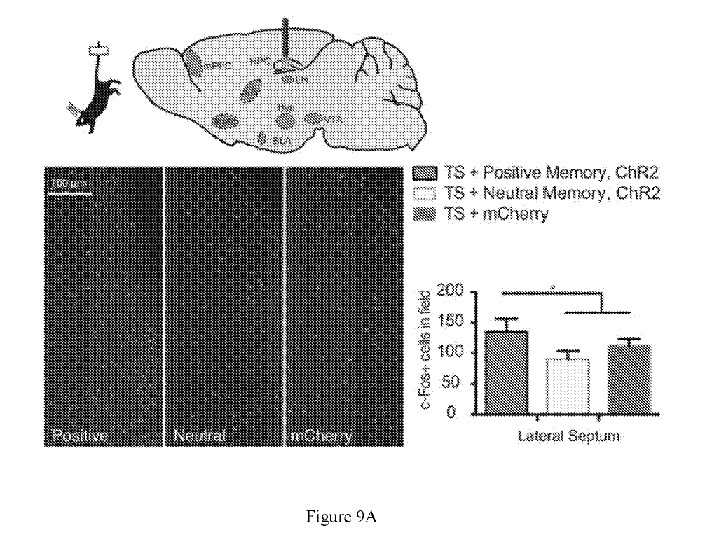

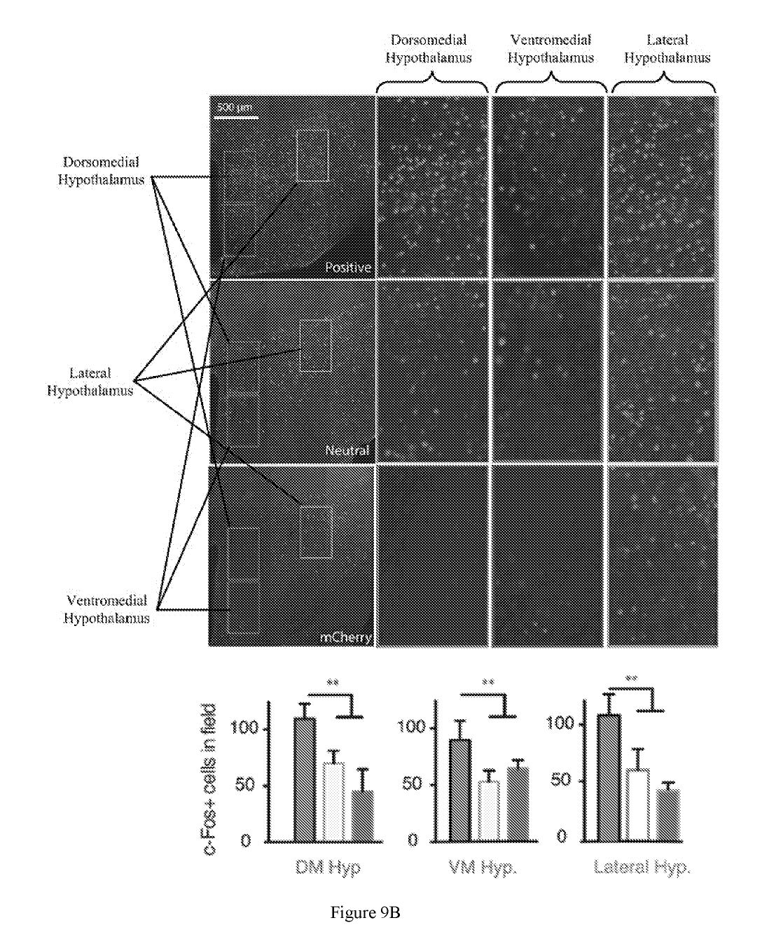

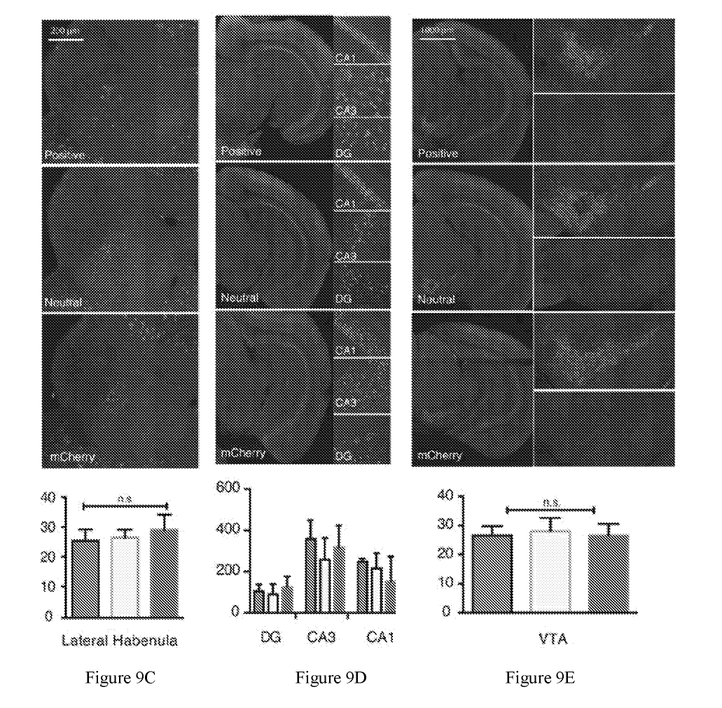

Optical reactivation of dentate gyrus cells labelled by a positive experience correlated with a robust increase of c-Fos expression in several brain areas, including the nucleus accumbens (NAcc) shell, lateral septum, basolateral amygdala (BLA), central amygdala, as well as the dorsomedial, ventromedial, and lateral hypothalamus (FIG. 2B-I and FIG. 9A,

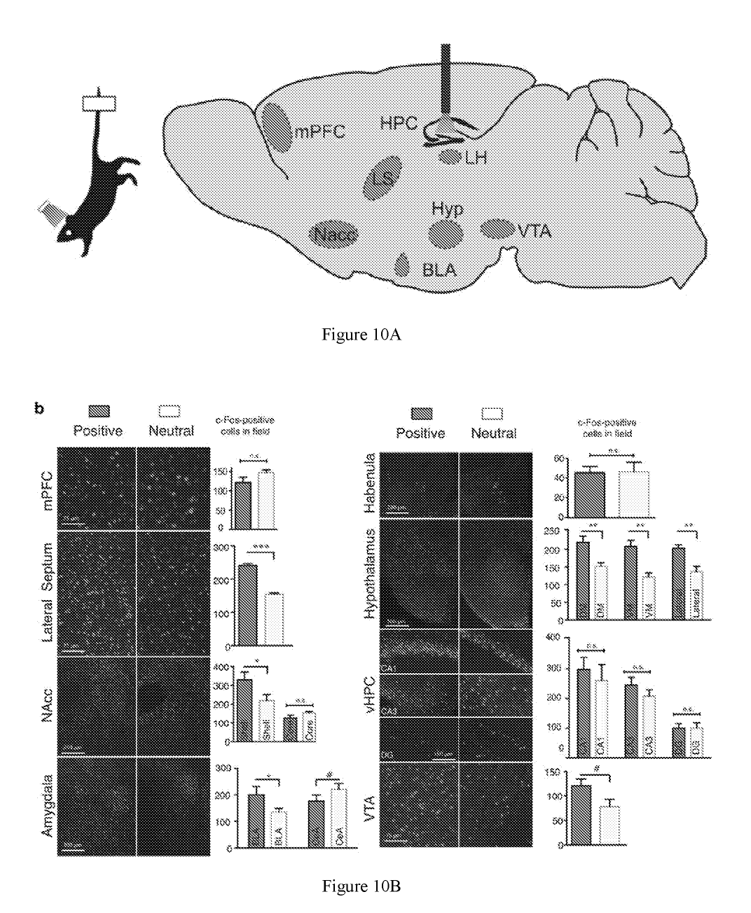

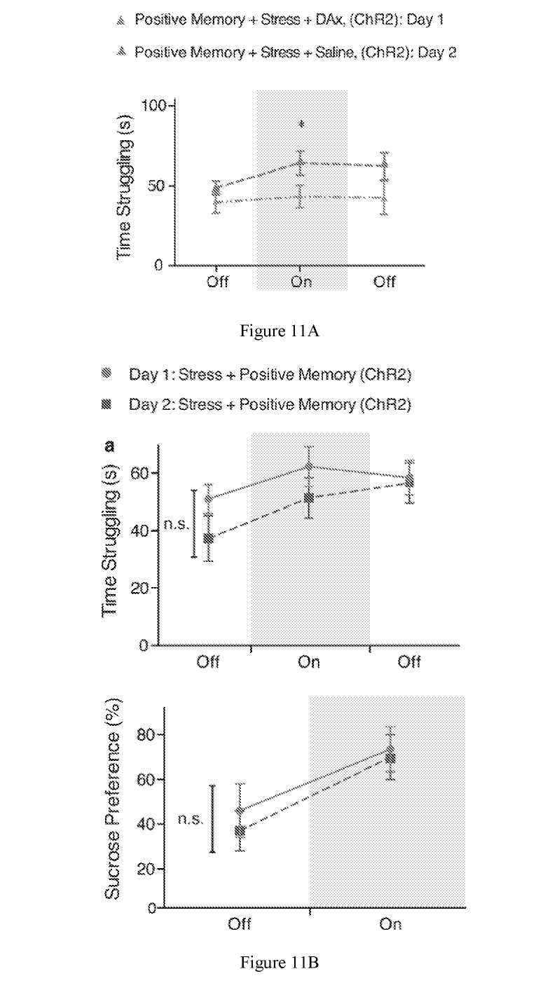

B), but not in the medial prefrontal cortex (mPFC) (FIG. 2J-M) or in several other loci (FIG. 9C-E). Furthermore, single-unit activity in the BLA of mice was monitored while simultaneously activating dentate gyrus positive memory engram cells with blue light and found that ˜8% of cells (9/106; n=3 mice) had excitatory (8/9 cells) or inhibitory (1/9 cells) responses (FIG. 8A). A parallel set of experiments in which unstressed animals received optical stimulation of dentate gyrus cells revealed mostly similar patterns of c-Fos expression (FIG. 10).

The NAcc has been heavily implicated in stress responses, mood disorders, and processing natural rewards (2,5,10-12,15-20). Moreover, pathophysiological dysfunction of the NAcc in response to various stressors has been implicated in anhedonia and reward conditioning (17-20). The within-subject experiments that were performed revealed that in the TST, the behavioral effects of optically reactivating dentate gyrus cells labelled by a positive experience were blocked in the group of mice that concurrently received the glutamate receptor antagonists NBQX and AP5 in the NAcc, but not in the group that received saline, without altering basal locomotion (FIG. 8B, C). Blocking dopaminergic activity yielded a similar blockade of the dentate gyrus light-induced effects (FIG. 11A).

The BLA is known to have robust glutamatergic inputs to the NAcc (19), and previous studies have implicated BLA projections to the NAcc in enabling reward-seeking behavior (19). Studies were performed to examine whether the hippocampus (dentate gyrus)-BLA-NAcc functional pathway is crucial for the real-time light-induced rescue of depression-related behavior. In these studies, the transgenic mice were bilaterally injected with TRE-ArchT-eGFP into the BLA to allow for activity dependent ArchT-eGFP labelling of axonal terminals from the BLA to the NAcc in response to a positive experience (21) (FIGS. 3A, B). Optic fibres were bilaterally placed over the NAcc and the dentate gyrus to allow for real-time inhibition of these terminals originating from ˜18% (FIG. 3C) of BLA neurons and simultaneous activation of ChR2-mCherry positive dentate gyms cells, respectively, in stressed mice. At the neuronal level, light-induced reactivation of dentate gyrus cells previously activated by a positive experience also reactivated BLA (8) and NAcc (18), but not mPFC, cells (that is, endogenous c-Fos1 cells, red) previously activated by the same positive experience (that is, ArchT-eGFP1 cells, green) (FIG. 3C). These results suggest that the dentate gyrus engram cells are functionally connected to BLA engram cells and NAcc engram cells. At the behavioral level, inhibition of BLA terminals onto the NAcc blocked the dentate gyrus light-induced rescue in both the TST and SPT (FIG. 3D-G). Within the same behavioral session for the TST, and across 2 days for the SPT, when ArchT-mediated inhibition was released (that is, the green light was turned off), the rescue effects of reactivating dentate gyrus cells previously active during a positive experience were rapidly observed in all groups (FIG. 3D-G). ArchT-mediated inhibition of BLA-NAcc terminals alone did not negatively affect behavior in the TST or SPT beyond the levels of the stressed animals (FIG. 3D-G insets). The specificity of the hippocampus (dentate gyrus)-BLA-NAcc pathway for the rescue was supported by an analogous experiment conducted with bilateral injections of TRE-ArchT-eGFP into the mPFC. Although the mPFC is also known to provide robust glutamatergic input to the NAcc (19), the induction of c-Fos expression in this area upon optogenetic activation of dentate gyrus cells associated with a positive experience was not significantly higher than that observed with a neutral experience (FIG. 2), and mPFC cells reactivated by dentate gyrus cell reactivation was at chance level (FIG. 3C). Correspondingly, inhibition of terminals originating from ˜12% of the mPFC (FIG. 3C) onto the NAcc did not block the dentate gyrus light-induced rescue in either the TST or SPT (FIG. 3D-G). Moreover, inhibition of BLA, but not mPFC, terminals onto the NAcc partially inhibited the dentate-gyms-mediated, light-induced increase of c-Fos⁺ cells observed in the NAcc shell (FIG. 3H), supporting the conclusion that the hippocampal (dentate gyrus)-BLA-NAcc pathway of positive engrams plays a crucial role in the rescue of depression-related behavioral phenotypes.

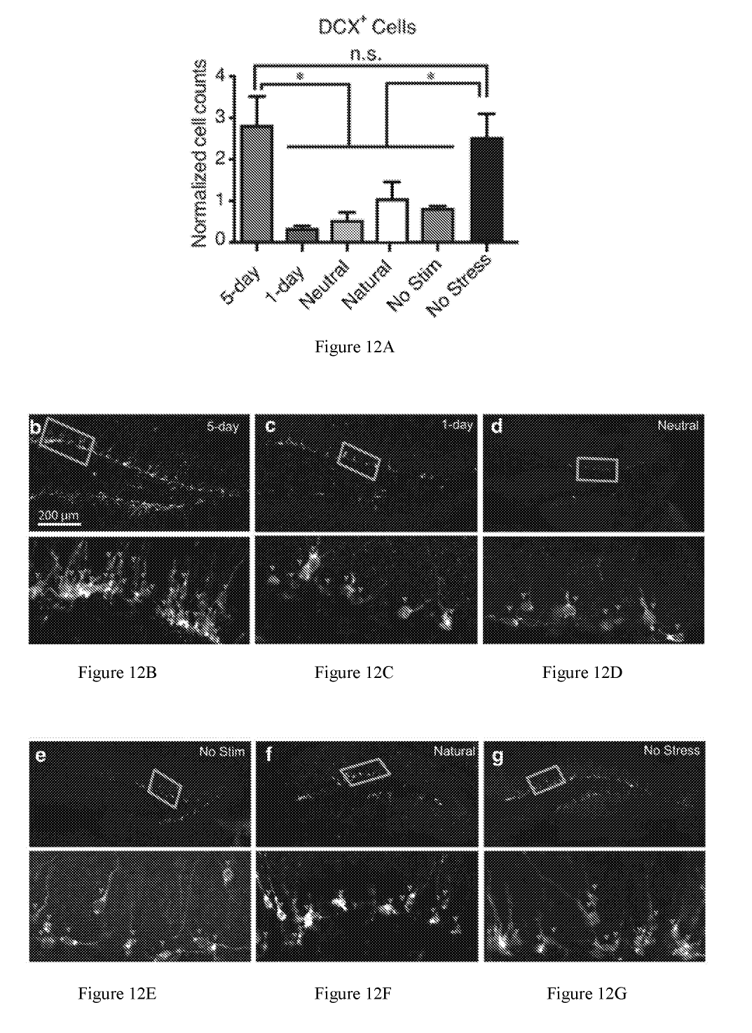

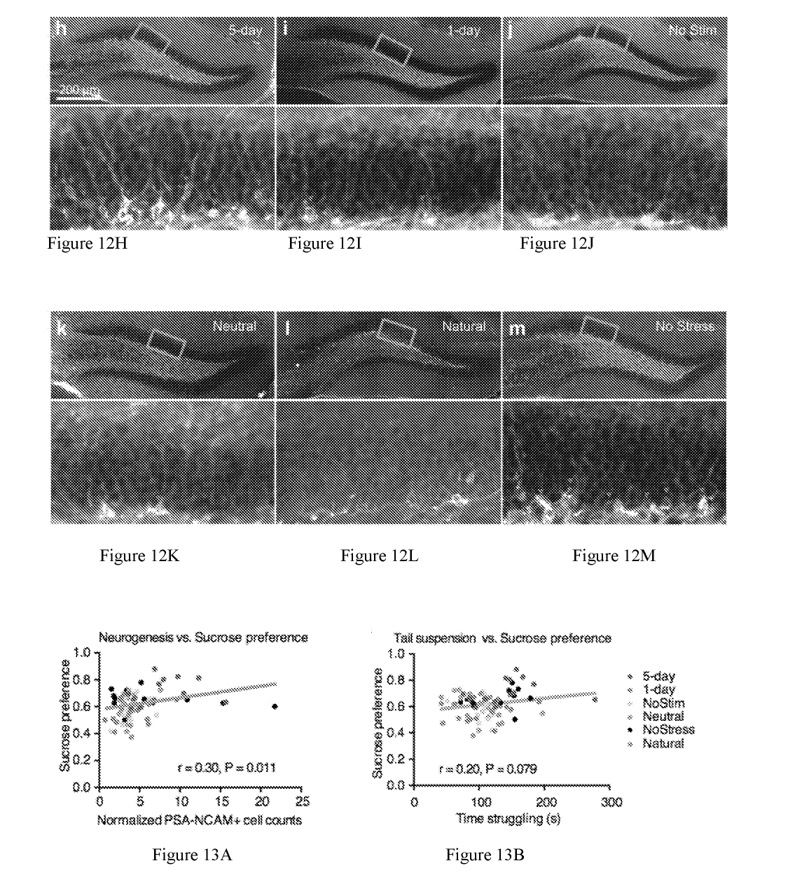

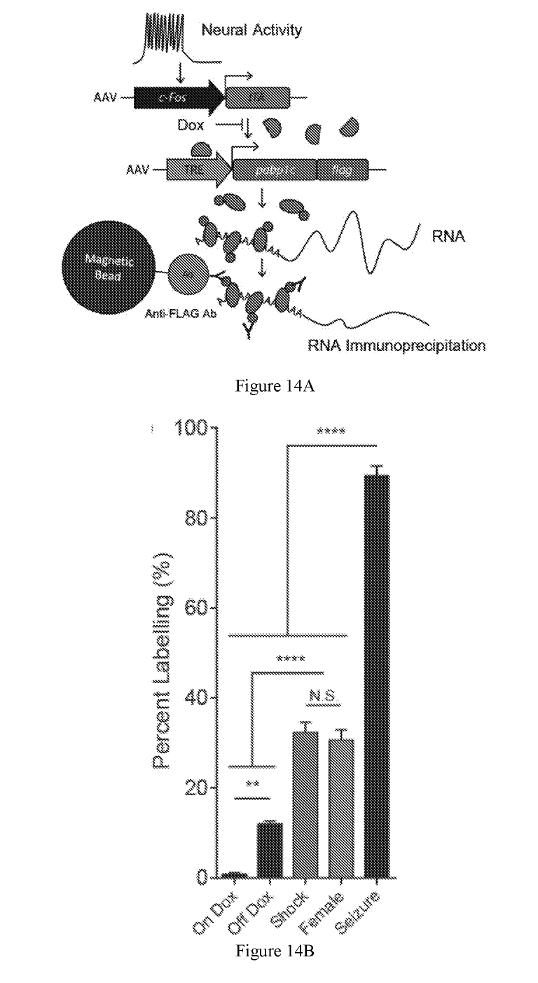

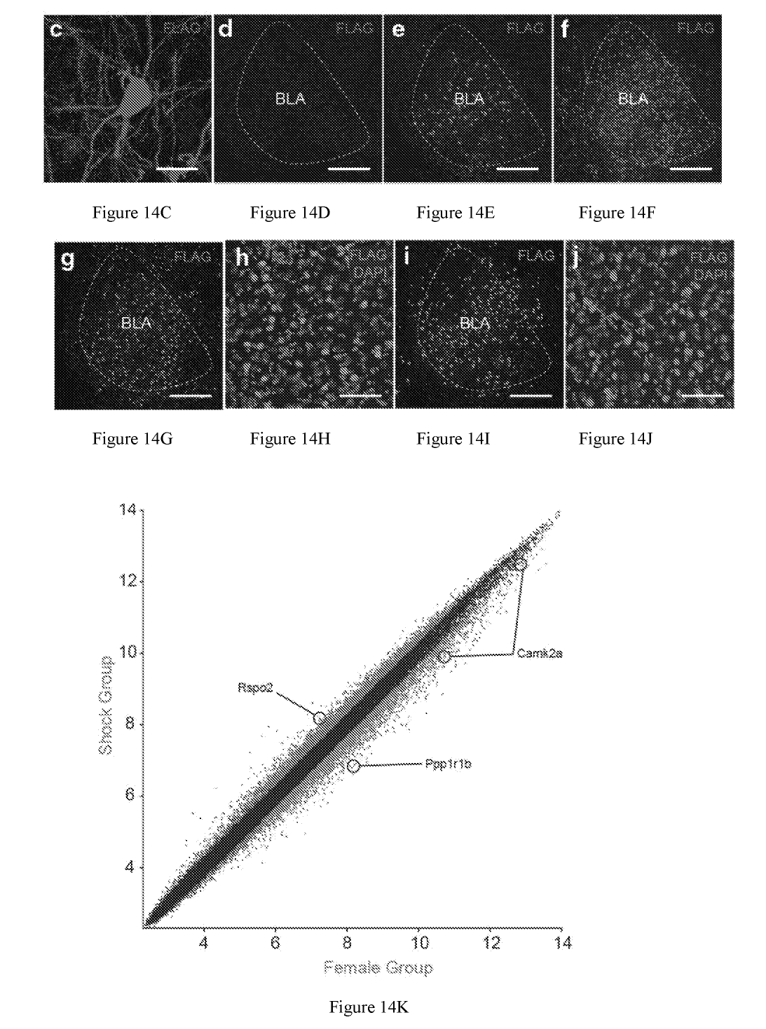

Recent meta-analyses have suggested that treating psychiatric disorders through prescribed medication or cognitive interventions are capable of producing symptom remission when administered chronically (20), though the neural underpinnings inducing and correlating with long-lasting rescues are poorly understood (20,22,23). The aforementioned acute intervention did not induce enduring behavioral changes (FIG. 11B). As series of experiments were conducted to investigate whether chronic reactivation of dentate gyrus engram cells could attenuate depression-related behaviors in a manner that outlasted acute optical stimulation following the protocol depicted in FIG. 4A (Methods). A group in which dentate gyrus cells associated with a positive experience were optically reactivated across 5 days, but not 1-day or no stimulation groups, showed a reversal of the stress-induced behavioral deficits measured in the TST and SPT that was not significantly different from an unstressed control group (FIGS. 4B, C). A group in which dentate gyrus cells associated with a neutral experience were optically reactivated across 5 days did not show such effects, nor did a group that was exposed to a natural social reward for 5 days (FIG. 4B-D). Histological analyses revealed decreased levels of neurogenesis as measured both by the polysialylated neuronal cell adhesion molecule (PSA-NCAM) and doublecortin (DCX)—often considered markers of developing and migrating neurons (24,25)—in all stressed groups except for the positive experience and 5-day stimulation group, and the unstressed control group (FIG. 4D and FIG. 12). This increase in adult-born neurons positively correlated with the degree to which each group preferred sucrose in the SPT (FIG. 13A); moreover, performance levels on the SPT and TST positively correlated with one another on an animal-by-animal basis (FIG. 13B).

The resulting data demonstrate that the depression-related readouts of active/passive coping-like behavior and anhedonia, as measured in the TST and SPT, respectively, can be ameliorated by activating cells in the hippocampus associated with a positive memory, while anxiety-related behaviors measured by the OFT and EPMT remained unchanged. Differential regulation of depression- and anxiety-related behavior could have been achieved by leveraging the functional segregation present along the hippocampus dorsal-ventral axis; for instance, activation of ventral hippocampal dentate gyrus engram cells could reveal heterogeneous, behaviorally relevant roles in the emotional regulation of anxiety and stress responses that our dorsal hippocampus manipulations presumably did not access (26,27). The results support a conclusion that, at the engram level, the circuitry sufficient to modulate anxiety-related behavior relies more heavily on a synaptic dialogue within the amygdala, its bidirectional connections with the ventral hippocampus, and its effects on downstream mesolimbic and cortical structures (10,11,26,27).

Depression is diagnosed as a constellation of heterogeneous symptoms; their complex etiology and pathophysiology underscore the varied responses to currently available treatments. While most psychopharmacological treatments take weeks to achieve effects, other alternative treatments such as deep-brain stimulation and the NMDA antagonist ketamine have been reported to have rapid effects in a subset of patients (28). In rodents, optogenetic stimulation of mPFC neurons, mPFC to raphe projections, and ventral tegmental dopaminergic neurons achieved a rapid reversal of stress-induced maladaptive behaviors (4,10,11). The results obtained support a conclusion that the acute behavioral changes observed reflected the degree to which directly stimulating positive-memory engram-bearing cells might bypass the plasticity that normally takes antidepressants weeks or months to achieve, thereby temporarily suppressing the depression-like state. In support, it was observed that the effects of optically stimulating a positive memory are contingent on active glutamatergic projections from the amygdala to the NAcc in real time, as well as intra-NAcc dopamine activity (18). The data obtained in the experiments described herein, dovetail with this circuit's proposed role of relaying BLA stimulus-reward associations to a ventral striatal motor-limbic interface. This interface is thought to be capable of coalescing such information with motivational states and finally translating such activity into behaviorally relevant outputs (5,17-19).

Moreover, it was determined that the chronic stimulation data supports a conclusion that repeatedly activating dentate gyrus engram cells associated with a positive experience elicits an enduring reversal of stress-induced behavioral abnormalities and an increase in neurogenesis.

The experimental results described herein support a causal link between chronically reactivated positive memory engrams and the corresponding rescue of behaviors, and suggests various mechanisms that may be involved such as: a normalization of VTA firing rates (29), epigenetic and differential modification of effector proteins (for example, CREB, BDNF) in areas upstream and downstream of the hippocampus (30), and a reversal of neural atrophy in areas such as CA3 and mPFC or hypertrophy in BLA (26). The aforementioned molecular and homeostatic mechanisms—in light of the observation in this study that there was an increase of adult-born neurons in the 5-day stimulation group-could be partly realized in a hormone- or neuromodulator-mediated manner (FIG. 9). Finally, the results of the studies described herein indicate that exposing stressed subjects to a natural positive experience repeatedly is not effective, while repeated direct reactivations of dentate gyrus engram cells associated with a previously acquired positive memory is effective (FIG. 4B-D). the results support the effectiveness of invasively stimulating these dentate gyrus cells as an effective means to activate both the internal contextual representation associated with a positive experience as well as associated downstream areas, while exposure to natural exogenous positive cues may not be able to access similar neural pathways in subjects displaying depression-like symptoms such as passive behavior in challenging situations and anhedonia (FIG. 4B-D).

Collectively, the data described here build a bridge between memory engrams in the brain and animal models of psychiatric disorders. The results support a conclusion that direct activation of dentate gyrus engram cells associated with a positive memory offers a potential therapeutic node for alleviating a subset of depression-related behaviors and, more generally, that directly activating endogenous neuronal processes may be an effective means to correct maladaptive behaviors.

### Example 2

### Methods

### Subjects.

Wild-type C57BL/6J (Stock #000664), mice were obtained from Jackson Laboratory.Cartpt-Cre (Stock #036659-UCD), produced through the GENSAT project, was obtain from Mutant Mouse Resource and Research Center (MMRRC). Cartpt-Cre mice were backcrossed to C57BL/6J for 2 generations. Rspo2-cre mice was generated using a bacterial artificial chromosome (BAC) clone (RP32-39M21) with a Cre construct driven by the regulatory elements of Rspo2. Experiments were performed in mice 8-16 weeks of age. All subjects were male mice. All subjects were cared and maintained in accordance with protocols approved by the Massachusetts Institute of Technology (MIT) Committee on Animal Care (CAC) and guidelines by the National Institutes of Health (NIH).

### Viruses.

The mouse minimal fos promoter (−623 to +1050 from the transcriptional start site) followed by advanced tTA was cloned into an adeno-associated virus (AAV) backbone to generate the pAAV-cfos-tTA vector. cDNA clone of mouse Pabpc1 with a C-terminal Myc-DDK tag (Origene, Cat. #MR209653) was subcloned into the pAAV-TRE-ChR2-EYFP plasmid to generate the pAAV-TRE-PABP-FLAG plasmid. AAV plasmids were packaged into AAV9 vectors by the Gene Therapy Center and Vector Core at the University of Massachusetts Medical School. AAV5-Ef1a-DIO-eArch3.0-eYFP (AV5257), AAV5-Ef1a-DIO-ChR2-eYFP (AV5226B), and AAV5-Ef1a-DIO-eYFP (AV4310D) was obtained from University of North Carolina at Chapel Hill Vector Core. AAV9-Ef1a-ChR2-eYFP (CS00633-3CS) was obtained from University of Pennsylvania School of Medicine Vector Core.

### Stereotactic Injections.

Subjects undergoing stereotactic injections were anaesthetized under isoflurane. Standard stereotactic procedures were used. Viruses were injected using a mineral oil filled glass micropipette attached to a 1 μL microsyringe. For activity-dependent transcriptional profiling, 200 nL of ˜2.0×109 GC of AAV9-cfos-tTA and AAV9-TRE-PABP-FLAG (1:1 mixture) were bilaterally injected into the BLA (distance from bregma, AP−1.4 mm, ML±3.3 mm, DV−4.85 mm) of doxycycline (Dox) fed mice and incubated for 7 days prior downstream experiments. For behavioral experiments, 200 μL of viral stocks of AAV5 cre-dependent viruses was injected into the BLA of Rspo2-cre (AP−1.6 mm, ML±3.3 mm, DV−4.85 mm) and Cartpt-cre mice (AP−2.0.mm, ML±3.4 mm, DV−4.9 mm) and incubated for 3-4 weeks prior to behavioral experiments. For retrograde tracing, Alexa Fluor 555-conjugated cholera toxin subunit B (CTB) (1 μg/μL) was unilaterally injected into the CeC (50 nL, AP−1.0, ML+2.9, DV−4.5), CeL/M (100 nL, AP−1.34 mm, ML+2.9 mm, DV−4.6), NAc (300 nL, AP+1.0, ML+0.75, DV−4.8) and incubated for 7 days prior to sacrifice. Alexa Fluor 555 and 647-conjugated CTB was injected into the PL (200 nL, AP+1.75, ML+0.3, DV−2.3) and IL (200 nL, AP+1.75, ML+0.3, DV−3.0) and incubated for 10 days prior to sacrifice. For brain slice electrophysiological experiments, 200 nL of AAV9-Ef1a-DIO-ChR2-eYFP was injected into the BLA of 4-5 week old Rspo2-cre and Cartpt-cre mice and incubated 4 days prior to electrophysiological experiments.

### Fiber Implantation.

5.0 mm Mono fiberoptic cannulas (Doric Lens) was implanted (unilaterally or bilaterally, depending on the experiment) above the BLA of Rpso2-cre and Cartpt-cre (AP−2.0 mm, ML+3.3 mm, DV−4.3), and above the NAc of Rspo2-cre (AP+1.3 mm, ML+0.75 mm, DV−4.0). Once positioned above the BLA, the mono fiberoptic cannula was cemented using dental cement (Teets cold cure; A-M Systems) to the skull, which contained 2 screws that lied medially to the implant site. Once the dental cement cured, a protective cap surrounding the implant, made using a 1.5 mL black Eppendorf tube, was fixed onto the implant using dental cement. Mice spent 3-4 week post-operation for recovery. Mice were handled by investigator 2-3 days prior to behavioral experiments.

### RNA Immunoprecipitation.

12 wild-type male mice kept on Dox diets were bilaterally injected with AAV9-c-fos-tTA and AAV9-TRE-PABP-FLAG virus. One week post-operation, mice were taken off a Dox diet for 2 days and underwent a fear conditioning protocol (3 shocks, 0.75 mA, 2 s duration) or exposed to a female mouse in the home cage for 2 hrs. Immediately after, mice were returned to a Dox diet. 2 days later, mice were anaesthetized with isoflurane and were sacrificed by decapitation. 2 control mice were kept on a Dox diet. Brains were dissected, flash frozen on dried ice, and stored in −80° C. until RNA immunoprecipitation. RNA immunoprecipitation was performed in a similar fashion as described by the McKnight Lab (University of Washington). Brains were thawed for 30 min in a −16° C. cryostat; 300 μm sections across the BLA were collected. Using a razor blade, the BLA's was crudely dissected 2 mice brains and were collected into a single 1.5 mL microcentrifuge tubes. This yielded ˜30 μg of brain tissue. 1 mL of homogenization buffer (HB, 1% NP-40, 100 mM KCl, 50 mM Tris pH 7.4, 12 mM MgCl2, 200 U/mL Promega RNasin, 1 mM DTT, 100 μg/mL cyclohexamide, 1 mg/mL heparin, 1% protease inhibitors (P8340, Sigma)). Samples were transferred to a 2 mL dounce homogenizer and homogenized using pestle A and, subsequently, pestle B. Homogenized samples were transferred to 1.5 mL microcentrifuge tube and were centrifuged at 10,000 rcf. Supernatant was separated into a new microcentrifuge tube, 5 μL of anti-FLAG (F7425, Sigma) was added and incubated for 6 hrs at 4° C. 200 μL Pierce A/G Magnetic Bead were washed in HB, added to the homogenates, and incubated overnight at 4° C. Magnetic beads were separated using a magnetic tube rack and washed 3 times in a salt buffer (0.3M KCl, 1% NP-40, 50 mM Tris pH 7.4, 12 mM MgCl₂, 100 μg/mL cyclohexamide, 0.5 mM DTT). Protein-RNA complexes were dissociated from magnetic bead by vortexing samples in lysis buffer (RLT lysis buffer from Qiagen RNease Kits with 10 μL/mL β-Mercaptoethanol). Magnetic beads were drawn off and RNA was isolated using the Qiagen RNAeasy Micro Kit. RNA samples were stored in −80° C. until further downstream experiments.

### RNA Analysis.

RNA samples were analyzed using the Affymetrix Mouse 430 2.0 chip by MIT BioMicroCenter. CEL files from the Mouse 430 2.0 chip were normalized by RMA or MAS5 through the Affymetrix Expression Console Software. Subsequently, CHP files were analyzed through Affymetrix Transcriptome Analysis Console 2.0-3 samples from the shocked mice and 3 samples from the female exposed mice were grouped. The data from this analysis has been deposited to the NCBI Gene Expression Omnibus (GEO), accession number GSE78137.

### Screening and Selecting BLA Gene Marker Candidates.

Based on the data obtained from the array, the top gene candidates, independent of statistical significance, enriched in either the RMA or MAS5 normalized data set were screen on Allen Mouse Brain Expression Atlas (http://mouse.brain-map.org/). Based on expression patterns in the BLA, 16 gene candidates that were enriched in the shock group were selected—Acvr1c, Cdh9, Crhbp, Gabra1, Gabra2, Gria4, Htr2c, Htr3a, Nptx2, Nrxn3, Pth1h, Pcdh18, Rspo2, Sema5a, Slc30a1, Zfpm2. Based on expression patterns in the BLA, 21 gene candidates that were enriched in the female group were selected-Adrbk1, Aig1, Esrra, Gipc1, Gpr39, Gpr137, Gpr165, Gria1, Grin1, Oprl1, Neurl1a, Nos1, Nos1ap, Ntrk3, Ntng2, Penk, Ppplrlb, Slc24a4, Slc30a3, Stx1a, Synpo. Interneuronal markers—Calb1, Npy, Sst, Vip, Pvalb—and pyramidal cell markers—Camk2a, Thy1—were selected as positive controls.

### Tissue.

For the screening of candidate gene markers, wild-type mice 12-16 week old were anaesthetized with isoflurane and were sacrificed by decapitation. Brains were quickly dissected and immediately flash frozen on aluminum foil on dried iced and stored in −80° C. A single session of sectioning consisted of 12 wild-type brains and 60 Superfrost Plus slides (25×75 mm, Fisherbrand). 30 min prior to sectioning, brains were equilibrated to −16° C. in a cryostat. Brains were serially sectioned coronally at 20 μm and thaw-mounted onto slides. Each mouse brain produced one section on each of 60 slides—sections from AP−0.8 mm to AP−2.0 mm were taken from each brain. Sections from each subsequent brain started in a staggered fashion (begun on the 6th, 11th, 16th, etc. slide). Therefore, each slide resulted with 12 coronal brain sections representing 0.1 mm intervals between AP−0.8 mm to AP−2.0 mm. Brains were dried at room temperature for 30 min prior to storage at −80° C. In order to obtain a homogenous representation of the BLA, no more than 2 sections were lost during sectioning of a single brain. For single molecule fluorescent in situ hybridization, mouse brains were collected through the flash frozen method (as described above). Using a cryostat, an individual brain was serially sectioned and thaw-mounted onto Superfrost Plus slides. Coronally cut brain slides were serially sectioned at 20 μm onto 10 slides, each slide contained 11 to 12 brain sections, spaced 0.2 mm apart spanning AP−0.8 mm to AP−2.8 mm. Sagittally cut brain slides was serially sectioned at 20 μm onto 8 slides, each slide contained at least 12 brain sections, spaced 0.16 mm apart spanning ML3.8 mm to ML2.8 mm. Slides were dried at room temperature for 60 min prior to storage at −80° C. For immunohistochemistry, mice were euthanized by avertin overdose, perfused with 1× phosphate-buffered saline (PBS) and 4% paraformaldehyde. Brains were dissected and stored in 4% paraformaldehyde at room temperature for 8 to 12 hrs, prior to storage in 1×PBS at 4° C. Coronally cut brains were sectioned at 50 μm on a vibratome and serially collected into 4 wells. Each well contained coronal sections spaced 0.2 mm apart.

### Fluorescent In Situ Hybridization.

Single-label fluorescent in situ hybridization (FISH) was performed using RNA probes generated from the pCRII-TOPO Vector (ThermoFisher). pCRII-TOPO vector, cut with EcoRI, was used as the backbone for cloning cDNA of candidate gene markers. Mouse brain cDNA was obtained via reverse transcription using the Omniscript RT kit (Qiagen) of RNA extracted from mouse brains (Qiagen RNeasy Lipid Tissue Mini Kit). PCR primers were the same as the forward and reverse primers reported on Allen Mouse Brain Expression Atlas with the addition of 5′-cagtgtgctggaatt-3′ (SEQ ID NO: 1) and 5′-gatatctgcagaatt-3′ (SEQ ID NO: 2) to the 5′ end of the forward primer and reverse primer, respectively. PCR products for each candidate gene was isolated and cloned into the pCRII-TOPO backbone using TOPO cloning (Clontech In-Fusion HD) and maintained in Stellar Competent Cells (Clontech). RNA probes were generated by cutting pCRII-TOPO plasmids with HindIII and transcribing the anti-sense strand using a T7 RNA polymerase (NEB, HiScribe T7 High Yield RNA Synthesis Kit) with digoxygenin-labelled UTP (Roche). Digoxygenin-labelled anti-sense RNA probes were isolated (Qiagen, RNeasy Mini Kit) and stored in −80° C.

Tissue preparation of single label FISH was performed similar to standard mouse brain FISH protocols. On day 1, slides were fixed in 4% paraformaldehyde at 4° C., washed twice in phosphate buffer pH 7.4, rinsed in diethylpyrocarbonate (DEPC) water and tetraethanolamine (TEA) buffer, pretreated with acetic anhydride, washed in 2× saline-sodium citrate solution (SSC), washed in increasing concentrations of ethanol (70%, 95%, 100%), delipidated with chloroform, washed with decreasing concentrations of ethanol (100%, 95%). The probes were dried for 2 hrs at room temperature. RNA probes were denatured at 100° C. then cooled on ice, then were applied onto slides in a solution of hybridization buffer and tRNA and coverslipped for overnight incubation at 60° C. Day 2, slides underwent post-hybridization stringency washes—2× Saline-Sodium Phosphate-EDTA buffer (SSPE), 50% formamide in 2×SPPE at 60° C., and twice in 0.1×SSPE at 60° C. Endogenous peroxidase activity was removed with 0.3% hydrogen peroxide in Tris-NaCl-Tween (TNT) buffer, followed by 3 washes in TNT buffer. Slides were blocked in TNT buffer with blocking reagent (PerkinElmer) (TNB) prior to being incubated with 1:100 peroxidase-conjugated anti-digoxygenin FAB fragment (anti-dig-POD, Roche) for 2 hrs at room temperature. Anti-dig-POD was removed by a series of 3 TNT washes. Alexa 594-conjugated tyramide signal amplification (TSA) solution was applied over the slides for 30 min, and then washed away with a series of 3 TNT washes. Slides were coverslipped and mounted using VectaShield mounting solution containing DAPI (Vector Laboratories).

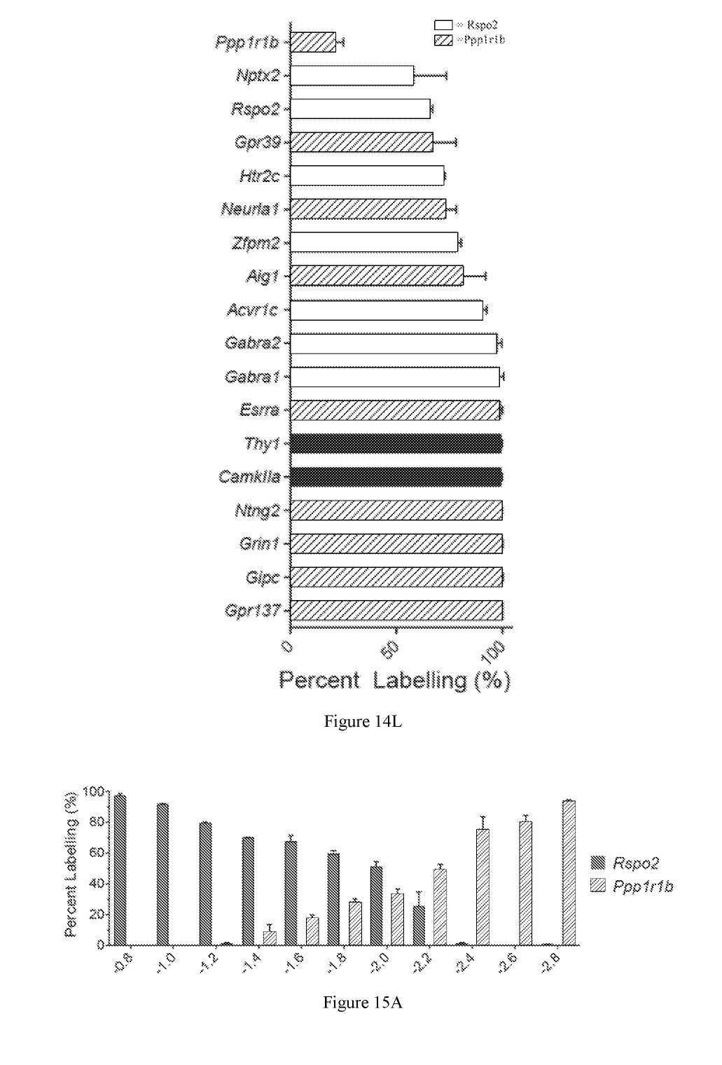

Percent labelling was calculated as the number of Gene+ cells as a percentage of the total large (<10 μm) DAPI+ BLA cells, which is an indirect indicator of BLA principle cells as shown from quantification of Camk2+(FIG. 14L).

### Single Molecule Fluorescent In Situ Hybridization.

Single molecule fluorescent in situ hybridization (smFISH) was performed using RNAscope Fluorescent Multipex Kit (Advanced Cell Diagnostics). Custom C1 and C2 DNA oligo probes were designed for Rspo2, and Ppplrlb. Camk2a., and Gad1 probes were available on the Advanced Cell Diagonstics Catalog. Brain sections were fixed in 4% paraformaldehyde for 15 min, and then washed in 50%, 70%, 100%, 100% ethanol for 5 min each. Slides were dried for 5 min. Proteins were digested using protease solution (pretreatment solution 3) for 60-90 s on wild-type tissues, 30 s on CTB expressing tissues, and 5-10 sec on EYFP expressing tissues. Immediately following, slides were washed twice in PBS. In parallel, C1 and or C2 probes were heated in a 40° C. water bath for 10 min. Probes were applied to the slides, coverslipped, and placed in a 40° C. humidified incubator for 3 hrs. Slides were rinsed twice in Rnascope wash buffer, and then underwent the colorimetric reaction steps according to standard kit protocol using AMP4A (C1-green, C2-red) or AMPB (C1-red, C2-green) depending on the color combination of choice. After the final wash buffer, slides were immediately coverslipped using ProLong Diamond Antifade mounting medium with DAPI (ThermosFisher).

### Immunohistochemistry.

Free floating brain sections were washed in PBST (1×PBS, 3% TritonX) 3 times for 10 min, blocked for 1 hour in blocking buffer (PBST, 5% normal goat serum), incubated in primary antibody in blocking buffer overnight at 4° C. Next day, brains were washed in 3, 10 min washes of PBST and incubated in secondary antibody in blocking buffer at room temperature for 2 hrs. Primary antibodies used were rabbit anti-FLAG (F7425, Sigma, 1:1000), chicken anti-GFP (Invitrogen, A10262, 1:1000), rabbit anti-fos (Santa Cruz, sc-52, 1:2000). Secondary antibodies used were goat anti-chicken Alexa Fluor 488 (Invitrogen, A11039, 1:1000), goat anti-rabbit Alexa Fluor 555 (Invitrogen, A21428, 1:1000). After 3 additional 10 min PBST washes, slides were coverslipped and mounted using VectaShield mounting solution containing DAPI (Vector Laboratories). For immunohistochemistry of electrophysiological experiments, brain slices were washed in PBST 3 times for 10 min, blocked for 1 hour, and then incubated with chicken anti-GFP (Invitrogen, A10262, 1:1000), to visualize ChR2-eYFP fibers, and CF555 Streptavidin (Biotium, 29038, 1:100), to visualize recorded neurons, overnight at 4° C. Next day, slices were washed in PBST 3 times for 10 min, and then incubated in chicken anti-GFP (Invitrogen, A10262, 1:1000) for 2 hrs at room temperature. After 3 additional 10 min PBST washes, slides were dried at room temperature for at least 6 hrs prior to coversliping and mounting using VectaShield mounting solution containing DAPI (Vector Laboratories).

### Microscopy and Histological Representation.

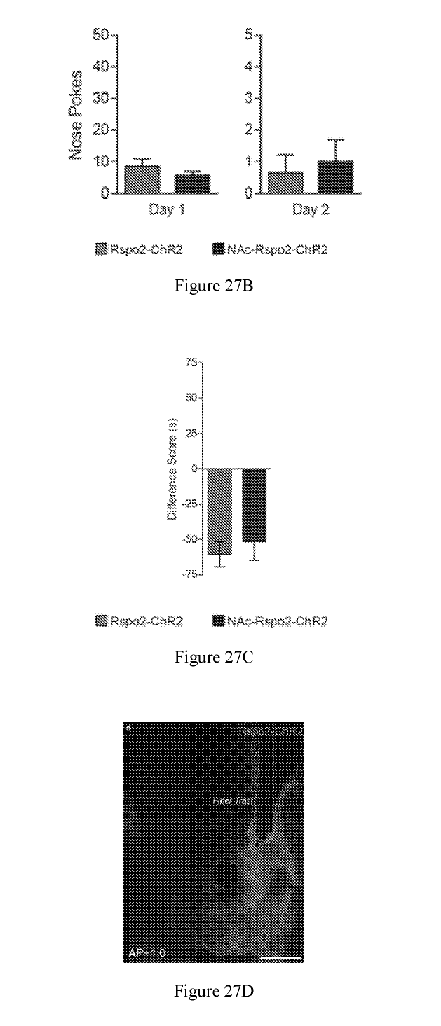

Micrographs were obtained using a Zeiss fluorescent microscope or Zeiss AxioImager M2 confocal microscope using Zeiss ZEN (black edition) software. Main figure representations were colored green for Rspo2⁺ neurons and red for Ppplrlb⁺ irrespective of native fluorescent labelling. CTB labeling were distinctly colored in order for visually distinguish different CTB experiments (FIG. 27).

c-Fos Experiment.

Wild-type mice were handled by investigator once each day for 3 days prior to stimulus exposure. Stimulus exposure experiments occurred within 1 hour of the dark cycle. Shocked mice, were exposed to a fear conditioning chamber (Med Associates) for 500 s and received 3 footshocks (0.75 mA, 2 s duration), then returned to home cage where water and food were removed. Female exposed mice were transported in home cages to an experimental room and were exposed to a wild-type female mouse. Context-exposed mice were exposed to the fear conditioning chamber for 500 s then returned to the home cage, and water and food were removed. Odor exposed mice were transported in home cages to an experimental room; water and food were removed; and habituated for 4 hrs prior to odor exposure. 1 mL of TMT (10% TMT in dH₂O), 1 mL of Peanut Oil, or 1 mL of BA (0.25% benzaldehyde in 70% ethanol) were pipetted into the center of the home cage. Water deprived (overnight) wild-type mice were transported in home cages to an experimental room, food removed, and habituated for 4 hrs prior to hydration. A bottle of water, quinine water (0.05% quinine hydrochloride dihydrate), sucrose water (5% sucrose), or an empty water bottle without a spout, was carefully placed into the home cage. 90 min after initial exposure, mice were sacrificed using avertin overdose and perfused for immunohistochemical analysis of c-FOS. For smFISH of c-fos, mice underwent the same stimulus exposure protocol, but were sacrificed using the flash freezing method (described above) 15 min after end of the stimulus exposure or in the case of water exposure, 15 min after satiety, which took <5 min after water exposure. Background signals levels of c-FOS and c865fos/Rspo2 or Ppplrlb were adjusted on ZEN, and were exported into image files for quantification in a blind fashion.

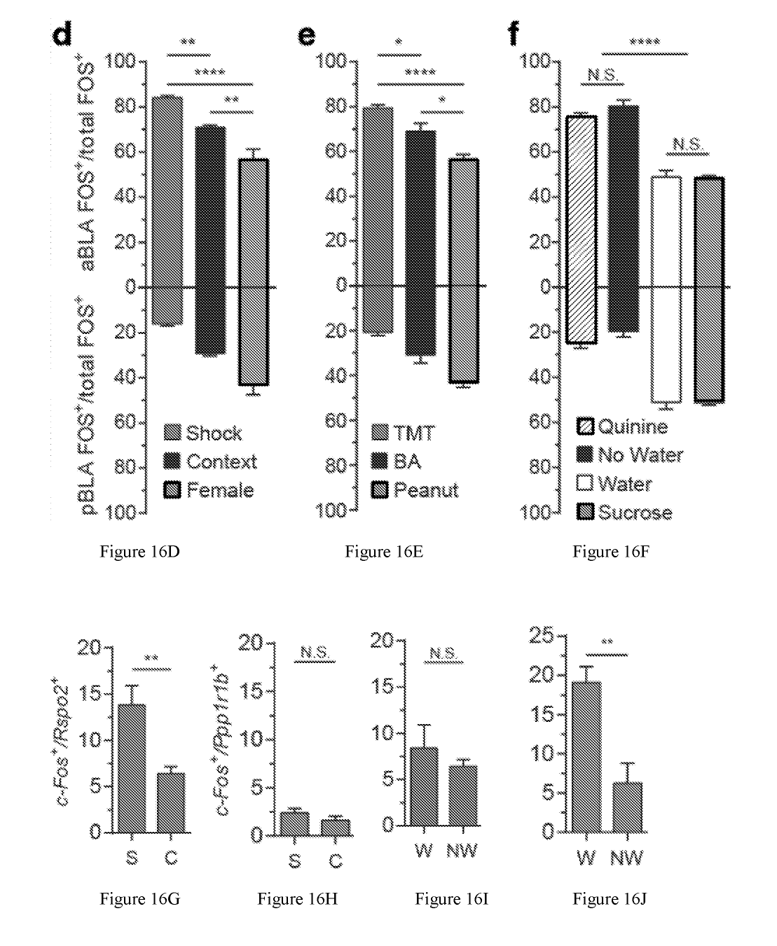

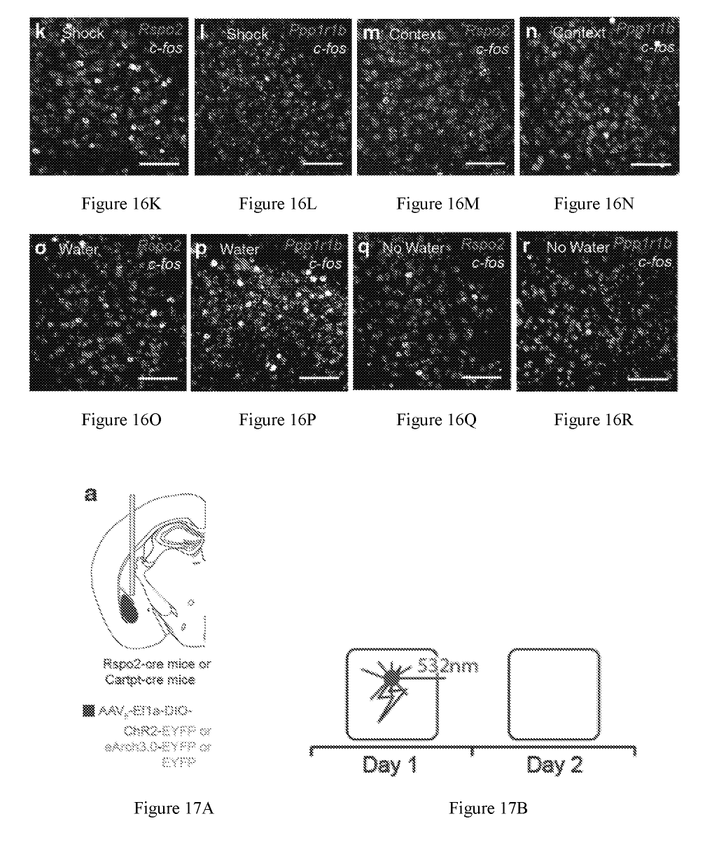

The relative c-FOS expression for the aBLA was calculated by (number of c-FOS⁺ cells in the aBLA)/(total number of c-FOS⁺ cells in the BLA), likewise the pBLA was calculated by (number of c-FOS⁺ cells in the pBLA)/(total number of c-FOS+ cells in the BLA). The aBLA and pBLA was determined using mouse brain altas boundaries of the aBLA and pBLA. The relative c-FOS expression of the aBLA and pBLA are mutually exclusive. Thus, when statistically comparing values between different conditions, significance values for comparison of the aBLA and comparison of the pBLA were redundant. Because of this, only the statistics for the aBLA was graphically represented (FIG. 16D-F). IHC C-FOS counting was performed for unilateral BLAs of 50 m sections. smFISH c-Fos counting was performed for unilateral BLAs of 20 m sections. The AP position was determined by a mouse brain atlas and was accurate within 1.2 mm.

### Fear Conditioning.

On day 1, mice were placed in to a fear conditioning chamber (Med Associates) while being bilaterally hooked up to optic fiber patchcords (Doric Lens) for 500 s and received shocks during the 198 s, 278 s, 358 s time point. For optical inactivation experiments, simultaneously with the shocks at the 198 s, 278 s, 358 s time points, a constant pulse of 532 nm light (10-15 mW) was delivered through the optical cannulas for duration of 20 s; for optical activation experiments, 20 Hz 473 nm (10-15 mW) light was used. On day 2, mice were hooked up to optic fiber patchcords and placed to the fear conditioning chamber for 180 s, where no shock or laser was delivered. Freezing behavior was scored manually using JWatchers1.0 in a blind fashion.

### Reward Conditioning.

Water-restricted mice were placed in an operant conditioning chamber (Island Motion) with one reward port equipped with a cue light. At the start of each trial, the onset of the cue light signals the availability of water reward contingent on a nose poke, lasting 5 s. Upon a successful nose-poke any time during the 5 s, a reward was immediately delivered through a water spout in the port and the cue light is turned off. A TTL triggering a laser pulse, bilaterally delivered to the implanted optic cannula, was issued at the same time as the reward delivery. For optical inactivation experiments, a constant 10 second pulse of 532 nm light (10-15 mW) was used; for optical activation experiments, a 10 s 20 Hz pulse train of 15 ms pulses of 473 nm light (10-15 mW) was used. During the following inter-trial interval of randomly distributed between 10 and 15 s, nose pokes did not elicit water rewards. Timestamps for cue light, nose pokes and reward deliveries were logged and analyzed with Matlab. Data arrays were constructed from the first rewarded trial to 150 trials thereafter. Total number of pokes was counted for the period. Percent in-port time during the cue presentation were calculated in 100 ms time bins and quantified with a z-score procedure (z=(x−μ)/σ) where x is the average percent time spent in the reward port, t and a are the mean and standard deviation of percent time spent in the reward port during the 5 s baseline period before cue onset.

### Optogenetic Freeze Test.

On day 1, mice were placed in to a fear conditioning chamber for 360 s. 20 Hz 473 nm light (10-15 mW) was unilaterally delivered through the optic cannula at the 180 s time point for 180 s. On day 2, mice were hooked up to fiber optic patchcords and returned to the fear conditioning chamber for 180 s without light stimulation. Freezing behavior was scored manually using JWatchers1.0 in a blind fashion.

### Optogenetic Self-Stimulation Test.

On day 1, food-deprived mice were placed into an operant conditioning chamber (Med Associates) equipped with a single nose port. The nose port contained a single food pellet in order to initiate the mouse into the port. 20 Hz, 473 nm light (10-15 mW, 5 s duration) was unilaterally delivered through the optic cannula contingent on a beam break in the nose port. Mice spend a total of 1 hour in the operant chamber. On day 2, mice were hooked up to fiber optic patchcords and returned to the reward conditioning chamber (with no food pellet) for 15 min without light stimulation. Total number of pokes was quantified by MEDPC (Med Associates) software on day 1 and day 2.

### Optogenetic Place Preference Test.

Mice were placed into the center of a rectangular box (70×25×31 cm) where each end of the box contained distinct wall cues. Immediately upon entry into the box, mice received continuous 20 Hz 473 nm light (10-15 mW) stimulation contingent on entry into a randomly pre-selected half of the box for 5 min. The position of the mice was tracked using EthoVision XT video tracking software (Noldus). The difference score (s) was calculated by (duration in the stimulated side)−(duration in the non-stimulated side). All behavioral experiments were performed by a set of mice in cohorts of 4-16 mice. Animals were selected for surgery and behavior in a pseudorandom fashion in that mice were, as much as possible, divided equally based on age and parents into experimental and control groups. For unilateral implants, mice received implants randomly and counterbalanced in the left or right hemisphere. For all behavioral experiments, two-tailed unpaired Student's t-test was performed between experimental groups and control groups. Mice lacking expression or misplaced fibers were excluded from analysis. Experimenters were blind during data analysis and whenever possible during the experimentation.

### Anatomical Experiments.

CTB experiments were performed as described above (Stereotactic injections). Percent labelling of CTB in the BLA was quantified by the number of CTB⁺ cells as a percentage of the total large (<10 μm) DAPI⁺ cells. For anatomical projection of BLA neurons, Rspo2-ChR2 and Ppplrlb-ChR2 mice tissue underwent immunohistochemistry for eYFP using an anti-GFP antibody to amplify the eYFP signal.

### Optogenetic Slice Electrophysiology.

Male mice (mean-PND 45 days) were anesthetized by isoflurane and their brains were dissected. By using a vibratome (VT1000S, Leica) 300 μm-thick parasagittal slices containing the basolateral amygdala were prepared in oxygenated cutting solution at ˜4° C. Slices were then incubated at −23° C. in oxygenated artificial cerebrospinal fluid (ACSF). The cutting solution contained 3 mM KCl, 0.5 mM CaCl₂, 10 mM MgCl₂, 25 mMNaHCO₃, 1.2 mM NaHPO₄, 10 mM d-glucose, 230 mM sucrose, saturated with 95% O/5% CO (pH 7.3, osmolarity 340 mOsm). The ACSF contained 124 mM NaCl, 3 mM KCl, 2 mM CaCl₂, 1.3 mM MgSO₄, 25 mM NaHCO₃, 1.2 mM NaHPO₄, 10 mM d-glucose, saturated with 95% O/5% CO (pH 7.3, osmolarity 300 mOsm). Slices were transferred into a submerged experimental chamber and perfused with oxygenated 36° C. ACSF at a rate of 3 ml min⁻¹.

Whole-cell recordings in current-clamp or voltage-clamp mode were performed by using an infrared differential interference contrast microscope (BX51, Olympus) with a water immersion 40× objective (N.A. 0.8), and equipped with four automatic manipulators (Luigs & Neumann) and a CCD camera (Orca R2, Hamamatsu). Borosilicate glass pipettes were fabricated (P97, Sutter Instrument) with a resistances of 3-5 MΩ, and filled with the following intracellular solution 110 mM potassium gluconate, 10 mM KCl, 10 mM HEPES, 4 mM ATP, 0.3 mM GTP, 10 mM phosphocreatine and 0.5% biocytin (pH 7.25, osmolarity 290 mOsm). Recordings in voltage clamp were performed by using the following intracellular solution (in mM): 117 cesium methansulfonate, 20 HEPES, 0.4 EGTA, 2.8 NaCl, 5 TEA-C1, 4 Mg-ATP, 0.3 Na-GTP, 10 QX314, 0.1 spermine and 0.5% biocytin (pH 7.3, osmolarity 290 mOsm). Access resistance was monitored throughout the duration of the experiment and data acquisition was suspended whenever the resting membrane potential was depolarized above −50 mV or the access resistance was beyond 20 MΩ. Recordings were amplified using up to two dual channel amplifiers (Multiclamp 700B, Molecular Devices), filtered at 2 kHz, digitized (20 kHz) and acquired using custom made software running on Igor Pro (Wavemetrics). Gabazine was obtained from Tocris.

Optogenetic stimulation was achieved through a 460-nm LED light source (XLED1, Lumen Dynamics) driven by TTL input with a delay onset of 25 μs (subtracted off-line). Light power on the sample was 33 mW mm⁻², and only the maximum power was employed. Slices were stimulated by single 2 ms light pulse repeated 20 times every 4 s or train of 15 light pulses at 10 Hz repeated 20 times every 6 s. In voltage-clamp cells were held at 0 mV for IPSC measurements, whereas, in current mode, EPSP and action potentials were measured at resting potentials.

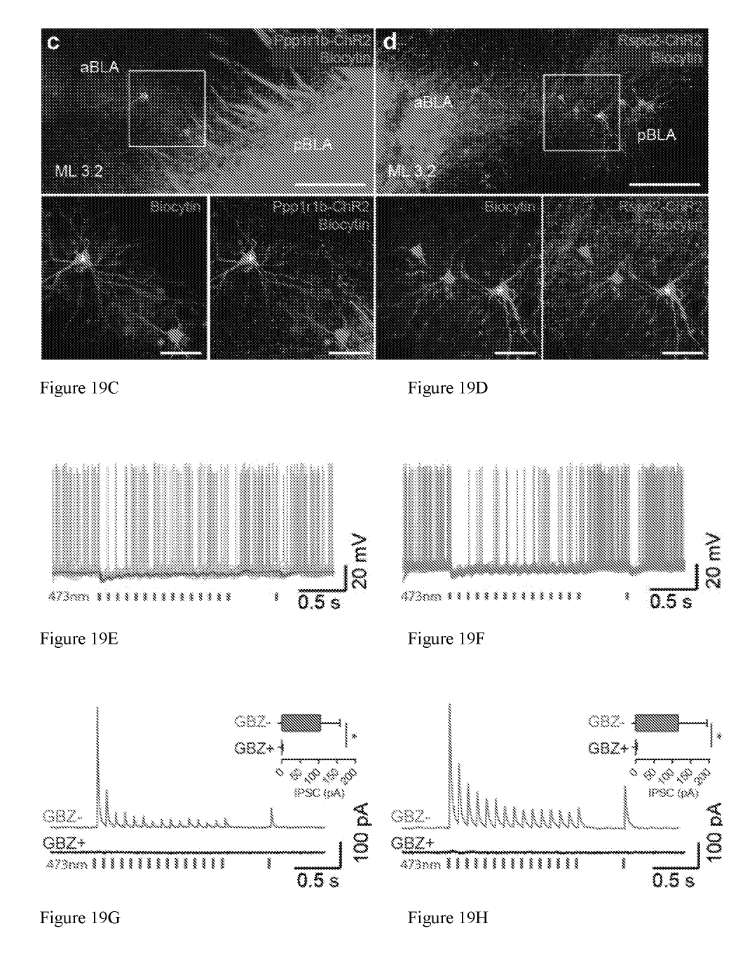

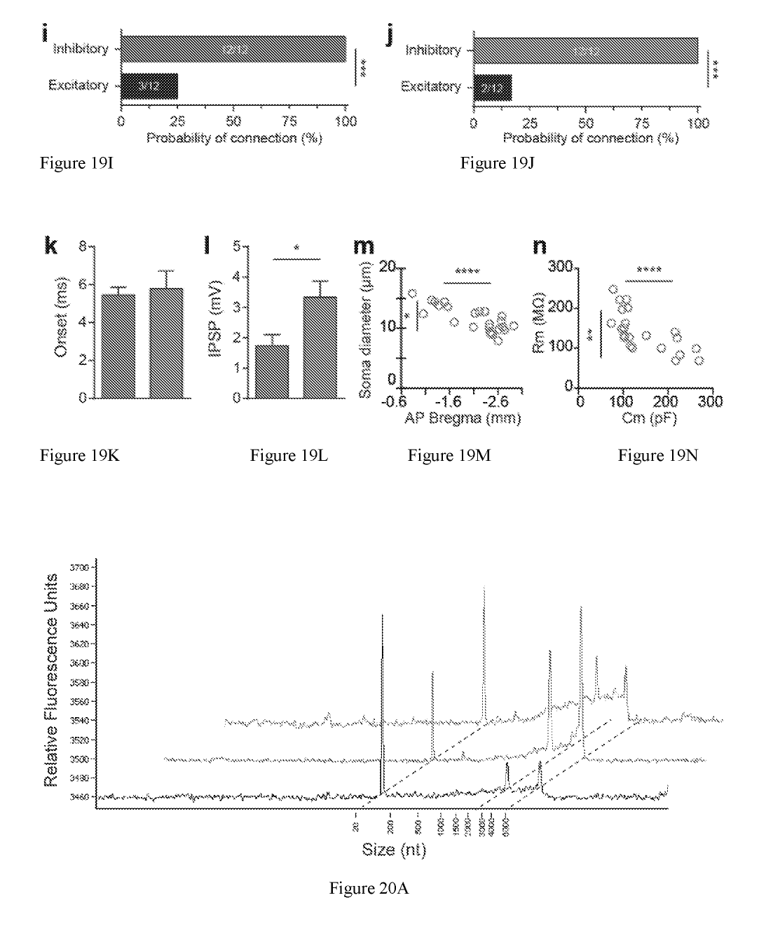

Morphological and electrophysiological criteria were established by using single cell RT-PCR to identify the molecular subtype. Magnocellular cells were identified based on location in the anterior part of the basolateral amygdala and by large soma size (13±0.5 m). Parvocellular cells were identified based on location in the posterior part of the basolateral amygdala, close to the ventral edge of the ventricle, and by small soma size (10±0.3 m). Physiological criteria such as membrane resistance and capacitance were employed to validate the cellular subtype (Table 2, FIGS. 19M, N).

The intrinsic electrophysiological properties were measured current mode with the cell held at −70 mV. Input resistance was estimated by linear fit of the I-V relationship (injection of 10-12 current steps of 1-s duration). Action potential threshold was tested with a current ramp injection. Membrane time constant was estimated by single exponential fit of the recovery-time from a −100 pA current step injection of 1-s duration. Synaptic connections, in voltage or current mode, were determined by averaging 20 trials. EPSC amplitude was measured from the average maximum peak response by subtracting a baseline obtained 5 ms before light pulse starts. EPSC onset was measured from the beginning of the light pulse to the starting point of the response estimated through the intercept between the baseline and a parabolic fit of the rising phase of the EPSC. To compute the probability of connection (n success/n tests) we employed only slices with reliable ChR2 expression characterized at least by one responsive postsynaptic cell (principal cell or interneuron).

Statistical analysis was performed using Igor (Wavemetrics), Graphpad (Prism), or Excel (Microsoft). The distribution of the data was tested with the Kolmogorov-Smirnov test and a two-tailed paired or unpaired t-test, or a Wilcoxon signed-rank or rank-sum test was employed accordingly. Fisher exact test was employed to verify the significance of the connection probability. Data are presented as mean±s.e.m.

Recorded slices were recovered for morphological identification as the recorded cells were filled with biocytin. Recorded slices were filled with biocytin and fixed in 4% paraformaldehyde for morphological identification.

### Single-Cell Quantitative Polymerase Chain Reaction.

In wild-type mouse brain slices, at the end of the patch clamp recordings, the cytoplasm of the recorded neuron was collected by applying negative pressure to the recording pipette. Once the cytoplasmic contents were suctioned, the glass pipette was quickly transferred to 0.2 mL PCR tube fill with 10 μL RNase-free water, 2 μL oligo(dT), 1 μL dNTP, 1 μL RNaseOUT provided by the SuperScript III CellsDirect cDNA Synthesis Kit (ThermoFisher). Samples were placed on a 70° C. heat block for 5 min, and then chilled on ice. For first strand synthesis, 8 μL of RT mix was added to the sample (6 μL 5×RT Buffer, 1 μL 0.1M DTT, 1 μL Superscript III RT) and incubated on a 50° C. heat block for 50 min. After the first strand synthesis, reverse transcriptase was inactivated by 5 min incubation on an 85° C. heat block. Samples were stored in −20° C. until quantitative polymerase chain reaction (qPCR).

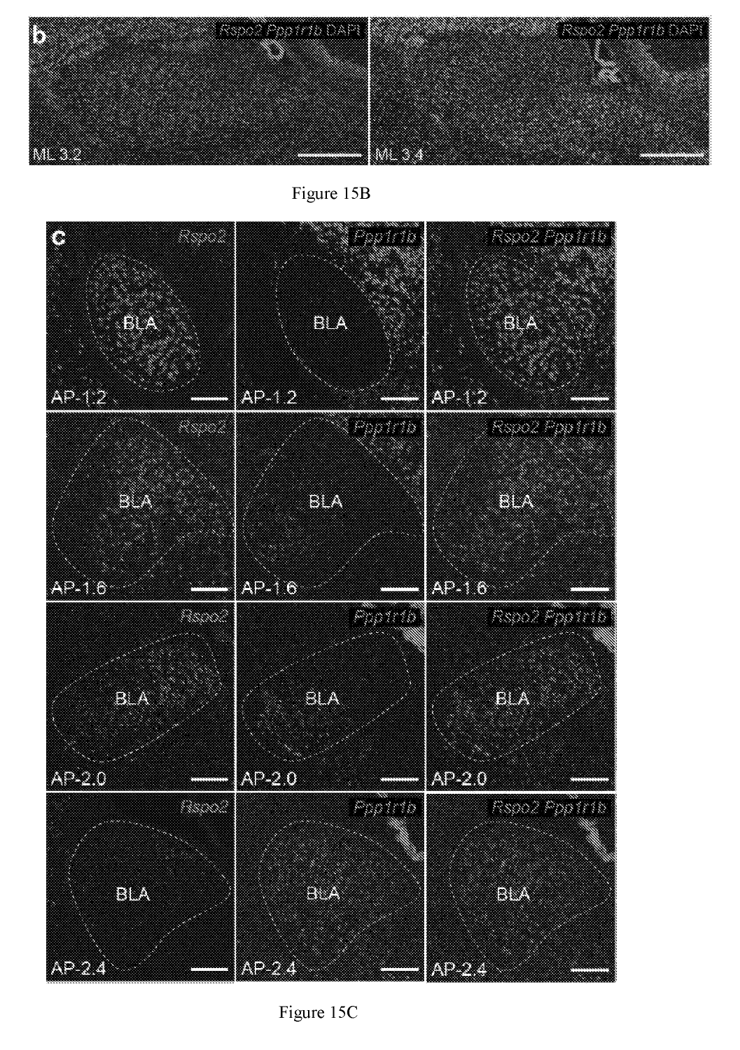

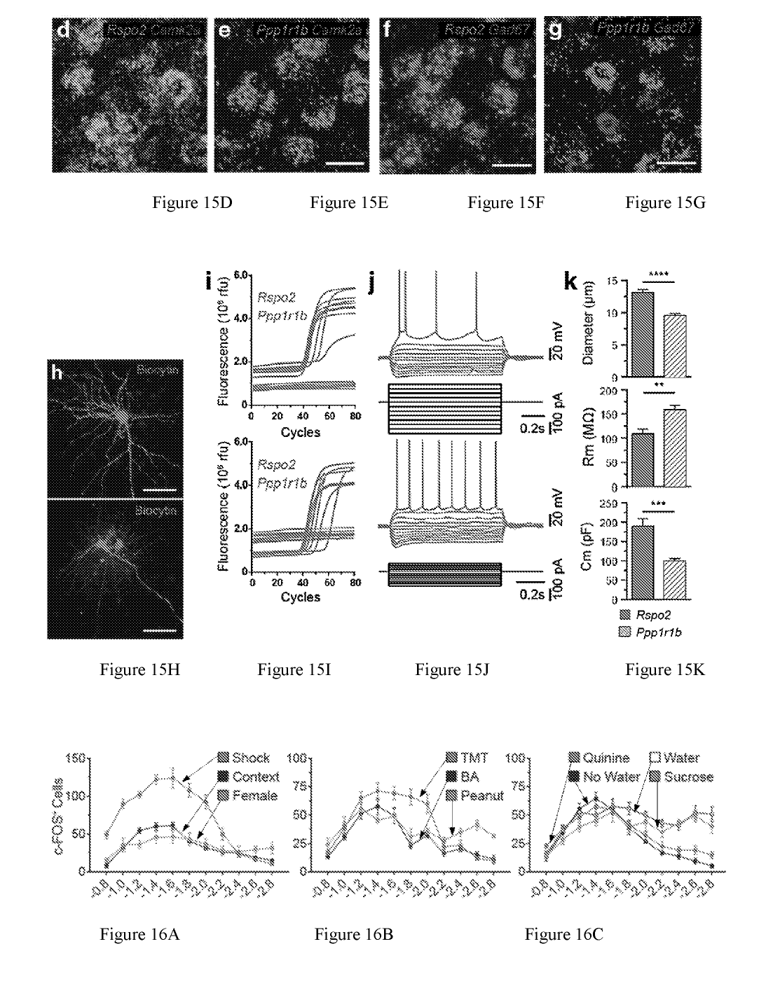

qPCR was performed using the Taqman Gene Expression Assays (Applied Biosystems). The genetic identity of BLA neuron using qPCR was determined by the ratio between Rpso2 and Ppplrlb expression. qPCR reaction consisted of 25 μL 2×TaqMan Gene Expression Master Mix, 2.5 μL of the 20×TaqMan Gene Expression Assay of Rspo2(Mm00555790_m1) or Ppplrlb (Mm00454892_m1), 7 μL of cDNA template, 17.5 μL of RNase free water. qPCR reaction was performed in an Applied Biosystems 7500 Real-Time PCR System using the Fluorescein (FAM) channel with the standard qPCR reaction protocol for 60-80 cycles. The majority of cells did not result in amplification of either Rspo2 or Ppplrlb. Therefore, Rpso2⁺ and Ppplrlb⁺ neurons were identified based on the criterion of any positive amplification. Rpso2⁺ or Ppplrlb⁺ amplification appeared at threshold cycles (CT)<50 cycle for most cells (FIG. 15I).

### Statistical Analysis.

Statistical analysis and statistical graphics was generated using GraphPad Prism 6.0. Sample sizes and statistical tests were determined based on previous studies examining similar behaviors and histology analyses. Variance was not significantly different between groups that were compared and met the assumptions of the statistical tests with the exception from groups where the effects of experimental manipulations were dramatic, such as in the case of Rspo2-ChR2 vs. Rspo2-EYFP in the optogenetic freeze test, or Ppplrlb-ChR2 vs. Ppplrlb-EYFP in the optogenetic self-stimulation test. All data are represented as mean±s.e.m.

### Results

### Identification of BLA Genetic Markers

Genetics-based RNA profiling strategies in mammalian models have involved ectopically expressing epitope-tagged RNA associated proteins or exploiting molecular modifications of RNA-associated substrates (53-56). In order to obtain transcriptional profiles, we implemented a strategy involving ectopically expressing an epitope-tagged RNA binding protein, poly(A) binding protein with a c-terminus FLAG tag (PABP-FLAG) (57). Two AAV9 constructs were used, one containing the tetracyclin-based transcription factor tTA under the control of the activity-dependent promoter of c-Fos (AAV9-c-Fos-tTA), and the other containing Pabp-flag under the control of the tetracycline response element TRE (AAV9-TRE-Pabp-flag). Activation of the c-Fos promoter drives the expression of tTA. In the absence of doxycycline (Dox), tTA binds TRE to induce the expression of PABP-FLAG. PABP-FLAG competes with endogenous PABP and bind the polyA tails of mRNA, which then can be isolated via immunoprecipiation using an antiflag antibody and A/G coated magnetic beads (FIG. 14A).

The putative negative and positive neurons were targeted by exposing male mice to shocks and a female mouse, respectively. AAV9-c-Fos-tTA and AAV9-TRE-Pabp-flag was introduced into the BLA in mice kept on a Dox diet. Once placed off a Dox diet for 2 days, mice were exposed to shocks or female mouse, then immediately placed back on a Dox diet for 2 days prior to sacrifice. A similar number of BLA neurons were FLAG+ in the shock and female groups, but were greater than mice that were kept in their home cages or kept on a Dox diet (FIGS. 14C,B,D,E,G, J). In contrast, a greater number of BLA neurons were FLAG+ in the mice that underwent kainic acid-induced seizures compare to the shock or female group (FIGS. 14B,F-J). This affirms the activity-dependency of the genetic system. Therefore, RNA immunoprecipitation using antibodies against FLAG was performed from the shock and female group. Isolated RNA was reverse-transcribed to cDNA and underwent microarray analysis using Affymetric Mouse 430A chip. After RMA or MAS5 normalization (see Methods), differential gene expression profiles were compared between the shock and female group and were used as the basis of the screen for identifying genetic markers for the putative negative and positive neurons of the BLA (FIG. 14K, FIG. 20).

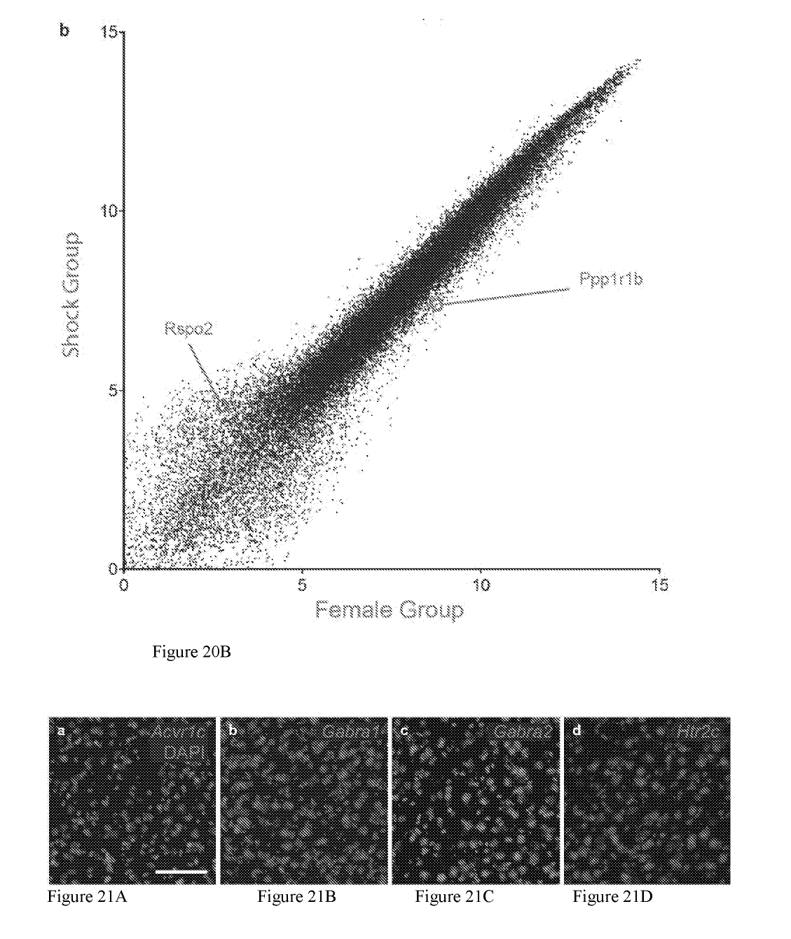

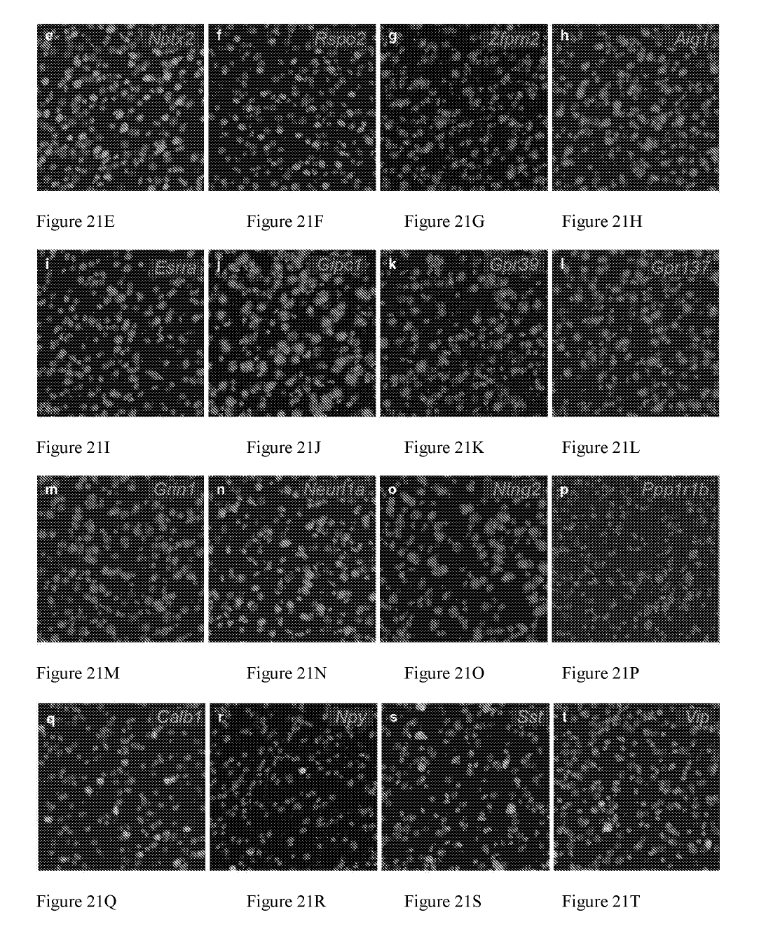

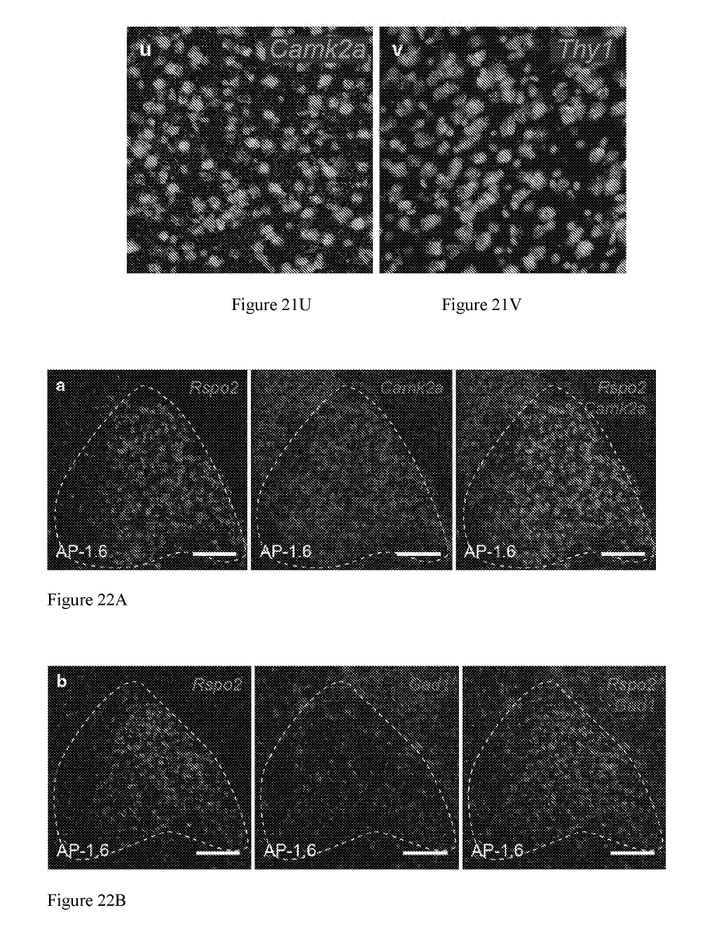

Independent of statistical significance, hundreds of genes that were the most enriched in the shock and female groups were individually screened on Allen Mouse Brain Atlas (5). 37 genes were selected for single label fluorescent in situ hybridization, of which, 16 probes yielded a quantifiable signals in the −1.0 to −1.6 anterior-posterior (AP) plane of the BLA (FIG. 21). Quantification of gene expression in the BLA revealed that the majority of candidate genes were expressed in a virtually all BLA principle neurons (FIG. 14L, FIG. 21). Rspondin-2 (Rspo2) and Protein phosphatase 1 regulatory to subunit 1B (Ppplrlb) (also known as DARPP-3222) were expressed in a subpopulation of BLA neurons and selected for further characterization (FIG. 14L).

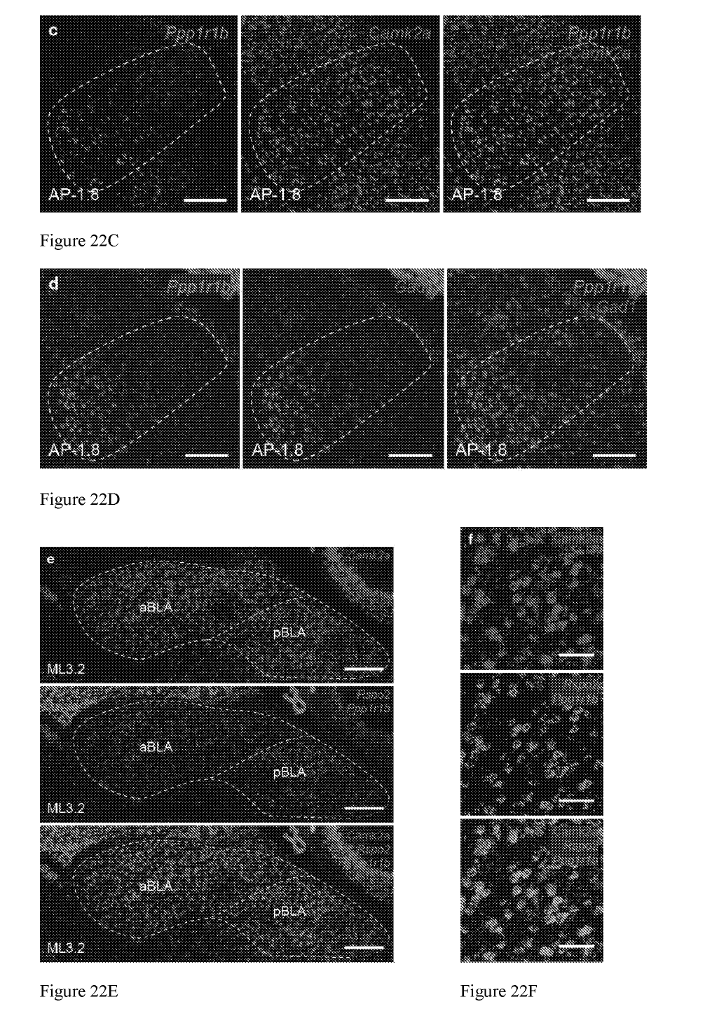

Double label single molecule fluorescent in situ hybridization (smFISH) and quantification across the anterior-posterior (AP) axis of the BLA (−0.8 to −2.8 mm from bregma) revealed that Rspo2 and Ppplrlb labeled spatially segregated population of neurons (FIG. 15A-C). Less than 1% of BLA neurons were Rspo2⁺Ppplrlb⁺ (Table 1). Rspo2⁺ and Ppplrlb⁺ BLA neurons are co-labelled with the pyramidal neurons marker, Camk2a, and non-overlapping with the inhibitory neuron marker, Gad1 (FIG. 15D-G, Table 1). Rspo2⁺ neurons correspond to magnocellular pyramidal neurons in the anterior BLA (aBLA). In contrast, Ppplrlb⁺ neurons correspond to the parvocellular pyramidal neurons or posterior BLA (pBLA) (38,60). Double smFISH with a Camk2a probe and the combined probes of both the Rspo2 and Ppplrlb showed that virtually all Camk2a⁺ BLA neurons express either Rspo2 or Ppplrlb (FIG. 22). Therefore, Rpso2⁺ and Ppplrlb⁺ neurons collectively define the entirety of BLA pyramidal neurons.

TABLE 1
Anatomical and Genetic Characterization of BLA Neurons.
Anatomical and genetic characterization of BLA neurons

| | Rspo2⁺ | Ppp1r1b⁺ | Rspo2⁺ Ppp1r1b⁺ |
|---|---|---|---|
| Total Neurons (n = 3) | 3611 | 2311 | 54 |
| Mean Proportion (%) | 59.9 ± 1.28 | 39.1 ± 1.14 | 0.970 ± 0.191 |
| | Rspo2⁺ | Gad1⁺ | Rspo2⁺ Gad1⁺ |
| Total Neurons (n = 1) | 303 | 112 | 0 |
| | Ppp1r1b⁺ | Gad1+ | Ppp1r1b⁺ Gad1⁺ |
| Total Neurons (n = 1) | 190 | 116 | 0 |
| | (Rspo2 + Ppp1r1b)⁺Camk2⁺ | (Rspo2 + Ppp1r1b)⁺Camk2⁺ | (Rspo2 + Ppp1r1b)⁺Camk2⁺ |
| Total Neurons (n = 4) | 2361 | 0 | 0 |
| | CTB-CeC⁺Rspo2⁺ | CTB-CeC Ppp1r1b⁺ | |
| Total Neurons (n = 3) | 423 | 16 | |
| Mean Proportion (%) | 96.2 ± 0.945 | 3.78 ± 0.945 | |
| | CTB-CeL/M⁺Rspo2⁺ | CTB-CeL/M⁺Ppp1r1b⁺ | |
| Total Neurons (n = 3) | 64 | 1012 | |
| Mean Proportion (%) | 5.58 ± 1.74 | 94.4 ± 1.74 | |
| | CTB-Nac⁺Rspo2⁺ | CTB-NAc⁺Ppp1r1b⁺ | |
| Total Neurons (n = 3) | 344 | 775 | |
| Mean Proportion (%) | 30.7 ± 3.53 | 69.2 ± 3.53 | |

The electrophysiological and morphological properties of Rspo2⁺ and Ppplrlb⁺ neurons were examined using patch clamp recordings. Rspo2⁺ and Ppplrlb⁺ were targeted by patching magnocellular and parvocellular BLA neurons (FIG. 15H). To be certain on the genetic identity, Rspo2⁺ and Ppplrlb⁺ neurons were identified by the use of single-cell quantitative polymerase chain reaction (qPCR) from cytoplasmic harvest of patch clamped recorded BLA neurons. Of 37 magnocellular neurons, single cell qPCR yielded 10 Rpso2⁺ and 0 Ppplrlb⁺ neurons; of 38 parvocellular neurons, single cell qPCR yielded 0 Rpso2⁺ and II Ppplrlb⁺ neurons (FIG. 15I). Soma diameter was larger in Rspo2⁺ neurons than Ppplrlb⁺ neurons; membrane resistance was smaller in Rspo2⁺ neurons than Ppplrlb⁺ neurons; membrane capacitance was larger in Rspo2⁺ neurons than Ppplrlb⁺ neurons (FIGS. 15J,K). qPCR-confirmed Rspo2⁺ and Ppplrlb⁺ neurons were not significantly different from unconfirmed magnocellular and parvocellular neurons, respectively (Table 2). Taken together, Rpso2⁺ and Ppplrlb⁺ BLA neurons defined spatially segregated, genetically, morphologically, and electrophysiological distinct cell-types.

TABLE 2
Morphological and Physiological characterization of BLA Neurons.
Morphological and physiological characterization of BLA neurons

| | Soma diameter (μm) | Vm (mV) | Rm (MΩ) | Cm (pF) | Spike threshold (mV) | Rheobase (pÅ) |
|---|---|---|---|---|---|---|
| Cell type | | | | | | |
| Magnocellular (n = 37) | 12.8 ± 0.2 | −60.9 ± 0.9 | 103.1 ± 4.7 | 197.6 ± 10 | −37.7 ± 0.4 | 213.7 ± 10.5 |
| Parvocellular (n = 38) | 9.4 ± 0.2 | −55.6 ± 0.8 | 165.5 ± 6.5 | 102.2 ± 4.1 | −35.5 ± 0.5 | 137.7 ± 6.7 |
| Rspo2⁺ (n = 10) | 13.1 ± 0.5 | −62.5 ± 2.1 | 108.9 ± 9.6 | 190.3 ± 19.1 | −37.6 ± 0.9 | 198.7 ± 18 |
| Ppp1r1b⁺ (n = 11) | 9.5 ± 0.3 | −57.1 ± 1.6 | 158.7 ± 9.5 | 99.5 ± 5.9 | −34.8 ± 1.1 | 152.3 ± 15.2 |
| Unpaired t-test P | | | | | | |
| Magno. (n = 37) vs | 7.00E−17 | 0.00003 | 6.00E−11 | 1.00E−11 | 0.0008 | 8.00E−08 |
| Parvo. (n = 38) | | | | | | |
| Rapa2⁺ (n = 10) vs | 0.00002 | 0.051 | 0.0016 | 0.0009 | 0.06 | 0.06 |
| Ppp1r1b⁺ (n = 11) | | | | | | |
| Magno. (n = 27) vs | 0.5 | 0.4 | 0.5 | 0.7 | 0.9 | 0.4 |
| Rspo2⁺ (n = 10) | | | | | | |
| Parvo. (n = 27) vs | 0.6 | 0.3 | 0.5 | 0.6 | 0.5 | 0.2 |
| Ppp1r1b⁺ (n = 11) | | | | | | |

### BLA Activation by Valence-Specific Stimuli

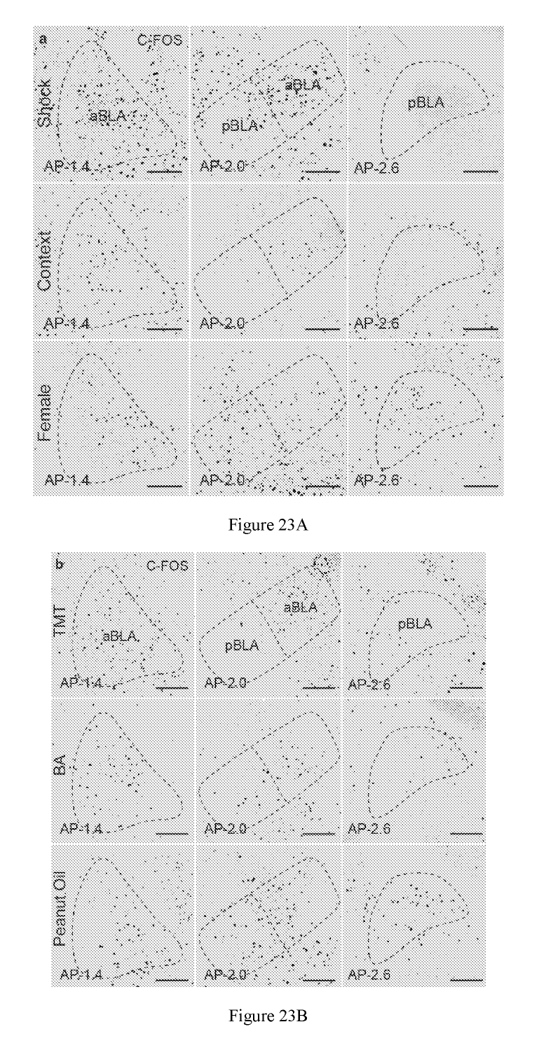

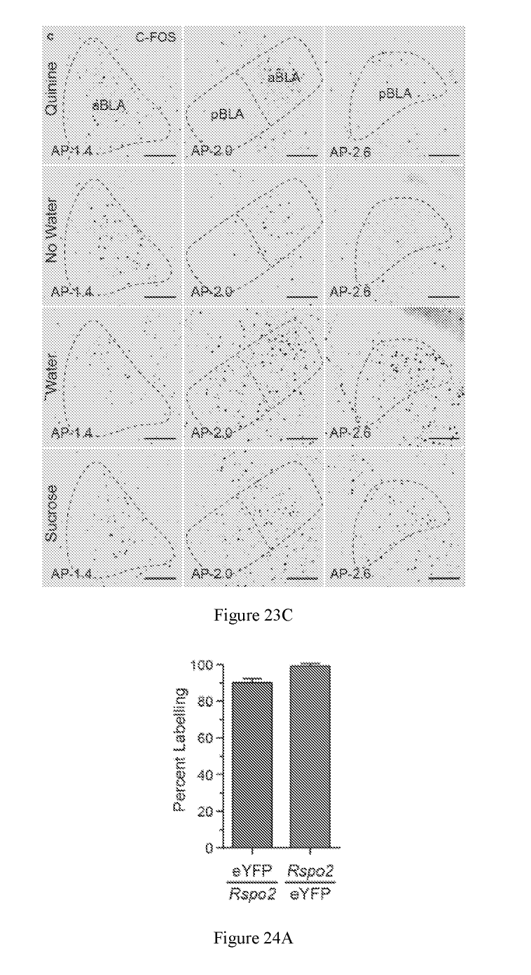

If Rspo2⁺ and Ppplrlb⁺ neurons represent negative and positive neurons of the BLA, respectively, then valence may be encoded along the AP axis of the BLA as a reflection of Rspo2 or Ppplrlb expression. Mice were exposed to the stimuli used to identify BLA gene markers—shocks or female mice—end were sacrificed 90 minutes later. The distribution of c-FOS⁺ neurons was quantified in the BLA by measuring the total number of c-FOS⁺ cells per section at intervals across the AP axis (FIG. 16A-C, FIG. 23). The relative c-FOS expression, measured by the number of c-FOS⁺ neurons as a percentage of total c-FOS⁺ BLA neurons, was significantly greater in the aBLA in response to shocks compared to exposure to a female mice or control mice, which is received no stimulus in a context (FIG. 16D). Conversely, relative c-FOS expression was significantly greater in the pBLA in response to female mice compared to exposure to shock or control, which were exposed to a neutral context (FIG. 16D). In response to valence-specific olfactory stimuli—2,3,5-Trimethyl-3-thiazoline (TMT), or peanut oil—relative c-FOS expression was significantly greater in the aBLA in response to TMT compared to exposure to a neutral odor benzaldehyde (BA) or peanut oil, while relative c-FOS expression was significantly greater in the pBLA in response to peanut oil compared to exposure to a BA or TMT (FIG. 16E). In response to valence-specific gustatory stimuli—quinine (bitter), water, sucrose (sweet)—relative c-FOS expression was significantly greater in the pBLA in response to water and sucrose water compared to mice that received no water or quinine water (FIG. 16F). In contrast, no significant difference was observed in relative c-FOS expression between exposure to quinine water (which did not elicit much water drinking) compared to no water, as well as between sucrose water and water (FIG. 16F). Overall, the aBLA is recruited by stimuli that elicits negative behaviors (shocks, TMT), while the pBLA is recruited by stimuli that elicits positive behaviors (female, water, sucrose, peanut oil).

Double smFISH was performed to directly assess the expression of c-Fos in Rspo2⁺ or Ppplrlb⁺ BLA neurons in response to valence-specific stimuli. Shocks significantly increases c-fos expression in Rspo2⁺ (FIGS. 16G,K), but not in Ppplrlb⁺ neurons (FIGS. 16H,L), compared to context (FIGS. 16G, H, M, N). In contrast, administration of water significantly increases c-Fos expression in Ppplrlb⁺ (FIGS. 16J,P), but not Rspo2⁺ neurons (FIGS. 16I,O), compared no water (FIGS. 16I, J, Q, R). These data suggest that negative and positive information is represented by genetically-defined populations of neurons in the BLA that are spatially segregated; Rspo2⁺ neurons, which define the aBLA, represent negative valence, while Ppplrlb+ neurons, which define the pBLA, represent positive valence.

### BLA in Valence-Specific Behaviors

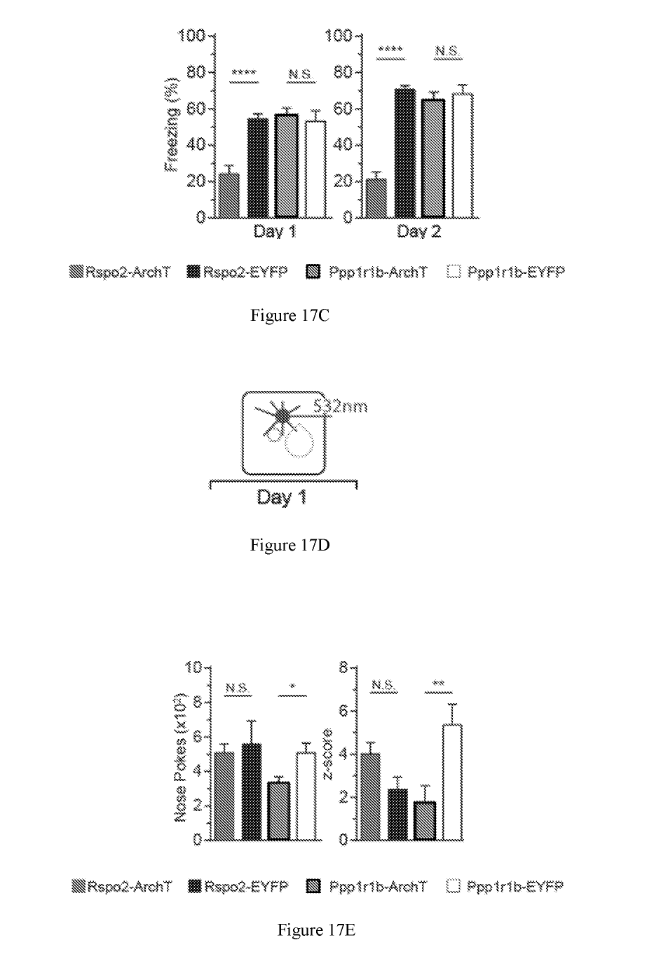

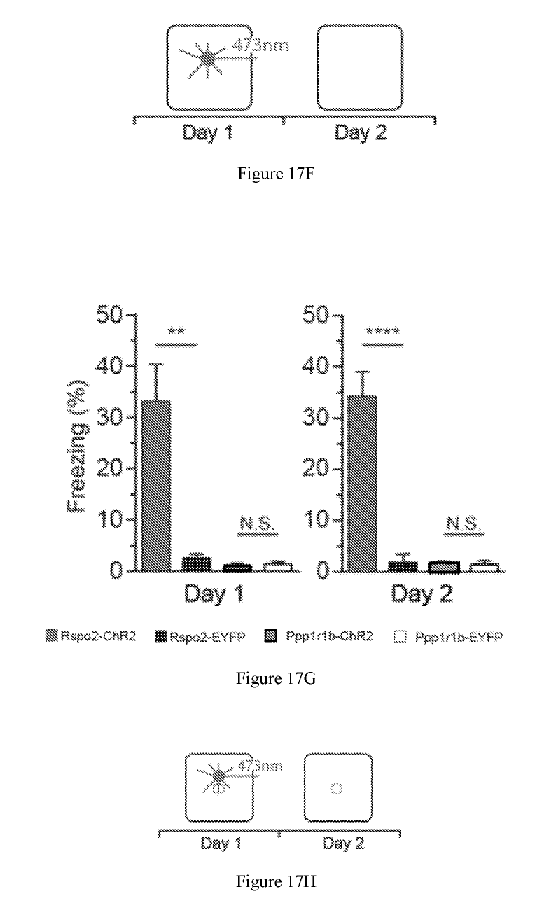

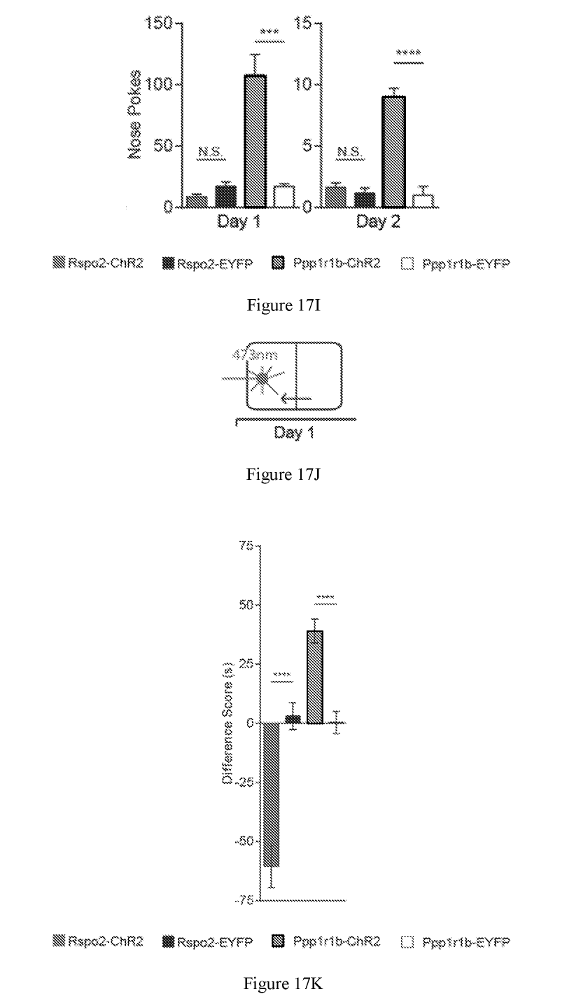

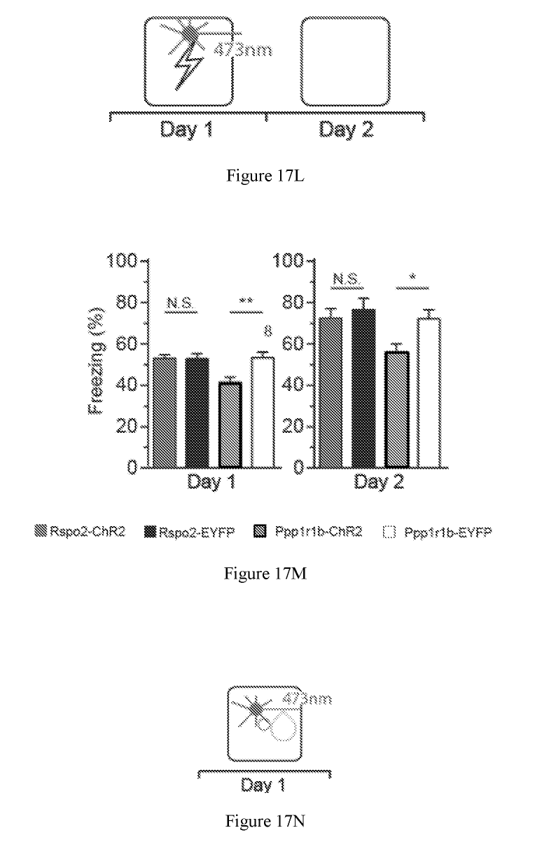

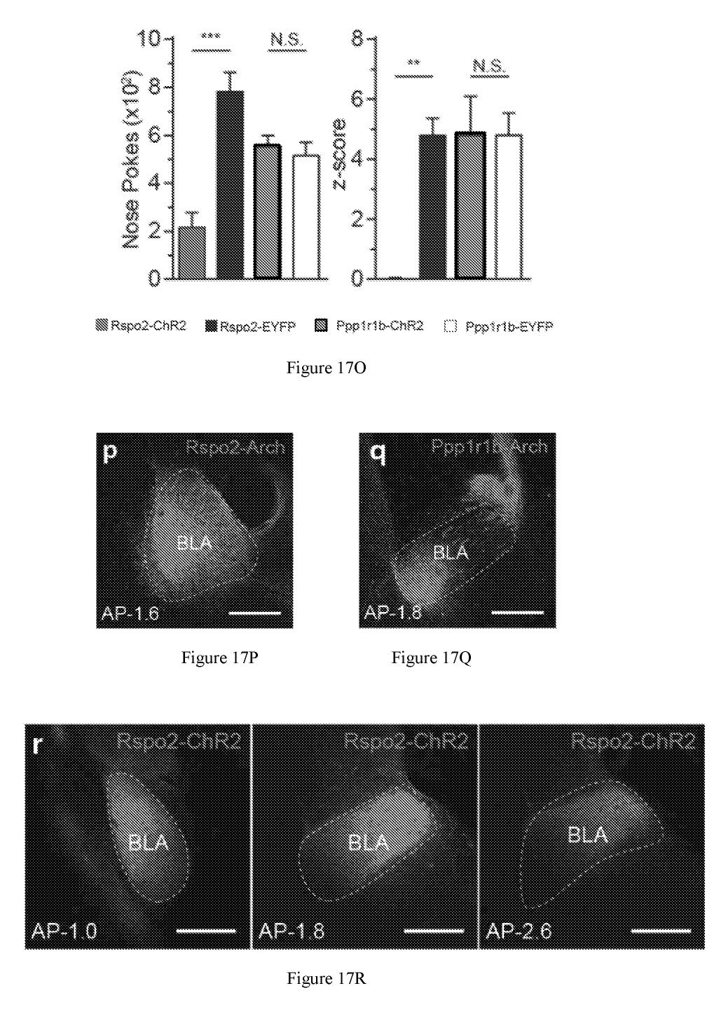

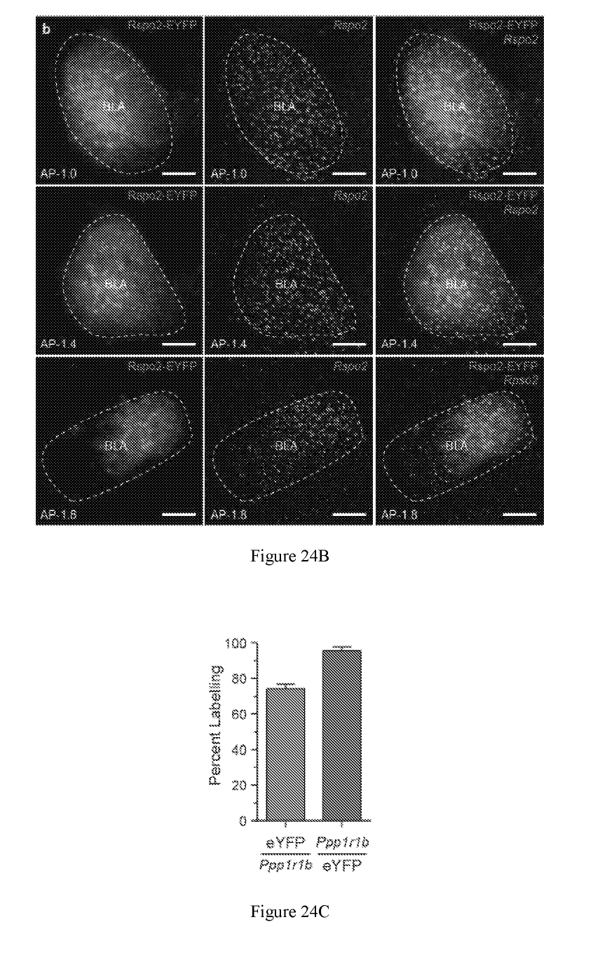

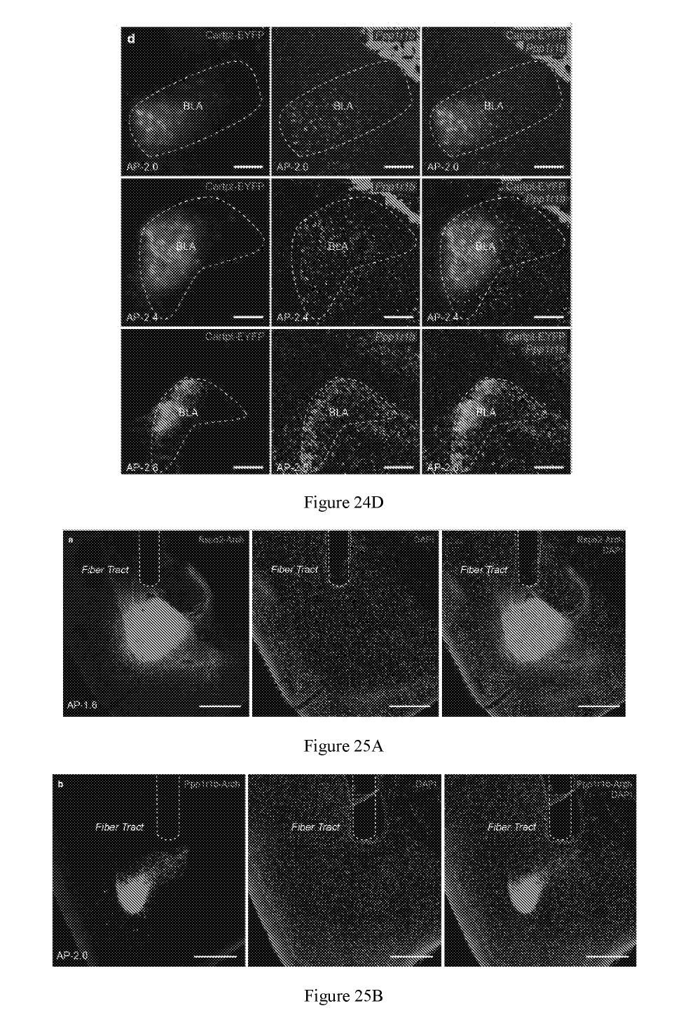

Valence-specific activation of Rpso2⁺ and Ppplrlb⁺ neurons posits that these populations may be necessary for valence-specific behaviors; therefore, the effects of inhibiting these BLA populations were performed in a fear and reward conditioning paradigm. Rspo2⁺ and Ppplrlb⁺ neurons were genetically targeted using Rspo2-Cre and Cartpt-Cre mice, respectively (FIG. 24). Ppplrlb⁺ BLA neurons are accessible by Cartpt-Cre mice, and hereafter, virus-injected Cartpt-Cre mice will be referred to using “Ppplrlb”. Light-activated inhibitory ion channel, eArch3.0, was expressed in Rspo2⁺ (Rspo2-Arch) and Ppplrlb⁺ (Ppplrlb-Arch) BLA neurons using a Cre-dependent viral vector (AAV5-EF1α-DIO-eArch3.0-eYFP) bilaterally targeted to the BLA of Rspo2-Cre and Cartpt-Cre mice, respectively. Control mice (Rspo2-eYFP, Ppplrlb-eYFP) received a viral vector lacking eArch3.0, (AAV5-EF1α-DIO-eYFP) (FIGS. 17A,P,Q, FIG. 25).

On day 1 of contextual fear conditioning, mice received green light, bilaterally targeted to the BLA, during shocks (FIG. 17B). Rspo2-Arch mice displayed reduced levels of freezing in response to shocks compared with Rspo2-eYFP mice. Ppplrlb-Arch mice displayed similar levels of freezing compared to Ppplrlb-eYFP mice. On day 2, mice were tested in the context without shock or light stimulation. Reduction of freezing was observed in Rspo2-Arch mice compared to Rspo2-GFP mice, while, similar levels of freezing was observed in Ppplrlb-Arch mice compared to Ppplrlb-eYFP mice (FIG. 17C). Thus, Rspo2⁺, but not Ppplrlbp⁺, BLA neuronal activity is necessary for freezing to shock stimuli and for the association of a context to freezing behavior.

Reward conditioning took place in an operant conditioning chamber, where water was dispensed contingent on a nose poke following an external light cue (FIG. 17D). Green light was bilaterally delivered into the BLA simultaneously with the presentation of water. Rspo2-Arch and Rspo2-eYFP mice displayed similar levels of nose pokes and cue-reward association (z-score of time spent in the reward port during cue period). In contrast, Ppplrlb-Arch mice displayed reduced levels of nose pokes and cue-reward association compared to Ppplrlb-eYFP mice (FIG. 17E). Thus, Ppplrlb⁺, but not Rpso2⁺, BLA neuronal activity is necessary for reward-seeking behavior and for the association of a conditioned stimulus to appetitive behavior.

Next, the effects of activating these BLA neurons were assessed. Light-activated excitatory ion channel, ChR2, was expressed in Rspo2⁺ (Rspo2-ChR2) and Ppplrlb⁺ (Ppplrlb-ChR2) BLA neurons using a Cre-dependent viral vector (AAV5-EF1α-DIO-ChR2-eYFP) unilaterally targeted to the BLA of Rspo2-Cre and Cartpt-Cre mice, respectively. Control mice (Rspo2-eYFP, Ppplrlb-eYFP) received a viral vector lacking ChR2, (AAV5-EF1α-DIO-eYFP) (FIGS. 17A,R,S).

On day 1 of the optogenetic freezing test, mice were placed in a neutral context while receiving blue light stimulation (FIG. 17F). Rspo2-ChR2 mice displayed greater levels of freezing compared to Rspo2-eYFP mice, while Ppplr1rb-ChR2 and Ppplrlb-eYFP mice displayed similar levels of freezing (FIG. 17G). On day 2, mice were returned to the context and freezing was measured without shock. Rspo2-ChR2 mice displayed greater levels of freezing compared to Rspo2-eYFP mice, while Ppplr1rb-ChR2 and Ppplrlb-eYFP mice displayed similar levels of freezing (FIG. 17G). Thus, Rspo2⁺, but not Ppplrlb⁺, BLA neurons are sufficient to elicit freezing, which can be conditioned to a neutral context.

On day 1 of the optogenetic self-stimulation test, mice were placed in an operant conditioning chamber in which blue light stimulation was administered when poking into a nose port (FIG. 17H). Ppplrlb-ChR2 mice displayed greater number of pokes compared to Ppplrlb-eYFP mice, while Rspo2-ChR2 and Rpso2-eYFP mice displayed similar number of pokes. On day 2, mice were returned to the operant condition chamber in which no light stimulation was delivered. Ppplrlb-ChR2 mice displayed greater number of pokes compared to Ppplrlb-eYFP mice, while Rspo2-ChR2 and Rspo2-eYFP mice displayed similar number of pokes (FIG. 17I). Thus, Ppplrlb⁺, but not Rspo2⁺, BLA neurons are sufficient to elicit self-stimulation and support reward conditioning.

In real-time optogenetic place preference test (FIG. 17J), Rspo2-ChR2 mice spent less time in the light-stimulated side compared to corresponding controls, while Ppplrlb-ChR2 mice spent more time in the light-stimulated side compared to corresponding controls (FIG. 17K). Therefore, Rspo2⁺ BLA neurons are sufficient to elicit place aversion while Ppplrlb⁺ BLA neurons are sufficient to elicit place preference.

### Antagonism of Valence-Specific Behaviors

Rpso2⁺ and Ppplrlb⁺ neurons drive opposing behaviors; therefore, the effects of optogenetically activating Rpso2⁺ and Ppplrlb⁺ neurons during the presence of valence-specific stimuli was examined. On day 1 of contextual fear conditioning, ChR2-expressing mice received bilateral blue light stimulation during shocks (FIG. 17L). Rspo2-ChR2 and Rspo2-eYFP mice displayed similar levels of freezing in response to shocks. In contrast, Ppplrlb-ChR2 mice displayed lower levels of freezing than Ppplrlb-eYFP mice. On day 2, conditioned responses of freezing were assessed by exposing mice to the conditioned context without shock or light stimulation. Similar to day 1, no difference in freezing was observed between Rspo2-ChR2 and Rspo2-eYFP mice, while less freezing was observed in Ppplrlb-ChR2 mice compared Ppplrlb-eYFP mice (FIG. 17M). Thus, activation of Ppplrlb⁺ BLA neurons is sufficient to disrupt freezing to shocks and the association of a contextual conditioned stimulus with freezing.

In reward conditioning, ChR2-expressing mice received blue light stimulation during reward delivery (FIG. 17N). Rspo2-ChR2 displayed reduced levels of nose pokes and cue-reward association compared to Rspo2-eYFP mice. Ppplrlb-ChR2 and Ppplrlbp-eYFP mice displayed similar levels of nose pokes and cue-reward association (FIG. 17O). Thus, activation of Rspo2⁺ BLA neurons is sufficient to disrupt reward-seeking behaviors and the association of an appetitive behavior to a conditioned stimulus.

### Negative and Positive Circuit of the BLA

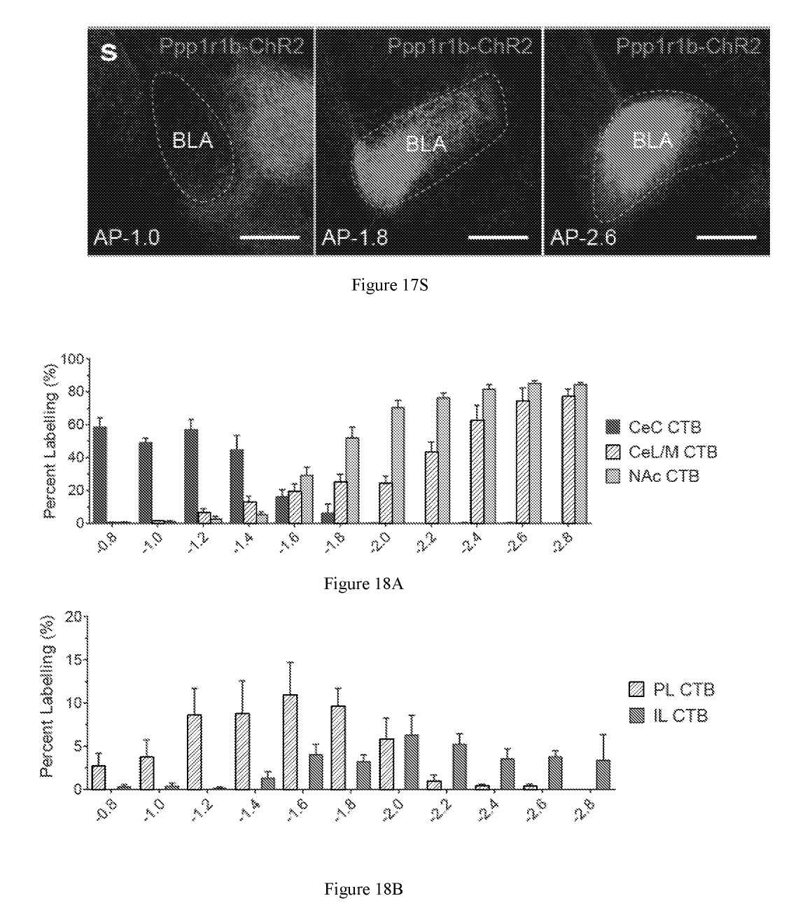

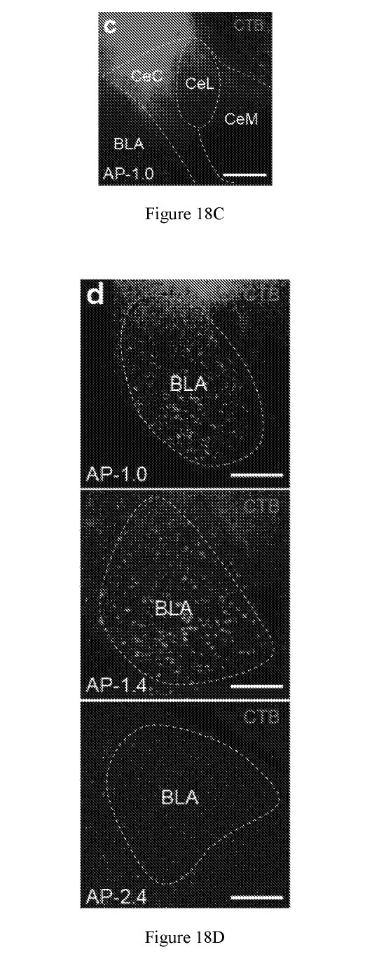

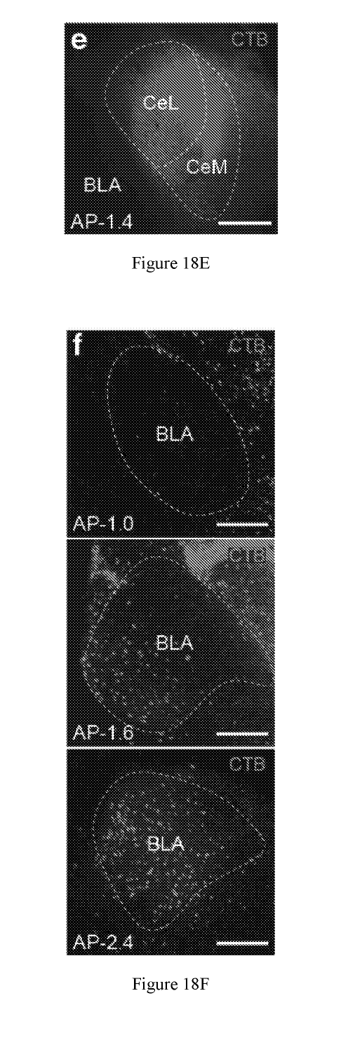

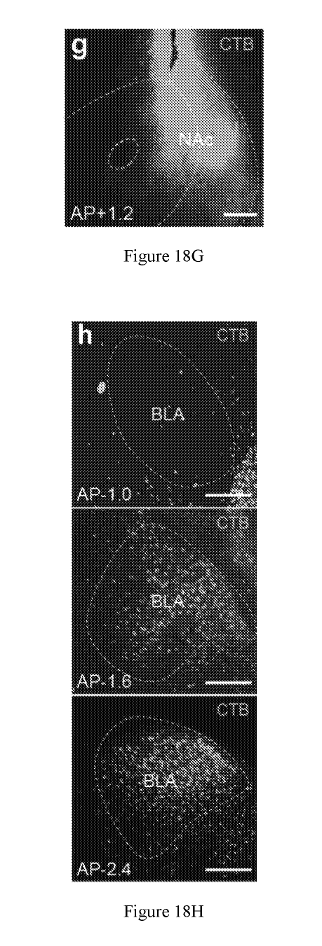

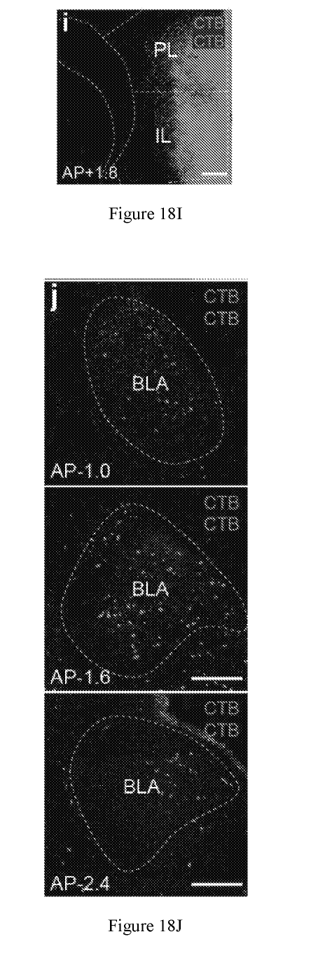

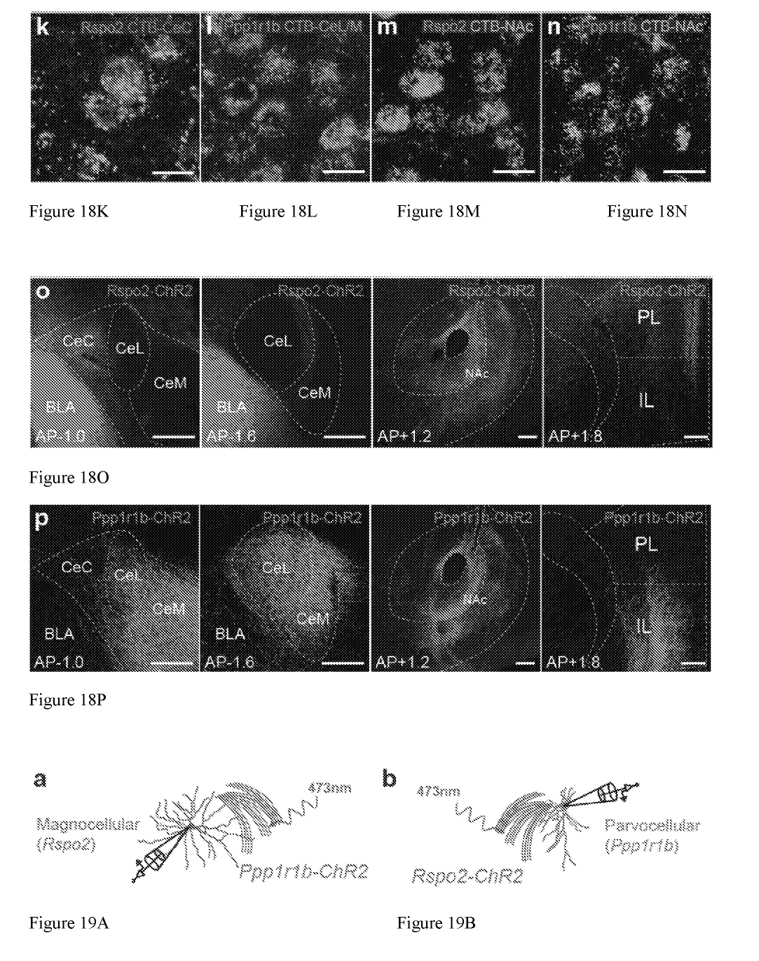

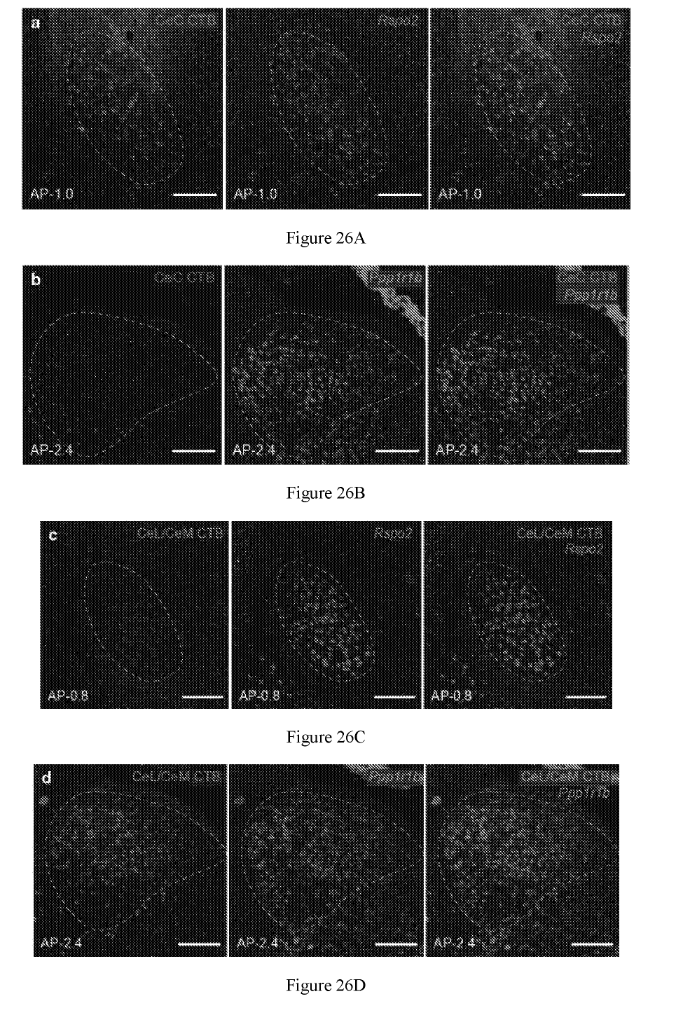

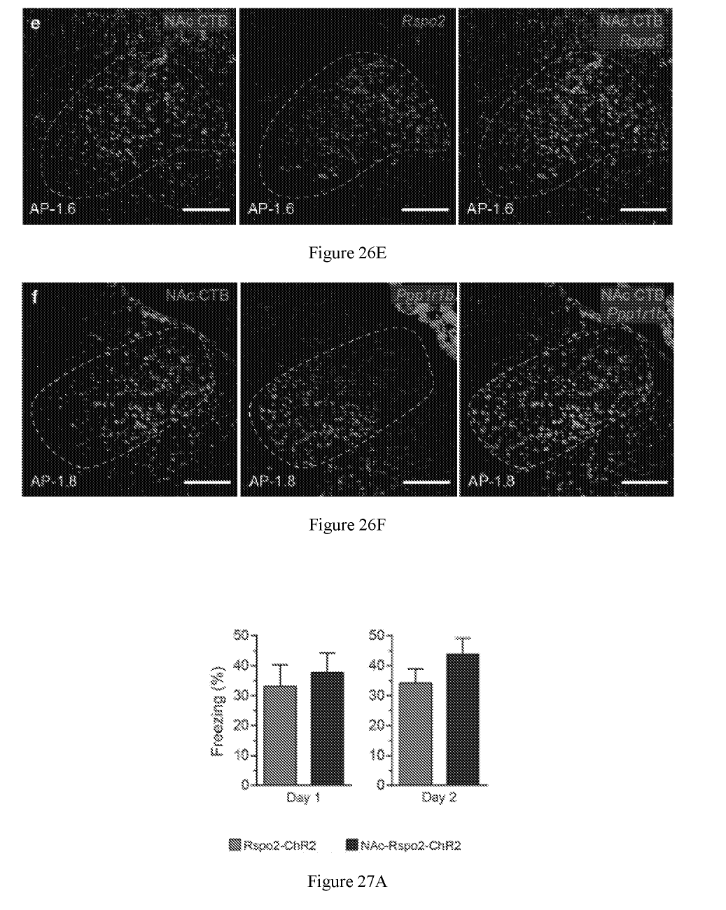

The distinct projection targets of the Rspo2⁺ and Ppplrlb⁺ neurons may reveal divergent brain structure that mediate negative and positive behaviors. Therefore, retrograde tracing from putative projection targets was examined using cholera toxin subunit b (CTB). CTB targeted to the capsular nucleus of the central amygdala (CeC), revealed CTB⁺ neurons primarily in the aBLA (FIGS. 18A,C,D). CTB targeted to the lateral/medial nucleus of the central amygdala (CeL/CeM), resulted in CTB⁺ neurons distributed along the lateral side of the pBLA (FIGS. 18A,E,F). CTB targeted to the nucleus accumbens (NAc), resulted in CTB⁺ neurons distributed along the medial side of the BLA, spanning the posterior end of the aBLA to the posterior end of the pBLA (FIG. 18A,G,H). Dual-labelled CTB targeted to the prelimbic (PL) and inframlimbic (IL) cortex resulted in spatially segregated distribution of CTB⁺ neurons in the BLA-PL-CTB⁺ neurons primarily in the aBLA, IL-CTB⁺ neurons primarily in the pBLA (FIGS. 18B,I,J). smFISH of Rspo2 or Ppplrlb⁺ probe in CTB injected mice, revealed that CeC-CTB⁺ BLA neurons are 96% Rpso2⁺ and 4% Ppplrlb⁺; CeL/CeM-CTB neurons are 6% Rspo2⁺ and 94% Ppplrlb⁺; NAc CTB+ neurons are 30% Rpso2⁺ and 70% Ppplrlb⁺ (FIG. 18K-N, FIG. 26, Table 1).

Anterograde characterization of ChR2-eYFP⁺ fibers in Rspo2-ChR2 and Ppplrlb-ChR2 mice was examined. In Rspo2-ChR2 mice, dense fibers were found in the CeC, NAc, PL, but not in the CeL, CeM, or IL (FIG. 18O). In Ppplrlb-ChR2 mice, dense fibers were found in the CeL, CeM, NAc, and IL but not in the CeC or PL (FIG. 18P). Together, from CTB retrograde tracing and anterograde characterization of projection fibers suggest that Rspo2⁺ distinctly project to the CeC and PL, Ppplrlb⁺: neurons distinctly project to the CeL, CeM, and IL, while Rspo2 and Ppplrlb⁺ BLA neurons both project to the NAc.

Anatomical and functional relationship between Rspo2⁺ and Ppplrlb⁺ BLA neurons was examined in order to identify a circuit mechanism underlying behavioral antagonism. The functional relationship between Rspo2⁺ and Ppplrlb⁺ neurons were examined by combining patch clamp recordings with optogenetic stimulation of cell type-specific axons (FIG. 19A-D). Patch clamp recordings of Rspo2⁺ and Ppplrlb⁺ neurons revealed distinct intrinsic physiological properties (Table 2). Therefore, the postsynaptic cell target was recognized based on a combination of anatomical position, soma size, and intrinsic electrophysiological properties (FIGS. 19M,N). Electrophysiological recordings of magnocellular cells in response to optogenetic stimulation of Ppplrlb-ChR2⁺ fibers and recordings of parvocellular cells in response to stimulation of Rspo2-ChR2⁺ fibers resulted in inhibitory post-synaptic potentials (IPSPs) (FIG. 19E-H,K,L). The probability of connections of magnocellular to parvocellular BLA neurons and vice versa were 100% and 100% inhibitory (FIGS. 19I,J). 25% of connections of parvocellular to magnocellular BLA neurons and 17% of connections of magnocellular to parvocellular were both inhibitory and excitatory (FIGS. 19I,J). These data suggest that these two populations interact predominantly through mutual inhibition.

### DISCUSSION

A forward genetic strategy has now been demonstrated and used to transcriptionally profile active neurons in BLA. This approach revealed genetic markers for distinct populations of BLA neurons and was predictive of neuronal function. Rspo2⁺ BLA neurons are activated by stimuli that elicit negative behaviors, while Ppplrlb⁺ BLA neurons are activated by stimuli that elicit positive behaviors. Rspo2⁺ BLA neurons are necessary and sufficient for negative behaviors and associations, while Ppplrlb⁺ BLA neurons are necessary and sufficient for positive behaviors and associations. Rspo2⁺ and Ppplrlb⁺ neurons antagonize valence-specific behaviors and make reciprocal inhibitory connections. Collectively, these results support a model in which mutually inhibitory Rspo2⁺ and Ppplrlb⁺ neurons are the principle neurons that represent and elicit negative and positive behaviors, respectively.

Anatomically, Rspo2⁺ BLA neurons correspond to the magnocellular pyramidal cells of the aBLA, while Ppplrlb⁺ BLA neurons correspond to the parvocellular pyramidal cells of the pBLA. Previous inactivation studies have implicated a greater contribution of the aBLA in contextual fear conditioning (47), and the pBLA in reward conditioning (61). Here, it was established, using specific genetic markers for cell-type specific manipulations, the aBLA and pBLA in negative and positive behaviors, respectively. Interestingly, previous studies have demonstrated spatial representation of negative and positive information in the medial amygdala (62), cortical amygdala (63), and gustatory cortex (64). Thus, spatially segregated representation of negative and positive information may be a common motif throughout the central nervous system.

It is widely hypothesized that the amygdala fear circuit involves direct transmission of negative information from BLA principle neurons to CeL neurons and/or effector neurons in the CeM (65-68). Furthermore, a recent study provided evidence supporting a hypothesis that anatomical projections may be a defining structural feature of negative and positive BLA neurons. CeM-projecting BLA neurons undergo negative valence-specific synaptic changes, while NAc-projecting BLA neurons undergo positive valence-specific synaptic changes (69). Contrary to these previous models, the data obtained in studies described herein suggest that positive, but not negative BLA neurons project to the CeM and CeL. Negative, but not positive BLA neurons, project to the CeC. Moreover, both negative and positive BLA neurons project to the NAc. In regards to CeM and CeL projections, these findings are consistent with anatomical studies demonstrating that parvocellular BLA neurons (which are Ppplrlb⁺) send strong projections to the CeL and CeM and provide further support for the role of the central amygdala in appetitive behaviors (60,70-72). In regards to connections from negative BLA neurons to effector neurons in the CeM, the findings detailed herein suggest that one possible route from negative BLA neurons to the CeM is through the CeC. A recent study identified a population of Calcrl⁺ in the CeC/CeL, which supports similar negative behaviors as Rpso2⁺ BLA neurons, and, thus, may be an intermediate between negative BLA neurons and CeM effector neurons (73). In regards to NAc projections, activation of NAc-projecting BLA fibers was previously shown to support positive behaviors (45). At the same time, NAc-projecting BLA neurons reside predominantly, but not exclusively, in the posterior end of the BLA (74). However, stimulation of Rspo2⁺ BLA fibers in the NAc elicited fear-related behaviors and did not support positive behaviors, the same results obtained by soma stimulation of Rpso2⁺ BLA neurons (FIG. 27). Furthermore, a recent study demonstrated that BLA to NAc projections are necessary for place avoidance (75). Thus, NAc projections are a shared structural feature, rather than a distinct feature, of negative and positive BLA neurons.

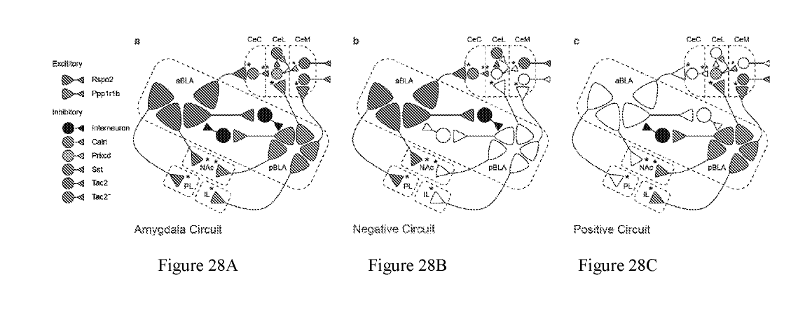

Studies on fear extinction have hypothesized on the identity of fear and fear inhibiting BLA pyramidal cells. In viva electrophysiological studies have suggested that fear and fear inhibiting neurons are spatially intermingled (76,77). However, in contrast to the intermingling of fear and fear inhibiting neurons, the results of the studies disclosed herein suggest that fear inhibiting principle neurons are amongst the Ppplrlb⁺ BLA neuron, thus, spatially segregated from fear neurons (Rspo2⁺) (though at particular points in the AP axis of the BLA Rspo2 and Ppplrlb⁺ BLA neurons could appear intermingled). Behaviorally, activation of Ppplrlb⁺ BLA supports inhibition of freezing and not negative behaviors. Anatomically, Ppplrlb⁺ BLA neurons project to the CeL and the IL, areas implicated in fear inhibition and extinction (67,76,78-80). Functionally, Ppplrlb⁺ BLA neurons inhibit Rspo2⁺ BLA neurons, which is consistent with previous observations of the inhibition of BLA fear neurons during extinction (77). Therefore, it is now proposed that fear-inhibiting BLA principle neurons that emerge during fear extinction protocols are a subset of Ppplrlb+ BLA neurons. Overall, the identification of genetic markers for distinct populations of BLA neurons has permitted the functional and anatomical dissociation of the circuit underlying negative and positive behaviors, in turn, providing a revised functional and structural model of the BLA (FIG. 28).

The experiments and results presented in Example 2 support three final conclusions about the BLA. 1) Negative and positive stimuli evoke evolutionarily determined, innate stereotypic behaviors, suggesting that negative and positive behaviors are mediated by parallel neural circuits. Genetically defined populations of BLA neurons that mediate negative and positive behaviors suggest that valence is inherent to distinct populations of neurons in the BLA. Therefore, negative and positive information upstream and downstream of the BLA must be segregated (not necessarily spatially) and also may be genetically distinct. 2) The BLA neurons that mediate negative and positive behaviors reside in two distinct subnuclei, the aBLA and pBLA. The aBLA and pBLA make predominately reciprocal inhibitory connections and project to distinct brain regions. Therefore, in regard to the larger brain circuit, these two distinct populations of neurons must be in parallel and the balance of excitation between these two populations provides a mechanism of representing a continuous range of negative and positive information. 3) Emotionally neutral stimuli can be arbitrarily associated to negative or positive behaviors. The BLA supports the association of valence to neutral stimuli, as do several other neuronal populations throughout the brain (81-83). However, because the BLA receives input from associative brain regions, such as the hippocampus and sensory cortices, BLA neurons have been implicated to be directly involved in associative functions (84-87). The data supports a conclusion that upstream neurons carrying neutral information that can be associated to negative and positive behaviors, must directly or indirectly diverge onto both populations of BLA neurons. Overall, the basolateral amygdala is a hub for the antagonistic control of valence-specific behaviors.

### REFERENCES

1. Caspi, A. et al. Influence of life stress on depression: moderation by a polymorphism in the 5-HTT gene. Science 301, 386-389 (2003).
2. Pittenger, C. & Duman, R. S. Stress, depression, and neuroplasticity: a convergence of mechanisms. Neuropsychopharmacology 33, 88-109 (2008).
3. Hyman, S. E. Revitalizing psychiatric therapeutics. Neuropsychopharmacology 39, 220-229 (2014).
4. Covington, H. E. III et al. Antidepressant effect of optogenetic stimulation of the medial prefrontal cortex. J. Neurosci. 30, 16082-16090 (2010).
5. Russo, S. J. & Nestler, E. J. The brain reward circuitry in mood disorders. Nature Rev. Neurosci. 14, 609-625 (2013).
6. Liu, X. et al. Optogenetic stimulation of a hippocampal engram activates fear memory recall. Nature 484, 381-385 (2012).
7. Ramirez, S. et al. Creating a false memory in the hippocampus. Science 341, 387-391 (2013).
8. Redondo, R. L. et al. Bidirectional switch of the valence associated with a hippocampal contextual memory engram. Nature 513, 426-430 (2014).
9. Seligman, M. E. P., Rashid, T. & Parks, A. C. Positive psychotherapy. Am. Psychol. 61,774-788 (2006).
10. Tye, K. M. et al. Dopamine neurons modulate neural encoding and expression of depression-related behaviour. Nature 493, 537-541 (2013).
11. Warden, M. R. et al. A prefrontal cortex-brain stem neuronal projection that controls response to behavioural challenge. Nature 492, 428-432 (2012).
12. Deisseroth, K. Circuit dynamics of adaptive and maladaptive behaviour. Nature 505, 309-317 (2014).
13. Lim, B. K., Huang, K. W., Grueter, B. A., Rothwell, P. E. & Malenka, R. C. Anhedonia requires MC4R-mediated synaptic adaptations in nucleus accumbens. Nature 487, 183-189 (2012).
14. Snyder, J. S., Soumier, A., Brewer, M., Pickel, J. & Cameron, H. A. Adult hippocampal neurogenesis buffers stress responses and depressive behaviour. Nature 476, 458-461 (2011).
15. Lammel, S. et al. Input-specific control of reward and aversion in the ventral tegmental area. Nature 491, 212-217 (2012).
16. Dôlen, G., Darvishzadeh, A., Huang, K. W. & Malenka, R. C. Social reward requires coordinated activity of nucleus accumbens oxytocin and serotonin. Nature 501, 179-184 (2013).
17. Schlaepfer, T. E. et al. Deep brain stimulation to reward circuitry alleviates anhedonia in refractory major depression. Neuropsychopharmacology 33, 368-377 (2008).
18. Xiu, J. et al. Visualizing an emotional valence map in the limbic forebrain by TAIFISH. Nature Neurosci. 17, 1552-1559 (2014).
19. Stuber, G. D. et al. Excitatory transmission from the amygdala to nucleus accumbens facilitates reward seeking. Nature 475, 377-380 (2011).
20. DeRubeis, R. J., Siegle, G. J. & Hollon, S. D. Cognitive therapy versis medication. Nature Rev. Neurosci. 9, 788-796 (2008).
21. Han, X. et al. A high-light sensitivity optical neural silencer: development and application to optogenetic control of non-human primate cortex. Front. Syst. Neurosci. 5, 18 (2011).
22. Brody, A. L. et al. Regional brain metabolic changes in patients with major depression treated with either paroxetine or interpersonal therapy. Arch. Gen. Psychiatry 58, 631-640 (2001).
23. Airan, R. D. et al. High-speed imaging reveals neurophysiological links to behavior in an animal model of depression. Science 317, 819-823 (2007).
24. Seki, T. & Arai, Y. Highly polysialylated neural cell adhesion molecule (NCAM-H) is expressed by newly generated granule cells in the dentate gyrus of the adult rat. J. Neurosci. 13, 2351-2358 (1993).
25. Santarelli, L. et al. Requirement of hippocampal neurogenesis for the behavioral effects of antidepressants. Science 301, 805-809 (2003).
26. Roozendaal, B., McEwen, B. S. & Chattarji, S. Stress, memory and the amygdala. Nature Rev. Neurosci. 10, 423-433 (2009).
27. Felix-Ortiz, A. C. et al. BLA to vHPC inputs modulate anxiety-related behaviors. Neuron 79, 658-664 (2013).
28. Berman, R. M. et al. Antidepressant effects of ketamine in depressed patients. Biol. Psychiatry 47, 351-354 (2000).
29. Friedman, A. K. et al. Enhancing depression mechanisms in midbrain dopamine neurons achieves homeostatic resilience. Science 344, 313-319 (2014).
30. Tsankova, N., Renthal, W., Kumar, A. & Nestler, E. J. Epigenetic regulation in psychiatric disorders. Nature Rev. Neurosci. 8, 355-367 (2007).
31. Reijmers, L. G., Perkins, B. L., Matsuo, N. & Mayford, M. Localization of a stable neural correlate of associative memory. Science 317, 1230-1233 (2007).
32. Redondo, R. L. et al. Bidirectional switch of the valence associated with a hippocampal contextual memory engram. Nature 513, 426-430 (2014).
33. Ramirez, S. et al. Creating a false memory in the hippocampus. Science 341, 387-391 (2013).
34. Liu, X. et al. Optogenetic stimulation of a hippocampal engram activates fear memory recall. Nature 484, 381-385 (2012).
35. Han, X. et al. A high-light sensitivity optical neural silencer: development and application to optogenetic control of non-human primate cortex. Front. Syst. Neurosci. 5, 18(2011).
36. Huff, M. L., Miller, R. L., Deisseroth, K., Moorman, D. E. & LaLumiere, R. T. Posttraining optogenetic manipulations of basolateral amygdala activity modulate consolidation of inhibitory avoidance memory in rats. Proc. Natl Acad. Sci. USA 110, 3597-3602 (2013).
37. Snyder, J. S., Soumier, A., Brewer, M., Pickel, J. & Cameron, H. A. Adult hippocampal neurogenesis buffers stress responses and depressive behaviour. Nature 476, 458-461 (2011).
38. Pitkanen, A., Savander, V. & LeDoux, J. E. Organization of intra-amygdaloid circuitries in the rat: an emerging framework for understanding functions of the amygdala. Trends in neurosciences 20, 517-523 (1997).
39. McDonald, A. J. Neuronal organization of the lateral and basolateral amygdaloid nuclei in the rat. The Journal of comparative neurology 222, 589-606, (1984).
40. McDonald, A. J. Neurons of the lateral and basolateral amygdaloid nuclei: a Golgi study in the rat. The Journal of comparative neurology 212, 293-312, (1982).
41. Carlsen, J. & Heimer, L. The Basolateral Amygdaloid Complex as a Cortical-Like Structure. Brain Res 441, 377-380, (1988).
42. Mascagni, F. & McDonald, A. J. A novel subpopulation of 5-HT type 3A receptor subunit immunoreactive interneurons in the rat basolateral amygdala. Neuroscience 144, 1015-1024, (2007).
43. McDonald, A. J., Muller, J. F. & Mascagni, F. GABAergic innervation of alpha type II calcium/calmodulin-dependent protein kinase immunoreactive pyramidal neurons in the rat basolateral amygdala. The Journal of comparative neurology 446, 199-218 (2002).
44. Redondo, R. L. et al. Bidirectional switch of the valence associated with a hippocampal contextual memory engram. Nature (2014).
45. Stuber, G. D. et al. Excitatory transmission from the amygdala to nucleus accumbens facilitates reward seeking. Nature 475, 377-380, (2011).
46. Ambroggi, F., Ishikawa, A., Fields, H. L. & Nicola, S. M. Basolateral amygdala neurons facilitate reward-seeking behavior by exciting nucleus accumbens neurons. Neuron 59, 648-661, (2008).
47. Goosens, K. A. & Maren, S. Contextual and auditory fear conditioning are mediated by the lateral, basal, and central amygdaloid nuclei in rats. Learning & memory 8, 148-155, (2001).
48. Cador, M., Robbins, T. W. & Everitt, B. J. Involvement of the Amygdala in Stimulus Reward Associations—Interaction with the Ventral Striatum. Neuroscience 30, 77-86, (1989).
49. Killcross, S., Robbins, T. W. & Everitt, B. J. Different types of fear-conditioned behaviour mediated by separate nuclei within amygdala. Nature 388, 377-380, (1997).
50. Gore, F. et al. Neural Representations of Unconditioned Stimuli in Basolateral Amygdala Mediate Innate and Learned Responses. Cell 162, 134-145, (2015).
51. Paton, J. J., Belova, M. A., Morrison, S. E. & Salzman, C. D. The primate amygdala represents the positive and negative value of visual stimuli during learning. Nature 439, 865-870, (2006).
52. Zirlinger, M., Kreiman, G. & Anderson, D. J. Amygdala-enriched genes identified by microarray technology are restricted to specific amygdaloid subnuclei. Proceedings of the National Academy of Sciences of the United States of America 98, 5270-5275, (2001).
53. Heiman, M. et al. A translational profiling approach for the molecular characterization of CNS cell types. Cell 135, 738-748, (2008).
54. Knight, Z. A. et al. Molecular profiling of activated neurons by phosphorylated ribosome capture. Cell 151, 1126-1137, (2012).
55. Sanz, E. et al. Cell-type-specific isolation of ribosome-associated mRNA from complex tissues. Proceedings of the National Academy of Sciences of the United States of America 106, 13939-13944, (2009).
56. Gay, L. et al. Mouse TU tagging: a chemical/genetic intersectional method for purifying cell type-specific nascent RNA. Gene & development 27, 98-115, (2013).
57. Yang, Z., Edenberg, H. J. & Davis, R. L. Isolation of mRNA from specific tissues of Drosophila by mRNA tagging. Nucleic acids research 33, e148, (2005).
58. Lein, E. S. et al. Genome-wide atlas of gene expression in the adult mouse brain. Nature 445, 168-176, (2007).
59. Hemmings, H. C., Jr., Greengard, P., Tung, H. Y. & Cohen, P. DARPP-32, a dopamine regulated neuronal phosphoprotein, is a potent inhibitor of protein phosphatase-1. Nature 310, 503-505 (1984).
60. Savander, V., Go, C. G., Ledoux, J. E. & Pitkanen, A. Intrinsic Connections of the Rat Amygdaloid Complex—Projections Originating in the Basal Nucleus. Journal of Comparative Neurology 361, 345-368, (1995).
61. Kantak, K. M., Black, Y., Valencia, E., Green-Jordan, K. & Eichenbaum, H. B. Dissociable effects of lidocaine inactivation of the rostral and caudal basolateral amygdala on the maintenance and reinstatement of cocaine-seeking behavior in rats. The Journal of neuroscience: the official journal of the Society for Neuroscience 22, 1126-1136 (2002).
62. Choi, G. B. et al. Lhx6 delineates a pathway mediating innate reproductive behaviors from the amygdala to the hypothalamus. Neuron 46, 647-660, (2005).
63. Root, C. M., Denny, C. A., Hen, R. & Axel, R. The participation of cortical amygdala in innate, odour-driven behaviour. Nature 515, 269-273, (2014).
64. Peng, Y. et al. Sweet and bitter taste in the brain of awake behaving animals. Nature 527, 512-515, (2015).
65. Herry, C. & Johansen, J. P. Encoding of fear learning and memory in distributed neuronal circuits. Nature neuroscience 17, 1644-1654, (2014).
66. Duvarci, S. & Pare, D. Amygdala microcircuits controlling learned fear. Neuron 82, 966-980, (2014).
67. Tovote, P., Fadok, J. P. & Luthi, A. Neuronal circuits for fear and anxiety. Nature reviews. Neuroscience 16, 317-331, (2015).
68. Janak, P. H. & Tye, K. M. From circuits to behaviour in the amygdala. Nature 517, 284-292, (2015).
69. Namburi, P. et al. A circuit mechanism for differentiating positive and negative associations. Nature 520, 675-678, (2015).
70. Parkinson, J. A., Robbins, T. W. & Everitt, B. J. Dissociable roles of the central and basolateral amygdala in appetitive emotional learning. The European journal of neuroscience 12, 405-413 (2000).
71. Cai, H., Haubensak, W., Anthony, T. E. & Anderson, D. J. Central amygdala PKCdelta(+) neurons mediate the influence of multiple anorexigenic signals. Nature neuroscience 17, 1240-1248, (2014).
72. Gallagher, M., Graham, P. W. & Holland, P. C. The amygdala central nucleus and appetitive Pavlovian conditioning: lesions impair one class of conditioned behavior. The Journal of neuroscience: the official journal of the Society for Neuroscience 10, 1906-1911 (1990).
73. Han, S., Soleiman, M. T., Soden, M. E., Zweifel, L. S. & Palmiter, R. D. Elucidating an Affective Pain Circuit that Creates a Threat Memory. Cell 162, 363-374, (2015).
74. McDonald, A. J. Topographical organization of amygdaloid projections to the caudatoputamen, nucleus accumbens, and related striatal-like areas of the rat brain. Neuroscience 44, 15-33, (1991).
75. Ramirez, F., Moscarello, J. M., LeDoux, J. E. & Sears, R. M. Active avoidance requires a serial basal amygdala to nucleus accumbens shell circuit. The Journal of neuroscience: the official journal of the Society for Neuroscience 35, 3470-3477, (2015).
76. Senn, V. et al. Long-range connectivity defines behavioral specificity of amygdala neurons. Neuron 81, 428-437, (2014).
77. Herry, C. et al. Switching on and off fear by distinct neuronal circuits. Nature 454, 600-606, (2008).
78. Sierra-Mercado, D., Padilla-Coreano, N. & Quirk, G. J. Dissociable roles of prelimbic and infralimbic cortices, ventral hippocampus, and basolateral amygdala in the expression and extinction of conditioned fear. Neuropsychopharmacology: official publication of the American College of Neuropsychopharmacology 36, 529-538, (2011).
79. Haubensak, W. et al. Genetic dissection of an amygdala microcircuit that gates conditioned fear. Nature 468, 270-276, (2010).
80. Kita, H. & Kitai, S. T. Amygdaloid projections to the frontal cortex and the striatum in the rat. The Journal of comparative neurology 298, 40-49, (1990).
81. Han, S., Soleiman, M. T., Soden, M. E., Zweifel, L. S. & Palmiter, R. D. Elucidating an Affective Pain Circuit that Creates a Threat Memory. Cell 162, 363-374, (2015).
82. Kunwar, P. S. et al. Ventromedial hypothalamic neurons control a defensive emotion 50 state. eLife 4, (2015).
83. Olds, J. & Milner, P. Positive reinforcement produced by electrical stimulation of septal area and other regions of rat brain. Journal of comparative and physiological psychology 47, 419-427 (1954).
84. Shi, C. J. & Cassell, M. D. Cascade projections from somatosensory cortex to the rat basolateral amygdala via the parietal insular cortex. Journal of Comparative Neurology 399, 469-491, (1998).
85. Maren, S. & Fanselow, M. S. The amygdala and fear conditioning: Has the nut been cracked? Neuron 16, 237-240, (1996).
86. Fanselow, M. S. & LeDoux, J. E. Why we think plasticity underlying Pavlovian fear conditioning occurs in the basolateral amygdala. Neuron 23, 229-232 (1999).
87. Kishi, T., Tsumori, T., Yokota, S. & Yasui, Y. Topographical projection from the hippocampal formation to the amygdala: A combined anterograde and retrograde tracing study in the rat. Journal of Comparative Neurology 496, 349-368, (2006).

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

1. Method of aiding in a treatment of a mental disorder or a condition in a subject, comprising:
(a) expressing in a first cell in a subject in need of such treatment, a stimulus-activated opsin polypeptide in an amount effective to treat a mental disorder or condition in the subject; wherein activating the first cell reactivates a positive memory in the subject;
(b) contacting the expressed stimulus-activated opsin polypeptide with a stimulus suitable to activate the stimulus-activated opsin polypeptide; and
(c) modulating the contact of the stimulus with the stimulus-activated opsin polypeptide to reactivate the positive memory engram in the subject, wherein the reactivation of the positive memory aids in the treatment of the mental disorder or the condition in the subject.

2. The method of claim 1, wherein the first cell is a hippocampal neuron of the subject, optionally is a dorsal hippocampal neuron.

3. The method of claim 1, wherein first cell is in the dentate gyrus of the hippocampus.

4. The method of claim 1, wherein the first cell projects to at least one second cell in the basal lateral amygdala (BLA) of the subject.

5. The method of claim 4, wherein the second cell is a parvocellular pyramidal neuron in the BLA of the subject.

6. The method of claim 4, wherein the second cell is a Ppplrlb⁺-expressing cell.

7. The method of claim 1, wherein the suitable stimulus comprises illumination.

8. (canceled)

9. The method of claim 1, wherein reactivation of positive memory comprises reactivation of a positive memory engram.

10. The method of claim 1, wherein the stimulation is chronic stimulation.

11. (canceled)

12. The method of claim 1, wherein the mental disorder or condition is depression or post-traumatic stress disorder (PTSD).

13. The method of claim 1, wherein the stimulus-activated opsin polypeptide comprises a light-activated opsin polypeptide.

14. The method of claim 1, further comprising: altering one or more additional treatments administered to the subject to treat or assist in treating the mental disorder or condition.

15. (canceled)

16. The method of claim 1, further comprising: exposing the subject to a positive experience sufficient to activate one or more of the first cells in the hippocampus of the subject.

17. The method of claim 16, wherein exposing the subject is at one or more of: prior to step (a) and prior to step (b).

18. The method of claim 1, wherein reactivating the positive memory induces neurogenesis in the dentate gyrus of the subject.

19-21. (canceled)

22. The method of claim 1, further comprising inhibiting a Rspo2⁺-expressing cell in the subject, at a time that is one or more of: prior to, concurrent with, or subsequent to the reactivation of the positive memory in the subject.

23-58. (canceled)

59. A method of activating a Ppplrlb⁺-expressing cell in a subject, the method comprising:
(a) expressing in one or more cells in a subject a stimulus-activated opsin polypeptide, wherein the cell in which the stimulus-activated opsin polypeptide is expressed in a Ppplrlb⁺-expressing cell or in a cell that when activated, activates a Ppplrlb⁺-expressing cell in the subject: and
(b) activating the expressed stimulus-activated opsin polypeptide; wherein the activation stimulus-activated opsin polypeptide activates the Ppplrlb⁺-expressing cell in the subject.

60. The method of claim 59, wherein the Ppplrlb⁺-expressing cell is a basal lateral amygdala (BLA) cell.

61-66. (canceled)

67. A method of inhibiting an Rspo2⁺-expressing cell in a subject, the method comprising (a) expressing in one or more cells in a subject a stimulus-activated opsin polypeptide, wherein the cell in which the stimulus-activated opsin polypeptide is expressed in an Rspo2⁺-expressing cell or in a cell that when inhibited, inhibits an Rspo2⁺-expressing cell in the subject: and
(b) activating the expressed stimulus-activated opsin polypeptide; wherein the activation stimulus-activated opsin polypeptide inhibits the Rspo2⁺-expressing cell in the subject.

68. The method of claim 67, wherein the Rspo2⁺-expressing cell is a basal lateral amygdala (BLA) cell.

69-74. (canceled)


SEQUENCE LISTING (2 sequences)

SEQ ID NO: 1 - DNA, 15 bp, Artificial Sequence
  Synthetic Oligonucleotide
cagtgtgctggaatt

SEQ ID NO: 2 - DNA, 15 bp, Artificial Sequence
  Synthetic Oligonucleotide
gatatctgcagaatt